(19) United States
(12) Patent Application Publication
(10) Pub. No.: US 2012/0183960 A1
(43) Pub. Date: Jul. 19, 2012

(54) METHOD FOR IDENTIFYING E. COLI M-17

(75) Inventors: Robert A. HOERR, Shoreview, MN (US); Eileen F. Bostwick, Dayton, MN (US); Douglas Testa, Lebanon, NJ (US)

(73) Assignee: Enterologics, Inc., Saint Paul, MN (US)

(21) Appl. No.: 13/313,742

(22) Filed: Dec. 7, 2011

Related U.S. Application Data
(60) Provisional application No. 61/420,344, filed on Dec. 7, 2010.

(51) Int. Cl. C12Q 1/68

(52) U.S. Cl. 435/6.11; 435/6.15; 435/6.12

(57) ABSTRACT

A method of identifying an M17 strain of E. coli in a human biological sample is provided. The method comprises analyzing products of an amplification reaction using DNA extracted from the human biological sample and a primer pair which amplifies an M17 specific nucleic acid sequence of an M17 nucleic acid sequence, wherein the primer pair is selected from the group consisting of SEQ ID NOs: 37 and 38; SEQ ID NO: 39 and 40; and SEQ ID NOs: 45 and 46, wherein a product of the amplification reaction is indicative of an M17 strain of E. coli. Additional primers and kits comprising same are also provided.

### RELATED APPLICATION

This application claims the benefit of priority under 35 USC 119(e) of U.S. Provisional Patent Application No. 61/420,344 filed Dec. 7, 2010, the contents of which are incorporated herein by reference in their entirety.

### FIELD AND BACKGROUND OF THE INVENTION

The present invention, in some embodiments thereof, relates to a method for identifying E. coli M-17 in a biological sample and oligonucleotides capable of same.

The intestinal microflora is important for maturation of the immune system, the development of normal intestinal morphology and for maintenance of a chronic and immunologically balanced inflammatory response. The microflora reinforces the barrier function of the intestinal mucosa by preventing attachment of pathogenic microorganisms and the entry of allergens. Some members of the microflora may contribute to the body's requirements for certain vitamins, including biotin, pantothenic acid and vitamin B₁₂. Alteration of the microbial flora of the intestine, such as may occur with antibiotic use, disease and aging, can negatively affect its beneficial role.

Probiotics are a class of microorganisms defined as live microbial organisms that beneficially affect animal and human hosts. Such beneficial effects may be due to improvement of the microbial balance of the intestinal microflora and/or improvement of the properties of the indigenous microflora. The beneficial effects of probiotics may be mediated by a direct antagonistic effect against specific groups of organisms, resulting in a decrease in numbers, by an effect on their metabolism and/or by stimulation of immunity. The mechanisms underlying the proposed actions remain vastly unknown, partly as a consequence of the complexity of the gastro-intestinal ecosystem with which these biotherapeutic agents interact. Probiotics may suppress viable counts of an undesired organism by producing antibacterial compounds, by competing for nutrients or for adhesion sites. They may alter microbial metabolism by increasing or decreasing enzyme activity. Alternatively or additionally they may stimulate the immune system by increasing antibody levels or macrophage activity.

Known probiotic strains include, for example, Bifidobacteria, Lactobacillus, Lactococcus, Saccharomyces, Streptococcus thermophilus, Enterococcus and E. coli.

It is well known that under conditions where the balance of the GI microflora is adversely affected, probiotics become of potential value in restoring the GI microflora enabling the individual host to return to normal.

Recently, it was uncovered that a single species of a non-pathogenic probiotic microorganism derived from E. coli is, alone, capable of restoring normal GI flora of human and of a variety of mammals and avians. The beneficial physiological and therapeutic activity of this species in the GI tract is described in detail in U.S. Pat. No. 6,500,423, and in WO 02/43649, which are incorporated by reference as if fully set forth herein. These references teach that the Escherichia coli strain BU-230-98 ATCC Deposit No. 202226 (DSM 12799), which is an isolate of the commercially available probiotic E. coli M-17 strain, is highly effective in preventing or treating gastro-enteric infections or disorders, maintaining or reinstating normal gastro-intestinal microflora, preventing or treating diarrhea, preventing or treating gastro-enteric infection caused by an enteric pathogen, such as a Gram negative bacterium or Gram positive bacterium, preventing or treating gastro-enteric Salmonella infection, preventing or treating infectious diarrhea, caused by, for example C. difficile, Salmonella, particularly S. Shigella, Campylobacter, E. coli, Proteus, Pseudomonas or Clostridium or diarrhea resulting from antibiotic therapy, radiotherapy or chemotherapy, and/or for normalizing the physiological activity of the gastrointestinal tract. Furthermore, U.S. Patent Application No. 20040067223 teaches that strain BU-230-98 ATCC Deposit No. 202226 (DSM 12799), while altering the microbial balance in the GI tract, is highly efficacious agent for treating IBD, such as Crohn's disease and the symptoms associated therewith and for treating other idiopathic inflammation of the small and proximal intestine.

WO2007/136553 teaches identification of M17_{SNAR} by amplification of sequences therein.

### SUMMARY OF THE INVENTION

According to an aspect of some embodiments of the present invention there is provided a method of identifying an M17 strain of E. coli in a human biological sample, the method comprising analyzing products of an amplification reaction using DNA extracted from the human biological sample and a primer pair which amplifies an M17 specific nucleic acid sequence of an M17 nucleic acid sequence, wherein said primer pair is selected from the group consisting of SEQ ID NOs: 37 and 38; SEQ ID NO: 39 and 40; and SEQ ID NOs: 45 and 46, wherein a product of the amplification reaction is indicative of an M17 strain of E. coli.

According to an aspect of some embodiments of the present invention there is provided a method of identifying an M17 strain of E. coli in a human sample, the method comprising analyzing DNA extracted from the human sample for a presence or absence of at least one M17 specific nucleic acid sequence of an M17 nucleic acid sequence being selected from the group consisting of SEQ ID NOs: 3, 30, 31, 33, 34, 35 and 36 under experimental conditions, the at least one M17 specific nucleic acid sequence being distinguishable from non M17 nucleic acid sequences in the DNA under the experimental conditions, wherein a presence of the at least one M17 specific nucleic acid sequence is indicative of M17 in the human sample.

According to an aspect of some embodiments of the present invention there is provided a method of identifying an M17 strain of E. coli in a human fecal sample, the method comprising analyzing DNA extracted from the human fecal sample for a presence or absence of at least one M17 specific nucleic acid sequence of an M17 nucleic acid sequence being selected from the group consisting of SEQ ID NOs: 1-36 under experimental conditions, the at least one M17 specific nucleic acid sequence being distinguishable from non M17 nucleic acid sequences in the DNA under the experimental conditions, wherein a presence of the at least one M17 specific nucleic acid sequence is indicative of M17 in the human fecal sample.

According to an aspect of some embodiments of the present invention there is provided a kit for identifying an M17 strain of E. coli in a human fecal sample comprising at least one oligonucleotide which hybridizes under experimental conditions to an M17 polynucleotide sequence selected from the group consisting of SEQ ID NOs: 1-36, the at least one oligonucleotide being at least 13 bases, the at least one oligonucleotide not being capable of hybridizing to a non M17 polynucleotide sequence under identical experimental conditions.

According to an aspect of some embodiments of the present invention there is provided a primer pair which amplifies an M17 specific nucleic acid sequence of an M17 nucleic acid sequence being selected from the group consisting of SEQ ID NOs: 1-36 under experimental conditions and does not amplify a non-M17 specific nucleic acid sequence under the experimental conditions, each primer of the pair being at least 13 bases.

According to some embodiments of the invention, the M17 nucleic acid sequence is selected from the group consisting of SEQ ID NOs: 3, 30 and 36.

According to some embodiments of the invention, the M17 nucleic acid sequence is selected from the group consisting of SEQ ID NOs: 34 and 35.

According to some embodiments of the invention, the method further comprises quantifying an amount of M17 in the sample.

According to some embodiments of the invention, the analyzing is effected using at least one oligonucleotide being at least 13 bases which hybridizes to the M17 specific nucleic acid sequence to provide a detectable signal under the experimental conditions and which does not hybridize to the non M17 nucleic acid sequences to provide a detectable signal under the experimental conditions.

According to some embodiments of the invention, the M17 strain of E. coli is E. coli M17p (M17 parent) Deposit No. 202226 or E. coli M17SNAR Deposit No. 7295.

According to some embodiments of the invention, the biological sample comprises a fecal sample.

According to some embodiments of the invention, the at least one oligonucleotide is fully complementary to the M17 specific polynucleotide sequence.

According to some embodiments of the invention, the analyzing is effected using two oligonucleotides, each of the two oligonucleotides being at least 13 bases.

According to some embodiments of the invention, the at least one oligonucleotide comprises two oligonucleotides, wherein a second of the two oligonucleotides hybridizes to an additional M17 polynucleotide sequence under the experimental conditions.

According to some embodiments of the invention, the determining is effected by PCR analysis.

According to some embodiments of the invention, the second of the two oligonucleotides does not hybridize to a non-M17 polynucleotide sequence under the experimental conditions.

According to some embodiments of the invention, the second of the two oligonucleotides binds to a non-M17 polynucleotide sequence under the experimental conditions.

According to some embodiments of the invention, the M17 nucleic acid sequence is selected from the group consisting of SEQ ID NOs: 3, 30, 31, 33, 34, 35 and 36.

According to some embodiments of the invention, at least one of the primers of the pair hybridizes to a polynucleotide sequence which is unique to M17.

According to some embodiments of the invention, the at least one of the primers has a nucleotide sequence as set forth in SEQ ID NO: 37-40, 45, 46 and 62-573.

According to some embodiments of the invention, a first primer of the pair is as set forth in SEQ ID NO: 37 and a second primer of the pair is as set forth in SEQ ID NO: 38.

According to some embodiments of the invention, a first primer of the pair is as set forth in SEQ ID NO: 39 and a second primer of the pair is as set forth in SEQ ID NO: 40.

According to some embodiments of the invention, a first primer of the pair is as set forth in SEQ ID NO: 45 and a second primer of the pair is as set forth in SEQ ID NO: 46.

According to some embodiments of the invention, two of the primers of the primer pair hybridize to a polynucleotide sequence which is unique to M17.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C are gel images (0.8% agarose) of extracted gDNA from M17p, M17SNAR and the 72 ECOR collection E. coli culture samples. The ECOR collection E. coli culture extracted gDNA samples (ECOR1-72) are numbered above as labeled gel lanes 1-72, respectively. Lane 74* contains M17SNAR extracted gDNA.

FIG. 2 is an electropherogram of the M17p generated, 8 kb paired end library displaying expected yield and size.

FIG. 3 is a gel image of the PCR products obtained with primer pair CP11+CP12 using the extracted gDNA from the project samples: ECOR collection E. coli culture samples (ECOR1-72), the M17 Parent Strain (M17), M17SNAR (SN) and H₂O (H) as a negative control template.

FIG. 4 is a gel image of the PCR products obtained with primer pair CP13+CP14 using the extracted gDNA from the project samples: ECOR collection E. coli culture samples (ECOR1-72), the M17 Parent Strain (M17), M17SNAR (SN) and H₂O (H) as a negative control template. The artifact observed between lanes 29-30 in FIG. 3b does not correspond to an amplified DNA fragment but was most likely due to a particulate present on the surface of the transilluminator.

FIG. 5 is a gel image of the PCR products obtained with primer pair CP19+CP20 using the extracted gDNA from the project samples: ECOR collection E. coli culture samples (ECOR1-72), the M17 Parent Strain (M17), M17SNAR (SN) and H₂O (H) as a negative control template.

FIG. 6 is a growth curve of the cell cultures as determined from the average 600 nm absorbance readings over a time course 2, 4, 6, 8, 10, 12, 14, 16, 18, 20, and 24 hours measured by Nanodrop ND-1000 Spectrophotometer.

FIG. 7 is a photograph of an agarose gel illustrating DNA extracted from spiked fecal samples. M: size marker (from top down are 23, 9.0, 6.6, 4.4, 2.3, 2.0, 1.3, 1.0, 0.8, and 0.6 Kb). All lanes were 2 μl DNA isolated from samples spiked with 50 μl of the various cell culture dilutions. Lane 1: 10¹⁰ (for extraction control, not used in PCR); Lane 2: 10⁰; Lane 3: 10-¹; Lane 4: 10-²; Lane 5: 10-³; Lane 6: 10-⁴; Lane 7: 10-⁵; Lane 8: 10-⁶; Lane 9: 10-⁷; Lane 10: 10-⁸; Lane 11: 10-⁹; Lane 12: 10-¹⁰; Lane 13: 10-¹¹; Lane 14: 10-¹²; Lane 15 10-¹³; Lane 16: Stool sample with no spike. Lane 17: PBS buffer with 10-³.

FIG. 8 is a photograph of the first duplicate of an agarose gel illustrating DNA amplified using primer set CP11/CP12.

FIG. 9 is a photograph of the first duplicate of an agarose gel illustrating DNA amplified using primer set CP13/CP14.

FIG. 10 is a photograph of the first duplicate of an agarose gel illustrating DNA amplified using primer set CP19/CP20.

FIG. 11 is a photograph of the second duplicate of an agarose gel illustrating DNA amplified using primer set CP11/CP12.

FIG. 12 is a photograph of the second duplicate of an agarose gel illustrating DNA amplified using primer set CP13/CP14.

FIG. 13 is a photograph of the second duplicate of an agarose gel illustrating DNA amplified using primer set CP19/CP20.

### DESCRIPTION OF SPECIFIC EMBODIMENTS OF THE INVENTION

The present invention, in some embodiments thereof, relates to a method for identifying E. coli M-17 in a biological sample and oligonucleotides capable of same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The probiotic activities of E. coli BU-230-98, ATCC Deposit No. 202226 (DSM 12799) (M17) render it a favorable therapeutic tool for the treatment of a myriad of gastrointestinal disorders and related disorders (e.g., immune related), indicating that antibiotic resistant strains of this bacterial species may be of regulatory importance and used in combination with antibiotic treatment.

Following administration of the probiotic, identification and quantification thereof becomes important for a variety of reasons, including regulatory and determination of dose and treatment regimen.

The present inventors sequenced the genome of the M17 parent strain of E. coli in order to identify unique sequences that could be used for its specific identification. Specifically, the present inventors used the 454 Sequencing™ process which uses a sequencing by synthesis approach to generate sequence data for M17.

Sequence data available from public sources was compared to the M17p 454 sequence data in the Cross-Match software package (using the default screening settings) and 36 fragments were identified that are present the M17p strain (see Example 1, herein below) and not found in other organisms which infect human feces. Using the Blast program, the list of sequences was narrowed down further to comprise in total 3 unique fragments and 3 partially unique sequences.

The present inventors then selected primer sequences that could be used to specifically amplify M17 sequences. The selection was based on identification of primers that could amplify a DNA segment from one of the 36 fragments of the M17p bacteria under particular experimental conditions while not being capable of amplifying sequences from other sources under the same experimental conditions.

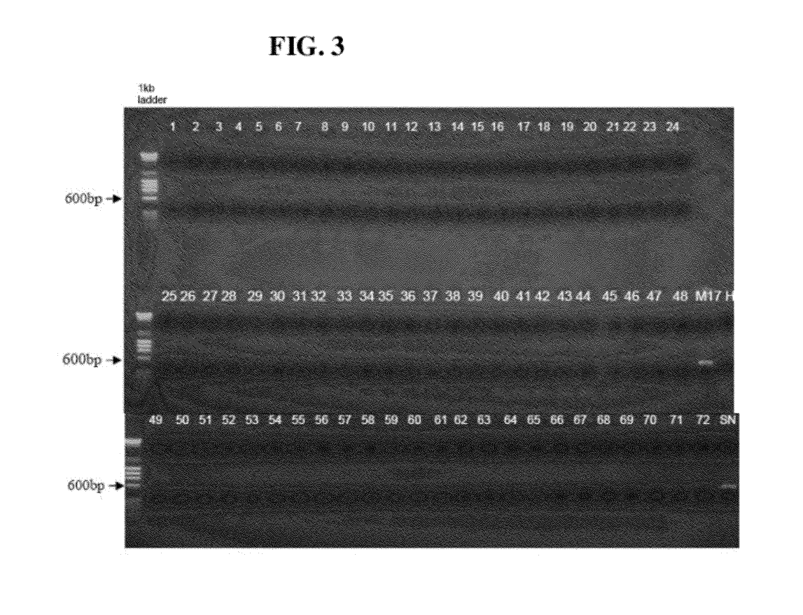

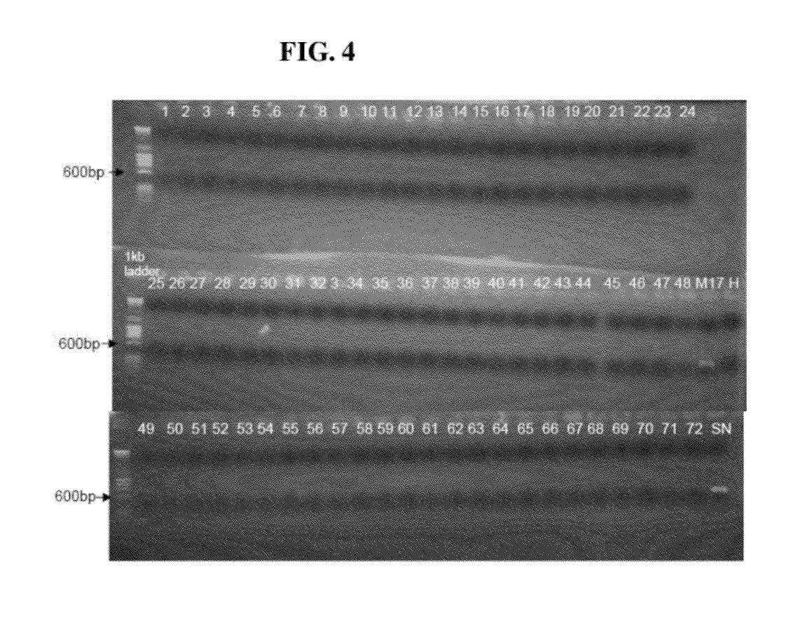

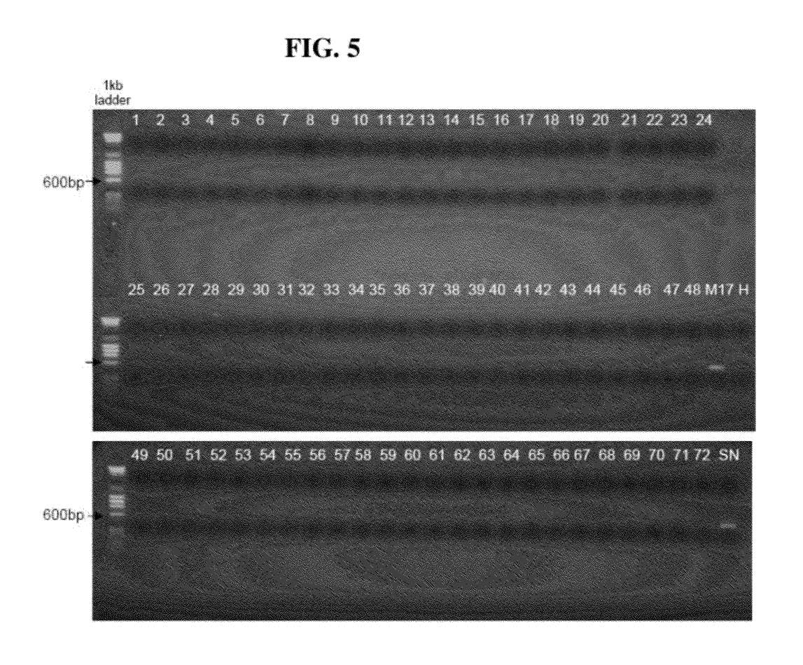

Three sets of primers were shown to be capable of specifically identifying M17 from 72 other E. coli strains (see FIGS. 3-6).

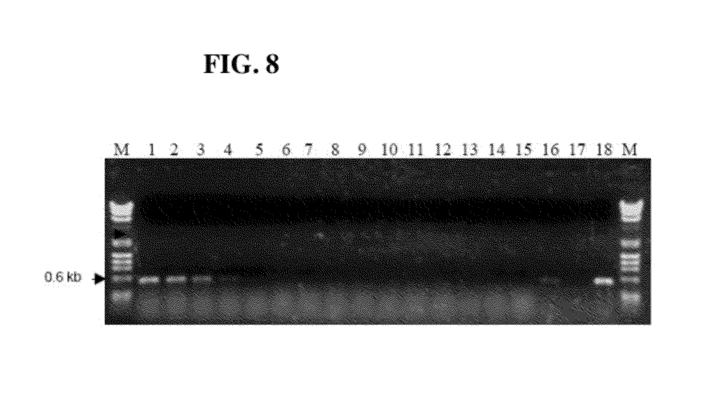

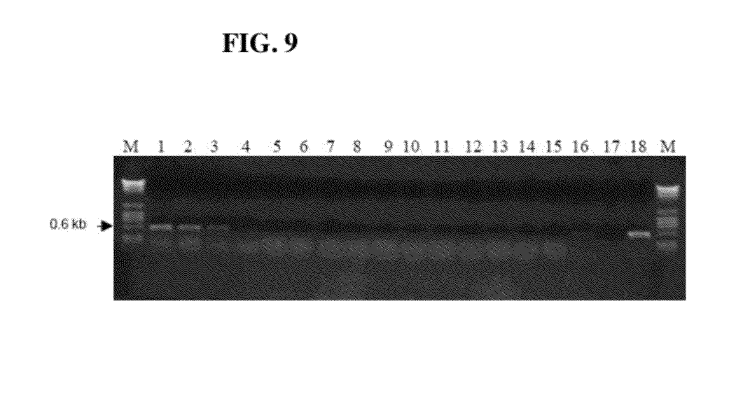

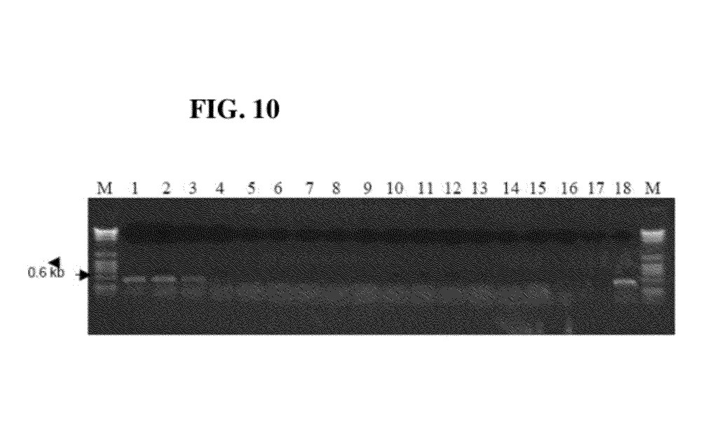

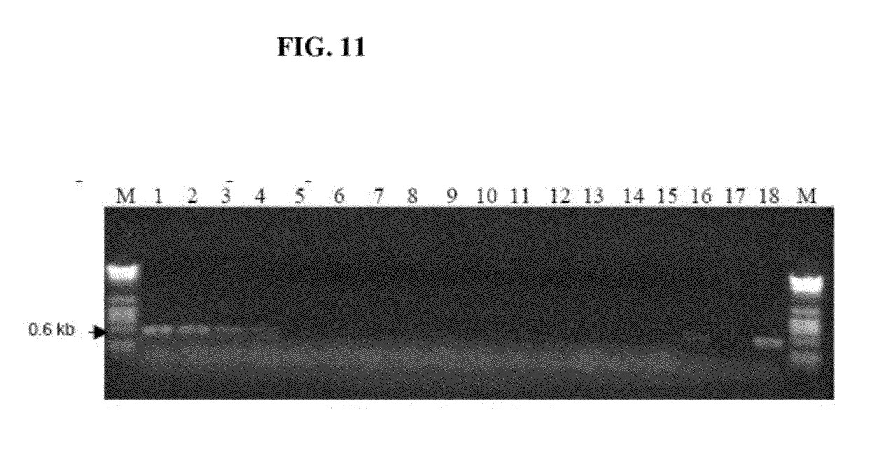

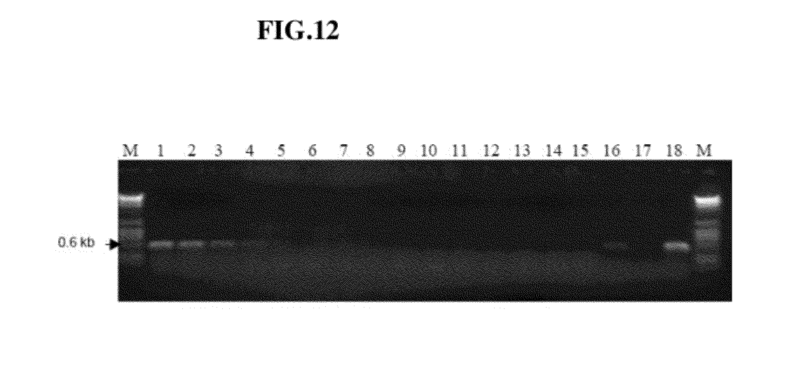

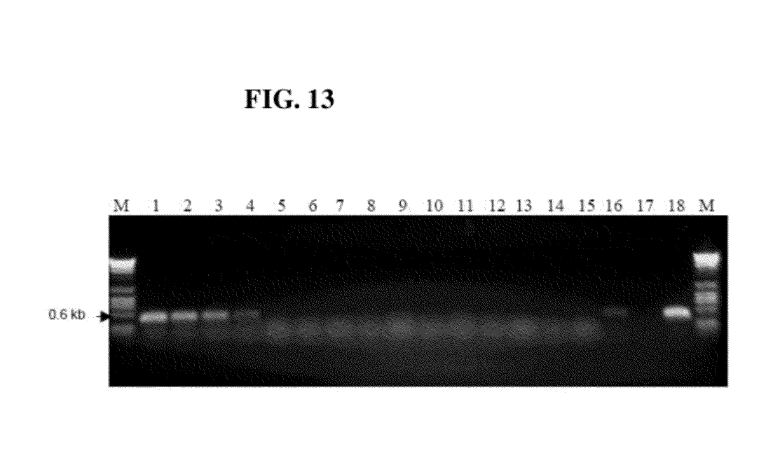

To further analyze the specificity of the three primer sets, the present inventors extracted DNA from M17p cell spiked biological stool samples. The three primer sets were able to specifically identify M17 in the stool samples (FIGS. 8-13).

The results demonstrated in the Examples section herein support the notion that the 36 fragments identified by the present inventors comprise nucleic acid sequences which may be used to distinguish M17 from other human feces infecting bacteria.

Thus, according to one aspect of the present invention, there is provided a method of identifying an M17 strain of E. coli in a human biological sample, the method comprising analyzing DNA extracted from the human biological sample for a presence or absence of at least one M17 specific nucleic acid sequence of an M17 nucleic acid sequence being selected from the group consisting of 3, 30, 31, 33, 34, 35 and 36 under experimental conditions, the at least one M17 specific nucleic acid sequence being distinguishable from non M17 nucleic acid sequences in the DNA under the experimental conditions, wherein a presence of the at least one M17 specific nucleic acid sequence is indicative of M17 in the human biological sample.

As used herein a “M17 bacterial strain of E. coli” refers to the strain per se and non-pathogenic derived strains which maintain a probiotic activity and biochemical characteristics as listed in Tables 1-3, below.

According to a particular embodiment of this aspect of the present invention, the E. coli M17 bacterial strain is BU-239, BU-230-98, BU-230-01, ATCC Deposit No. 202226 (DSM 12799). According to another embodiment, the E. coli M17 bacterial strain is a nalidixic acid-resistant mutant derivative of E. coli BU-230-98, ATCC Deposit No. 202226 (DSM 12799) such as the one deposited under the Budapest Treaty in the American Type Culture Collection (ATCC) on Dec. 22, 2005, as strain PTA-7295 (referred to herein as M17_{SNAR}).

TABLE 1
In Vitro Characterization Studies: Various E. coli Probiotic Strains

| | Strain/Code | | |
|---|---|---|---|
| | | BU 230-98 | BU 230-01 |
| | BU 239 | (BioBalance, M-17 | (BioBalance, M-17 |
| | (original M-17) | Industrial Stock) | Industrial Stock) |
| Serotype | O2 | O2 | O2 |
| Physical | Gram | Gram | Gram |
| Character- | negative | negative | negative |
| ization | rods | rods | rods |
| Metabolic | Ferments | Ferments | Ferments |
| Character- | glucose | glucose | glucose |
| ization | Reduces | Reduces | Reduces |
| | nitrates | nitrates to | nitrates to |
| | to nitrites | nitrites | nitrites |
| | Oxidase neg. | Oxidase neg. | Oxidase neg. |
| | Catalase pos. | Catalase pos. | Catalase pos. |

TABLE 2
Fermentation Profile for Various E. coli
strain M-17 Samples using API 20E

| | | E. coli | |
|---|---|---|---|
| | | strain M-17, | |
| | | ATCC 202226 | |
| | | (DSM 12799) | E. coli |
| | BU-239 | (BioBalance | strain M-17 |
| | (Original | Deposited | (Taresevich |
| Fermentation | E. coli | Master | Institute, Moscow, |
| Substrate | strain M-17) | Seed Stock) | Official Sample) |
| Ortho-nitrophenyl- | + | + | + |
| beta-D-galacto- | | | |
| pyranoside | | | |
| Arginine | − | − | − |
| dihydrolase | | | |
| Lysine | + | + | + |
| decarboxylase | | | |
| Ornithine | + | + | + |
| decarboxylase | | | |
| Citrate | − | − | − |
| H₂S | − | − | − |
| Urease | − | − | − |
| Tryptophan | − | − | − |
| deaminase | | | |
| Indole | + | + | + |
| Voges-Proskauer | − | − | − |
| Gelatin | − | − | − |
| Glucose | + | + | + |
| Mannitol | + | + | + |
| Inositiol | − | − | − |
| Sorbitol | + | + | + |
| Rhamnose | + | + | + |
| Sucrose | + | + | + |
| Melibiose | + | + | + |
| Amygdalin | − | − | − |
| Arabinose | + | + | + |

TABLE 3
In Vitro Characterization Studies: Presence of Virulence
Factors in E. coli Strain M-17 as Detected by PCR

| Category of | Type of | Virulence | E. coli Strain M-17 Isolate | | |
|---|---|---|---|---|---|
| Pathogenic | Virulence | Factor | BU-239 | ATCC 202226 | Tarasevich |
| E. coli | Factor(s) | Designation(s) | (original) | (DSM 12799) | (Russian) |
| Uropathogenic | Adhesion | Type I (Fim A) | + | + | + |
| | factors | AFA | − | − | − |
| | | SFA | − | − | − |
| Uropathogenic - | Adhesion | PapC | − | − | − |
| septicemic | factors | PapG | − | − | − |
| | (P fimbriae) | | | | |
| Uropathogenic - | Aerobactin | iuc | − | − | − |
| septicemic - | | | | | |
| meningitis assoc. | | | | | |
| Enterohemorragic - | Hemolysins | HlyA, HlyC | − | − | − |
| uropathogenic | | Ehx | − | − | − |
| Enterohemorragic - | Attaching and | pas | − | − | − |
| enteropathogenic | effacing gene | | | | |
| | Intimin | eae | − | − | − |
| Enterohemorragic | Shigatoxins | Stx1, Stx2 | − | − | − |
| | | VT2vpl, VT2vh | − | − | − |
| | | SLT I, SLT II | | | |
| | Flagellar | FliC | − | − | − |
| | antigen | | | | |
| | O serogroup | O157 | − | − | − |
| | H serotype | H7 | − | − | − |
| Enteropathogenic | Attaching and | EAE | − | − | − |
| | effacing factor | | | | |
| | Bundle | bfp | − | − | − |
| | forming pili | | | | |
| Enteroaggregative | Adhesion | aggR | − | − | − |
| | factors | AAF/1 | − | − | − |
| | Toxin | EAST1 | − | − | − |
| Enterotoxigenic | Adhesion | CFA1, | − | − | − |
| | factors | CFA2 (CS1coo) | − | − | − |
| | | CFA2 (CS3 cst) | − | − | − |
| | Adhesion | F4 (K88) | − | − | − |
| | factors (shared | F5 (K99) | − | − | − |
| | by porcine and | F18 | − | − | − |
| | bovine | F41 | − | − | − |
| | Enterotoxins | LT, StaH | − | − | − |
| | | STaP, STb | − | − | − |
| Extraintestinal | Adhesion factor | CS31a | − | − | − |
| | Autotransporter | Tsh | − | − | − |

The present invention contemplates identifying M17 in any biological sample which comprises nucleic acids (DNA and/or RNA). The biological sample typically comprises a body fluid or part of an organism. The sample may be blood, feces, semen, skin, cheek cell, urine cerebrospinal fluid and saliva. According to one embodiment, the biological sample is retrieved from a human subject. The sample may also be a food or feed sample. The sample may be fresh or frozen.

Isolation, extraction or derivation of DNA may be carried out by any suitable method. Isolating DNA from a biological sample generally includes treating a biological sample in such a manner that genomic DNA present in the sample is extracted and made available for analysis. Any isolation method that results in extracted genomic DNA may be used in the practice of the present invention. It will be understood that the particular method used to extract DNA will depend on the nature of the source.

Methods of DNA extraction are well-known in the art. A classical DNA isolation protocol is based on extraction using organic solvents such as a mixture of phenol and chloroform, followed by precipitation with ethanol (J. Sambrook et al., “Molecular Cloning: A Laboratory Manual”, 1989, 2.sup.nd Ed., Cold Spring Harbour Laboratory Press: New York, N.Y.). Other methods include: salting out DNA extraction (P. Sunnucks et al., Genetics, 1996, 144: 747-756; S. M. Aljanabi and I. Martinez, Nucl. Acids Res. 1997, 25: 4692-4693), trimethylammonium bromide salts DNA extraction (S. Gustincich et al., BioTechniques, 1991, 11: 298-302) and guanidinium thiocyanate DNA extraction (J. B. W. Hammond et al., Biochemistry, 1996, 240: 298-300).

There are also numerous versatile kits that can be used to extract DNA from tissues and bodily fluids and that are commercially available from, for example, BD Biosciences Clontech (Palo Alto, Calif.), Epicentre Technologies (Madison, Wis.), Gentra Systems, Inc. (Minneapolis, Minn.), MicroProbe Corp. (Bothell, Wash.), Organon Teknika (Durham, N.C.), and Qiagen Inc. (Valencia, Calif.). User Guides that describe in great detail the protocol to be followed are usually included in all these kits. Sensitivity, processing time and cost may be different from one kit to another. One of ordinary skill in the art can easily select the kit(s) most appropriate for a particular situation.

The sample may be processed before the method is carried out, for example DNA purification may be carried out following the extraction procedure. The DNA in the sample may be cleaved either physically or chemically (e.g. using a suitable enzyme). Processing of the sample may involve one or more of: filtration, distillation, centrifugation, extraction, concentration, dilution, purification, inactivation of interfering components, addition of reagents, and the like.

As mentioned, the method is effected by identifying at least one M17 specific nucleic acid sequence of an M17 nucleic acid sequence being selected from the group consisting of SEQ ID NOs: 3, 30, 31, 33, 34, 35 and 36.

The phrase “M17 specific nucleic acid sequence” as used herein refers to a sequence which is unique to M17 bacteria and is not present in non-M17 nucleic acid sequences. Such a sequence is detectable and distinguishable using molecular biology tools, as further described herein below.

Preferably the sequence is 100% unique (as verified using a sequence alignment software such as BLAST analysis) but it may comprise a certain level of homology/identity. Thus according to a specific embodiment, the sequence is at least no more than 70% homologous, 75% homologous, 80% homologous, 85% homologous, 90% homologous with non-M17 nucleic acid sequences.

The M17 specific nucleic is at least about 13, 16, 18, 20, 22, 25, 30, 40, 50, 55, 60, 65, 70, 80, 90, 100, 120 or more nucleotides.

According to a particular embodiment, the method is effected by identifying at least one M17 specific nucleic acid sequence of an M17 nucleic acid sequence being selected from the group consisting of SEQ ID NOs: 3, 30, 36, 47-51 and 646-647.

Sequences 47-51 and 646-647 are comprised in the SEQ ID NOs: 31, 33, 34 and 35 and have been shown by BLAST analysis not to comprise nucleic acid sequences which have more than 75% identity with another nucleic acid sequence, as detailed in Table 4 herein below.

TABLE 4

| | | alignment with | | |
|---|---|---|---|---|
| Frag- | SEQ | other strains or | Unique | Unique |
| ment | ID NO: | species | region | region |
| frag-03 | 3 | unique | all | |
| frag-30 | 30 | unique | all | |
| frag-31 | 31 | partial | 620-763 | |
| | | | SEQ ID NO: 47 | |
| frag-33 | 33 | partial | 1-322 | 1488-1750 |
| | | | SEQ ID NO: 48 | SEQ ID NO: 49 |
| frag-34 | 34 | partial | 1-188 | 1215-1407 |
| | | | SEQ ID NO: 50 | SEQ ID NO: 51 |
| frag-35 | 35 | partial | 1-393 | 492-1449 |
| | | | SEQ ID NO: 646 | SEQ ID NO: 647 |
| frag-36 | 36 | unique | all | |

*partially unique sequences are defined as a fragment having regions portions similar to a DNA sequence in the database with at least 75% identities and longer 100 nt long and portions which do not align to any sequence in the data base under these terms.
**the unique sequences are defined as a fragment lacking any region similar to a DNA sequence in the database with at least 75% identities and longer 100 nt long.

Table 5 herein below provides the alignments for SEQ ID NO: 31.

TABLE 5

| partial | | score |
|---|---|---|
| alignment | with | (local) |
| 763-881 | >gb\|CP001127.1\| | Identities = |
| | Salmonella enterica | 93/123 (75%) |
| | subsp. enterica serovar | |
| | Sc . . . 71.6 8e−09 | |
| 763-881 | >gb\|CP001846.1\| | Identities = |
| | Escherichia coli | 91/122 (74%) |
| | O55:H7 str. | |
| | CB9615, complete genome | |
| 763-881 | >gb\|CP001063.1\| | Identities = |
| | Shigella boydii | 91/122 (74%) |
| | CDC 3083-94, complete genome | |
| 101-619 | Marinobacter sp. | Identities = |
| | ELB17 1101232001211 | 393/519 (76%) |

Table 6 herein below provides the alignments for SEQ ID NO: 33.

TABLE 6

| partial | | score |
|---|---|---|
| alignment | with | (local) |
| 1750-2223 | >emb\|FP929037.1\| | Identities = |
| | Clostridium saccharolyticum- | 341/482 (70%) |
| | like K10 draft genome | |
| 1752-2329 | >emb\|AM990992.1\| | Identities = |
| | Staphylococcus aureus | 394/583 (67%) |
| | subsp. aureus ST398 | |
| | complete genome, isolate | |
| 325-900 | >emb\|AM990992.1\| | Identities = |
| | Staphylococcus aureus | 382/593 (64%) |
| | subsp. aureus ST398 | |
| | complete genome, isolate | |
| 1751-2070 | >gb\|CP000721.1\| | Identities = |
| | Clostridium beijerinckii | 228/320 (71%) |
| | NCIMB 8052, complete genome | |
| 725-917 | >gb\|CP000721.1\| | Identities = |
| | Clostridium beijerinckii | 137/196 (69%) |
| | NCIMB 8052, complete genome | |
| 582-1488 | >gb\|CP001740.1\| | Identities = |
| | Sebaldella termitidis | 601/931 (64%) |
| | ATCC 33386 plasmid pSTERM01, | |
| | complete sequence | |
| 1761-2094 | >gb\|CP001740.1\| | Identities = |
| | Sebaldella termitidis | 236/340 (69%) |
| | ATCC 33386 plasmid pSTERM01, | |
| | complete sequence | |
| 402-918 | >gb\|CP000569.1\| | Identities = |
| | Actinobacillus pleuropneumoniae | 347/529 (65%) |
| | L20 Serotype 5b complete genome | |

Table 7A herein below provides the alignments for SEQ ID NO: 34.

TABLE 7A

| partial | | score |
|---|---|---|
| alignment | with | (local) |
| 771-1214 | >gb\|CP000891.1\| | Identities = |
| | Shewanella baltica | 321/445 (72%) |
| | OS195, complete genome | |
| 188-688 | Vibrio alginolyticus | Identities |
| | | 414/559 (75%) |

Table 7B herein below provides the alignments for SEQ ID NO: 35.

TABLE 7B

| partial | | score |
|---|---|---|
| alignment | with | (local) |
| 393-491 | Populus trichocarpa | Identities = |
| | Ptrichocarpa_Cont20220 | 75/101 (75%) |

Typically, the method of this aspect of the present invention is carried out using an isolated oligonucleotide which hybridizes to an M17 nucleic acid sequence by complementary base-pairing in a sequence specific manner, and discriminates the M17 nucleic acid sequence from other nucleic acid sequences in the DNA sample. Oligonucleotides typically comprises a region of complementary nucleotide sequence that hybridizes under stringent conditions to at least about 8, 10, 13, 16, 18, 20, 22, 25, 30, 40, 50, 55, 60, 65, 70, 80, 90, 100, 120 (or any other number in-between) or more consecutive nucleotides in a target nucleic acid molecule. Depending on the particular assay, the consecutive nucleotides can either include the M17 specific nucleic acid sequence, or be a specific region in close enough proximity 5′ and/or 3′ to the M17 specific nucleic acid sequence to carry out the desired assay.

The term “isolated”, as used herein in reference to an oligonucleotide, means an oligonucleotide, which by virtue of its origin or manipulation, is separated from at least some of the components with which it is naturally associated or with which it is associated when initially obtained. By “isolated”, it is alternatively or additionally meant that the oligonucleotide of interest is produced or synthesized by the hand of man.

As mentioned herein above, the present inventors have identified 36 fragments (SEQ ID NOs:1-36) which may be used to distinguish M17 from other human feces infecting bacteria. Oligonucleotides which specifically hybridize to any one of these fragments may be used to identify M17 in human fecal samples.

In order to identify an oligonucleotide specific for any of the M17 sequences SEQ ID NOs: 1-36, the gene/transcript and/or context sequence surrounding the SNP of interest is typically examined using a computer algorithm which starts at the 5′ or at the 3′ end of the nucleotide sequence. Typical algorithms will then identify oligonucleotides of defined length that are unique to the gene/SNP context sequence, have a GC content within a range suitable for hybridization, lack predicted secondary structure that may interfere with hybridization, and/or possess other desired characteristics or that lack other undesired characteristics.

Following identification of the oligonucleotide it may be tested for specificity towards M17 under wet or dry conditions. Thus, for example, in the case where the oligonucleotide is a primer, the primer may be tested for its ability to amplify a sequence of M17 using PCR to generate a detectable product and for its non ability to amplify other bacterial strains. The products of the PCR reaction may be analyzed on a gel and verified according to presence and/or size.

Additionally, or alternatively, the sequence of the oligonucleotide may be analyzed by computer analysis to see if it is homologous (or is capable of hybridizing to) other known sequences. A BLAST 2.2.10 (Basic Local Alignment Search Tool) analysis may be performed on the chosen oligonucleotide (worldwidewebdotncbidotnlmdotnihdotgov/blast/). The BLAST program finds regions of local similarity between sequences. It compares nucleotide or protein sequences to sequence databases and calculates the statistical significance of matches thereby providing valuable information about the possible identity and integrity of the ‘query’ sequences.

According to one embodiment, the oligonucleotide is a probe. As used herein, the term “probe” refers to an oligonucleotide which hybridizes to the M17 specific nucleic acid sequence to provide a detectable signal under experimental conditions and which does not hybridize to non M17 nucleic acid sequences to provide a detectable signal under identical experimental conditions.

Below is a list of exemplary probes that may be used to identify M17 specific sequences (SEQ ID NOs: 1-36).

TABLE 8

| | | | Tm | | |
|---|---|---|---|---|---|
| | | | (50 mM | | SEQ |
| Seq ID | start | length | salt)_{—} | Sequence | ID NO: |
| frag-01 | 973 | 25 | 59.99 | TTGTCCATCTT | 574 |
| | | | | CCTGATATTGGTAT | |
| frag-01 | 730 | 20 | 60.01 | TCTTCCCGGAA | 575 |
| | | | | AATGAGATG | |
| frag-02 | 53 | 25 | 60.22 | TGTATCAAGCTT | 576 |
| | | | | TCAACGTTACTGA | |
| frag-02 | 122 | 20 | 59.98 | GTGCGGTGAAAA | 577 |
| | | | | AGGTCATT | |
| frag-03 | 1218 | 25 | 59.99 | GGTTACTTTTTGT | 578 |
| | | | | TCAAGTCAGCAT | |
| frag-03 | 1157 | 20 | 60 | GAGGGCGATAA | 579 |
| | | | | TGAAATCGA | |
| frag-04 | 5 | 25 | 59.41 | GTAGGTAAAGG | 580 |
| | | | | TCTGGATGGTAGTG | |
| frag-04 | 109 | 20 | 60.01 | TGTGTGGAATG | 581 |
| | | | | GTGCTGTTT | |
| frag-05 | 30 | 25 | 60 | CAGATACACCG | 582 |
| | | | | GATATTTAGGAATG | |
| frag-05 | 171 | 20 | 59.95 | CGTCGGGTCAA | 583 |
| | | | | GGATAGGTA | |
| frag-06 | 97 | 25 | 59.97 | CCGCTAGATAA | 584 |
| | | | | AAACTGTATTGCAT | |
| frag-06 | 264 | 20 | 60.03 | GTTGTGGAGCA | 585 |
| | | | | GCTTGAACA | |
| frag-07 | 283 | 25 | 59.96 | AAAGTGTTTTCA | 586 |
| | | | | ATTCAACAGGAAG | |
| frag-07 | 428 | 20 | 60.01 | GGTGCTAGACTC | 587 |
| | | | | TGGGCTTG | |
| frag-08 | 218 | 25 | 60.19 | GTAGTCGTCAAG | 588 |
| | | | | CCTTCATTCTTTA | |
| frag-08 | 298 | 20 | 60 | ACTAAGCAGAAG | 589 |
| | | | | CCGCCATA | |
| frag-09 | 284 | 25 | 60.17 | GTTCCGTTCCTC | 590 |
| | | | | TGGTAAATTAGTT | |
| frag-09 | 148 | 20 | 59.99 | GAGCTTTGGCTT | 591 |
| | | | | AAGGGCTT | |
| frag-10 | 202 | 25 | 60.01 | GCTTAAGTACGG | 592 |
| | | | | TGACATTGTTCTT | |
| frag-10 | 228 | 20 | 60.05 | CTACCCGTGGCA | 593 |
| | | | | CAGTAGGT | |
| frag-11 | 195 | 25 | 60.01 | GGAGAACAAAG | 594 |
| | | | | ATTTTTACCCAATT | |
| frag-11 | 9 | 20 | 59.95 | GGAAACAAACC | 595 |
| | | | | GACTGGAAA | |
| frag-12 | 415 | 25 | 60.03 | AATATATTACTG | 596 |
| | | | | GGGCTAAAGTCCG | |
| frag-12 | 445 | 20 | 59.98 | TTTCCAGTGGC | 597 |
| | | | | GATCTAGCT | |
| frag-13 | 96 | 25 | 60.04 | ATCTAATCATGT | 598 |
| | | | | ACCGACATCAGGT | |
| frag-13 | 188 | 20 | 60.1 | GGCAAGCAGA | 599 |
| | | | | TTGTATCGGT | |
| frag-14 | 44 | 25 | 59.99 | CTTGGTATTGGG | 600 |
| | | | | AAAAAGATATCCT | |
| frag-14 | 124 | 20 | 60.04 | GAAATTATGG | 601 |
| | | | | GAGCAAGGCA | |
| frag-15 | 203 | 25 | 60.03 | GACGGATAAAC | 602 |
| | | | | AGATCCACAATTAC | |
| frag-15 | 132 | 20 | 59.97 | GGGCAGACTA | 603 |
| | | | | TCAGGCAGAG | |
| frag-16 | 104 | 25 | 60.38 | GTCAGACAGGC | 604 |
| | | | | AAATCCATAGATAG | |
| frag-16 | 24 | 20 | 59.96 | GAGGCATAAA | 605 |
| | | | | CCCATGCTGT | |
| frag-17 | 130 | 25 | 60.02 | GGACATTAATA | 606 |
| | | | | TCTGTGGGTGAGTC | |
| frag-17 | 56 | 20 | 59.91 | TTGAATTTATT | 607 |
| | | | | CGCCCGAAC | |
| frag-18 | 194 | 25 | 59.98 | GAGAATGTGACG | 608 |
| | | | | TTTATGTGTTCAG | |
| frag-18 | 444 | 20 | 60.01 | CCAGTCAGTGA | 609 |
| | | | | GCTATGGCA | |
| frag-19 | 85 | 25 | 59.97 | AGAGCGTTAAG | 610 |
| | | | | TTTTGGTATCAATG | |
| frag-19 | 111 | 20 | 60.06 | ACAGCAACTG | 611 |
| | | | | CGTCTTTCCT | |
| frag-20 | 44 | 25 | 60.04 | TCTTTCACTGC | 612 |
| | | | | ATAAATTAAATGCA | |
| frag-20 | 229 | 20 | 60.07 | TCAACCTAATG | 613 |
| | | | | CAAATGCCA | |
| frag-21 | 720 | 25 | 60 | TTCTCTTGAGCG | 614 |
| | | | | AAGTGTTTTAGTT | |
| frag-21 | 146 | 20 | 59.98 | ACGCCAGAGAA | 615 |
| | | | | TCTGGCTAA | |
| frag-22 | 130 | 25 | 60.05 | TTGTTATCACTGA | 616 |
| | | | | ATACTTGGGGTT | |
| frag-22 | 501 | 20 | 60.05 | CCCGTTTGGG | 617 |
| | | | | TGATAATGTC | |
| frag-23 | 152 | 25 | 60.02 | TATCACTGTTAG | 618 |
| | | | | GTTGGGAATGAAT | |
| frag-23 | 87 | 20 | 60 | GAAAAGGTTGC | 619 |
| | | | | TTGACGCTC | |
| frag-24 | 587 | 25 | 59.99 | GAGATAATGAG | 620 |
| | | | | TCCTCTTCTTTCCC | |
| frag-24 | 31 | 20 | 60.03 | GGGTTGGATCA | 621 |
| | | | | TTGTTCCAC | |
| frag-25 | 214 | 25 | 60 | CATTAGGACTTTT | 622 |
| | | | | GTGCACCTTAGT | |
| frag-25 | 237 | 20 | 59.98 | GTCGCTTTGCTG | 623 |
| | | | | CATATTGA | |
| frag-26 | 877 | 25 | 60 | CAGTAATCGTTT | 624 |
| | | | | TACTGTCCGAACT | |
| frag-26 | 800 | 20 | 60.02 | TCTCGATGTACT | 625 |
| | | | | GCTGGTGC | |
| frag-27 | 105 | 25 | 60.02 | CAGCTTCGACTT | 626 |
| | | | | GTATCAGTAGACA | |
| frag-27 | 58 | 20 | 60 | ATACGTTTTCA | 627 |
| | | | | CGCCGTTTC | |
| frag-28 | 543 | 25 | 59.99 | AACGACGTAAAG | 628 |
| | | | | AACTCAAAATGAC | |
| frag-28 | 682 | 20 | 59.99 | CGACCCTAATTG | 629 |
| | | | | GCTGTTGT | |
| frag-29 | 149 | 25 | 59.97 | AGTTATCGACTA | 630 |
| | | | | TCAACGGTGAAAG | |
| frag-29 | 116 | 20 | 60.02 | GCGGTGGCTA | 631 |
| | | | | CACTATGGTT | |
| frag-30 | 532 | 25 | 59.98 | CACCTGAACTTC | 632 |
| | | | | TTGAGAGAGTTTC | |
| frag-30 | 817 | 20 | 60 | ATCGCGGTAAC | 633 |
| | | | | ACTTGGTTC | |
| frag-31 | 427 | 25 | 60 | AGCAAGTCTCTCA | 634 |
| | | | | AAACCTACAGAA | |
| frag-31 | 502 | 20 | 59.96 | TTTACCTATGG | 635 |
| | | | | CTGTTGCCC | |
| frag-32 | 28 | 25 | 59.81 | TTTTTGTTAAATG | 636 |
| | | | | ATGCGCATTATA | |
| frag-32 | 28 | 20 | 57.87 | TTTTTGTTAAA | 637 |
| | | | | TGATGCGCA | |
| frag-33 | 2355 | 25 | 59.99 | AAAAATAGATG | 638 |
| | | | | ATAACGGAAAAGGG | |
| frag-33 | 750 | 20 | 60.01 | TGGTGATATTTC | 639 |
| | | | | GTCCCCAT | |
| frag-34 | 1067 | 25 | 60.02 | GAAAATGGTAAGA | 640 |
| | | | | AAGAAGCATTGA | |
| frag-34 | 37 | 20 | 60.02 | CGCTGTGGAAA | 641 |
| | | | | GTGACAGAA | |
| frag-35 | 939 | 25 | 59.99 | TACCGCTGTATTA | 642 |
| | | | | AATTAGTGTGCA | |
| frag-35 | 340 | 20 | 59.98 | TGCGAATGAAC | 643 |
| | | | | TCACAGGAG | |
| frag-36 | 392 | 25 | 60.02 | GGATACGAGCAA | 644 |
| | | | | ATAATACATCACC | |
| frag-36 | 235 | 20 | 60.02 | ACAGTCGAGCC | 645 |
| | | | | AGCTTCAAT | |

The probes of this embodiment of this aspect of the present invention may be, for example, affixed to a solid support (e.g., arrays or beads).

According to another embodiment, the oligonucleotide is a primer of a primer pair. As used herein, the term “primer” refers to an oligonucleotide which acts as a point of initiation of a template-directed synthesis using methods such as PCR (polymerase chain reaction) or LCR (ligase chain reaction) under appropriate conditions (e.g., in the presence of four different nucleotide triphosphates and a polymerization agent, such as DNA polymerase, RNA polymerase or reverse-transcriptase, DNA ligase, etc, in an appropriate buffer solution containing any necessary co-factors and at suitable temperature(s)). Such a template directed synthesis is also called “primer extension”. For example, a primer pair may be designed to amplify a region of DNA using PCR. Such a pair will include a “forward primer” and a “reverse primer” that hybridize to complementary strands of a DNA molecule and that delimit a region to be synthesized/amplified. A primer of this aspect of the present invention is capable of amplifying, together with its pair (e.g. by PCR) an M17 specific nucleic acid sequence to provide a detectable signal under experimental conditions and which does not amplify non M17 nucleic acid sequence to provide a detectable signal under identical experimental conditions.

According to additional embodiments, the oligonucleotide is about 8, 9, 10, 11, 12, 13, 14, 15, 16, 17, 18, 19, 20, 21, 22, 23, 24 or 25 nucleotides in length. While the maximal length of a probe can be as long as the target sequence to be detected, depending on the type of assay in which it is employed, it is typically less than about 50, 60, 65, or 70 nucleotides in length. In the case of a primer, it is typically less than about 30 nucleotides in length. In a specific preferred embodiment of the invention, a primer or a probe is within the length of about 18 and about 28 nucleotides. It will be appreciated that when attached to a solid support, the probe may be of about 30-70, 75, 80, 90, 100, or more nucleotides in length.

The oligonucleotide of this aspect of the present invention need not reflect the exact sequence of the M17 specific nucleic acid sequence (i.e. need not be fully complementary), but must be sufficiently complementary to hybridize with the M17 specific nucleic acid sequence under the particular experimental conditions. Accordingly, the sequence of the oligonucleotide typically has at least 70% homology, preferably at least 80%, 90%, 95%, 97%, 99% or 100% homology, for example over a region of at least 13 or more contiguous nucleotides with the target M17 nucleic acid sequence. The conditions are selected such that hybridization of the oligonucleotide to the M17 nucleic acid sequence is favored and hybridization to other non M17 nucleic acid sequences is minimized.

By way of example, hybridization of short nucleic acids (below 200 bp in length, e.g. 13-50 bp in length) can be effected by the following hybridization protocols depending on the desired stringency; (i) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 1-1.5° C. below the Tm, final wash solution of 3 M TMACI, 0.01 M sodium phosphate (pH 6.8), 1 mM EDTA (pH 7.6), 0.5% SDS at 1-1.5° C. below the Tm (stringent hybridization conditions) (ii) hybridization solution of 6×SSC and 0.1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tm, final wash solution of 3 M TMACI, 0.01 M sodium phosphate (pH 6.8), 1 mM EDTA (pH 7.6), 0.5% SDS at 1-1.5° C. below the Tm, final wash solution of 6×SSC, and final wash at 22° C. (stringent to moderate hybridization conditions); and (iii) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature at 2.5-3° C. below the Tm and final wash solution of 6×SSC at 22° C. (moderate hybridization solution).

Various considerations must be taken into account when selecting the stringency of the hybridization conditions. For example, the more closely the oligonucleotide reflects a sequence that is present in the non-M17 nucleic acid, the higher the stringency of the assay conditions should be, although the stringency must not be too high so as to prevent hybridization of the oligonucleotides to the M17 specific nucleic acid sequence. Further, the lower the homology of the oligonucleotide to the M17 specific nucleic acid sequence, the lower the stringency of the assay conditions should be, although the stringency must not be too low to allow hybridization to non M17 specific nucleic acid sequences.

Oligonucleotides of the invention may be prepared by any of a variety of methods (see, for example, J. Sambrook et al., “Molecular Cloning: A Laboratory Manual”, 1989, 2.sup.nd Ed., Cold Spring Harbour Laboratory Press: New York, N.Y.; “PCR Protocols: A Guide to Methods and Applications”, 1990, M. A. Innis (Ed.), Academic Press: New York, N.Y.; P. Tijssen “Hybridization with Nucleic Acid Probes—Laboratory Techniques in Biochemistry and Molecular Biology (Parts I and II)”, 1993, Elsevier Science; “PCR Strategies”, 1995, M. A. Innis (Ed.), Academic Press: New York, N.Y.; and “Short Protocols in Molecular Biology”, 2002, F. M. Ausubel (Ed.), 5.sup.th Ed., John Wiley & Sons: Secaucus, N.J.). For example, oligonucleotides may be prepared using any of a variety of chemical techniques well-known in the art, including, for example, chemical synthesis and polymerization based on a template as described, for example, in S. A. Narang et al., Meth. Enzymol. 1979, 68: 90-98; E. L. Brown et al., Meth. Enzymol. 1979, 68: 109-151; E. S. Belousov et al., Nucleic Acids Res. 1997, 25: 3440-3444; D. Guschin et al., Anal. Biochem. 1997, 250: 203-211; M. J. Blommers et al., Biochemistry, 1994, 33: 7886-7896; and K. Frenkel et al., Free Radic. Biol. Med. 1995, 19: 373-380; and U.S. Pat. No. 4,458,066.

For example, oligonucleotides may be prepared using an automated, solid-phase procedure based on the phosphoramidite approach. In such a method, each nucleotide is individually added to the 5′-end of the growing oligonucleotide chain, which is attached at the 3′-end to a solid support. The added nucleotides are in the form of trivalent 3′-phosphoramidites that are protected from polymerization by a dimethoxytriyl (or DMT) group at the 5′-position. After base-induced phosphoramidite coupling, mild oxidation to give a pentavalent phosphotriester intermediate and DMT removal provides a new site for oligonucleotide elongation. The oligonucleotides are then cleaved off the solid support, and the phosphodiester and exocyclic amino groups are deprotected with ammonium hydroxide. These syntheses may be performed on oligo synthesizers such as those commercially available from Perkin Elmer/Applied Biosystems, Inc. (Foster City, Calif.), DuPont (Wilmington, Del.) or Milligen (Bedford, Mass.). Alternatively, oligonucleotides can be custom made and ordered from a variety of commercial sources well-known in the art, including, for example, the Midland Certified Reagent Company (Midland, Tex.), ExpressGen, Inc. (Chicago, Ill.), Operon Technologies, Inc. (Huntsville, Ala.), and many others.

Purification of the oligonucleotides of the invention, where necessary or desirable, may be carried out by any of a variety of methods well-known in the art. Purification of oligonucleotides is typically performed either by native acrylamide gel electrophoresis, by anion-exchange HPLC as described, for example, by J. D. Pearson and F. E. Regnier (J. Chrom., 1983, 255: 137-149) or by reverse phase HPLC (G. D. McFarland and P. N. Borer, Nucleic Acids Res., 1979, 7: 1067-1080).

The sequence of oligonucleotides can be verified using any suitable sequencing method including, but not limited to, chemical degradation (A. M. Maxam and W. Gilbert, Methods of Enzymology, 1980, 65: 499-560), matrix-assisted laser desorption ionization time-of-flight (MALDI-TOF) mass spectrometry (U. Pieles et al., Nucleic Acids Res., 1993, 21: 3191-3196), mass spectrometry following a combination of alkaline phosphatase and exonuclease digestions (H. Wu and H. Aboleneen, Anal. Biochem., 2001, 290: 347-352), and the like.

As already mentioned above, modified oligonucleotides may be prepared using any of several means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc), or charged linkages (e.g., phosphorothioates, phosphorodithioates, etc). Oligonucleotides may contain one or more additional covalently linked moieties, such as, for example, proteins (e.g., nucleases, toxins, antibodies, signal peptides, poly-L-lysine, etc), intercalators (e.g., acridine, psoralen, etc), chelators (e.g., metals, radioactive metals, iron, oxidative metals, etc), and alkylators. The oligonucleotide may also be derivatized by formation of a methyl or ethyl phosphotriester or an alkyl phosphoramidate linkage. Furthermore, the oligonucleotide sequences of the present invention may also be modified with a label.

In certain embodiments, the detection probes or amplification primers or both probes and primers are labeled with a detectable agent or moiety before being used in amplification/detection assays. In certain embodiments, the detection probes are labeled with a detectable agent. Preferably, a detectable agent is selected such that it generates a signal which can be measured and whose intensity is related (e.g., proportional) to the amount of amplification products in the sample being analyzed.

The association between the oligonucleotide and detectable agent can be covalent or non-covalent. Labeled detection probes can be prepared by incorporation of or conjugation to a detectable moiety. Labels can be attached directly to the nucleic acid sequence or indirectly (e.g., through a linker). Linkers or spacer arms of various lengths are known in the art and are commercially available, and can be selected to reduce steric hindrance, or to confer other useful or desired properties to the resulting labeled molecules (see, for example, E. S. Mansfield et al., Mol. Cell. Probes, 1995, 9: 145-156).

Methods for labeling nucleic acid molecules are well-known in the art. For a review of labeling protocols, label detection techniques, and recent developments in the field, see, for example, L. J. Kricka, Ann. Clin. Biochem. 2002, 39: 114-129; R. P. van Gijlswijk et al., Expert Rev. Mol. Diagn. 2001, 1: 81-91; and S. Joos et al., J. Biotechnol. 1994, 35: 135-153. Standard nucleic acid labeling methods include: incorporation of radioactive agents, direct attachments of fluorescent dyes (L. M. Smith et al., Nucl. Acids Res., 1985, 13: 2399-2412) or of enzymes (B. A. Connoly and O. Rider, Nucl. Acids. Res., 1985, 13: 4485-4502); chemical modifications of nucleic acid molecules making them detectable immunochemically or by other affinity reactions (T. R. Broker et al., Nucl. Acids Res. 1978, 5: 363-384; E. A. Bayer et al., Methods of Biochem. Analysis, 1980, 26: 1-45; R. Langer et al., Proc. Natl. Acad. Sci. USA, 1981, 78: 6633-6637; R. W. Richardson et al., Nucl. Acids Res. 1983, 11: 6167-6184; D. J. Brigati et al., Virol. 1983, 126: 32-50; P. Tchen et al., Proc. Natl. Acad. Sci. USA, 1984, 81: 3466-3470; J. E. Landegent et al., Exp. Cell Res. 1984, 15: 61-72; and A. H. Hopman et al., Exp. Cell Res. 1987, 169: 357-368); and enzyme-mediated labeling methods, such as random priming, nick translation, PCR and tailing with terminal transferase (for a review on enzymatic labeling, see, for example, J. Temsamani and S. Agrawal, Mol. Biotechnol. 1996, 5: 223-232). More recently developed nucleic acid labeling systems include, but are not limited to: ULS (Universal Linkage System), which is based on the reaction of mono-reactive cisplatin derivatives with the N7 position of guanine moieties in DNA (R. J. Heetebrij et al., Cytogenet. Cell. Genet. 1999, 87: 47-52), psoralen-biotin, which intercalates into nucleic acids and upon UV irradiation becomes covalently bonded to the nucleotide bases (C. Levenson et al., Methods Enzymol. 1990, 184: 577-583; and C. Pfannschmidt et al., Nucleic Acids Res. 1996, 24: 1702-1709), photoreactive azido derivatives (C. Neves et al., Bioconjugate Chem. 2000, 11: 51-55), and DNA alkylating agents (M. G. Sebestyen et al., Nat. Biotechnol. 1998, 16: 568-576).

Any of a wide variety of detectable agents can be used in the practice of the present invention. Suitable detectable agents include, but are not limited to, various ligands, radionuclides (such as, for example, .sup.32P, .sup.35S, .sup.3H, sup.14C, .sup.125I, .sup.131I, and the like); fluorescent dyes (for specific exemplary fluorescent dyes, see below); chemiluminescent agents (such as, for example, acridinium esters, stabilized dioxetanes, and the like); spectrally resolvable inorganic fluorescent semiconductor nanocrystals (i.e., quantum dots), metal nanoparticles (e.g., gold, silver, copper and platinum) or nanoclusters; enzymes (such as, for example, those used in an ELISA, i.e., horseradish peroxidase, beta-galactosidase, luciferase, alkaline phosphatase); colorimetric labels (such as, for example, dyes, colloidal gold, and the like); magnetic labels (such as, for example, Dynabeads™); and biotin, dioxigenin or other haptens and proteins for which antisera or monoclonal antibodies are available.

In certain embodiments, the inventive detection probes are fluorescently labeled. Numerous known fluorescent labeling moieties of a wide variety of chemical structures and physical characteristics are suitable for use in the practice of this invention. Suitable fluorescent dyes include, but are not limited to, fluorescein and fluorescein dyes (e.g., fluorescein isothiocyanine or FITC, naphthofluorescein, 4′,5′-dichloro-2′,7′-dimethoxy-fluorescein, 6 carboxyfluorescein or FAM), carbocyanine, merocyanine, styryl dyes, oxonol dyes, phycoerythrin, erythrosin, eosin, rhodamine dyes (e.g., carboxytetramethylrhodamine or TAMRA, carboxyrhodamine 6G, carboxy-X-rhodamine (ROX), lissamine rhodamine B, rhodamine 6G, rhodamine Green, rhodamine Red, tetramethylrhodamine or TMR), coumarin and coumarin dyes (e.g., methoxycoumarin, dialkylaminocoumarin, hydroxycoumarin and aminomethylcoumarin or AMCA), Oregon Green Dyes (e.g., Oregon Green 488, Oregon Green 500, Oregon Green 514), Texas Red, Texas Red-X, Spectrum Red™, Spectrum Green™, cyanine dyes (e.g., Cy-3™, Cy-5™, Cy-3.5™, Cy-5.5™), Alexa Fluor dyes (e.g., Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), IRDyes (e.g., IRD40, IRD 700, IRD 800), and the like. For more examples of suitable fluorescent dyes and methods for linking or incorporating fluorescent dyes to nucleic acid molecules see, for example, “The Handbook of Fluorescent Probes and Research Products”, 9th Ed., Molecular Probes, Inc., Eugene, Oreg. Fluorescent dyes as well as labeling kits are commercially available from, for example, Amersham Biosciences, Inc. (Piscataway, N.J.), Molecular Probes Inc. (Eugene, Oreg.), and New England Biolabs Inc. (Berverly, Mass.).

As mentioned, identification of M17 may be carried out using an amplification reaction.

As used herein, the term “amplification” refers to a process that increases the representation of a population of specific nucleic acid sequences in a sample by producing multiple (i.e., at least 2) copies of the desired sequences. Methods for nucleic acid amplification are known in the art and include, but are not limited to, polymerase chain reaction (PCR) and ligase chain reaction (LCR). In a typical PCR amplification reaction, a nucleic acid sequence of interest is often amplified at least fifty thousand fold in amount over its amount in the starting sample. A “copy” or “amplicon” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable but not complementary to the template), and/or sequence errors that occur during amplification.

A typical amplification reaction is carried out by contacting a forward and reverse primer (a primer pair) to the sample DNA together with any additional amplification reaction reagents under conditions which allow amplification of the target sequence.

The terms “forward primer” and “forward amplification primer” are used herein interchangeably, and refer to a primer that hybridizes (or anneals) to the target (template strand). The terms “reverse primer” and “reverse amplification primer” are used herein interchangeably, and refer to a primer that hybridizes (or anneals) to the complementary target strand. The forward primer hybridizes with the target sequence 5′ with respect to the reverse primer.

The term “amplification conditions”, as used herein, refers to conditions that promote annealing and/or extension of primer sequences. Such conditions are well-known in the art and depend on the amplification method selected. Thus, for example, in a PCR reaction, amplification conditions generally comprise thermal cycling, i.e., cycling of the reaction mixture between two or more temperatures. In isothermal amplification reactions, amplification occurs without thermal cycling although an initial temperature increase may be required to initiate the reaction. Amplification conditions encompass all reaction conditions including, but not limited to, temperature and temperature cycling, buffer, salt, ionic strength, and pH, and the like.

As used herein, the term “amplification reaction reagents”, refers to reagents used in nucleic acid amplification reactions and may include, but are not limited to, buffers, reagents, enzymes having reverse transcriptase and/or polymerase activity or exonuclease activity, enzyme cofactors such as magnesium or manganese, salts, nicotinamide adenine dinuclease (NAD) and deoxynucleoside triphosphates (dNTPs), such as deoxyadenosine triphospate, deoxyguanosine triphosphate, deoxycytidine triphosphate and thymidine triphosphate. Amplification reaction reagents may readily be selected by one skilled in the art depending on the amplification method used.

According to this aspect of the present invention, the amplifying may be effected using techniques such as polymerase chain reaction (PCR), which includes, but is not limited to Allele-specific PCR, Assembly PCR or Polymerase Cycling Assembly (PCA), Asymmetric PCR, Helicase-dependent amplification, Hot-start PCR, Intersequence-specific PCR (ISSR), Inverse PCR, Ligation-mediated PCR, Methylation-specific PCR (MSP), Miniprimer PCR, Multiplex Ligation-dependent Probe Amplification, Multiplex-PCR, Nested PCR, Overlap-extension PCR, Quantitative PCR (Q-PCR), Reverse Transcription PCR (RT-PCR), Solid Phase PCR: encompasses multiple meanings, including Polony Amplification (where PCR colonies are derived in a gel matrix, for example), Bridge PCR (primers are covalently linked to a solid-support surface), conventional Solid Phase PCR (where Asymmetric PCR is applied in the presence of solid support bearing primer with sequence matching one of the aqueous primers) and Enhanced Solid Phase PCR (where conventional Solid Phase PCR can be improved by employing high Tm and nested solid support primer with optional application of a thermal ‘step’ to favour solid support priming), Thermal asymmetric interlaced PCR (TAIL-PCR), Touchdown PCR (Step-down PCR), PAN-AC and Universal Fast Walking.

The PCR (or polymerase chain reaction) technique is well-known in the art and has been disclosed, for example, in K. B. Mullis and F. A. Faloona, Methods Enzymol., 1987, 155: 350-355 and U.S. Pat. Nos. 4,683,202; 4,683,195; and 4,800,159 (each of which is incorporated herein by reference in its entirety). In its simplest form, PCR is an in vitro method for the enzymatic synthesis of specific DNA sequences, using two oligonucleotide primers that hybridize to opposite strands and flank the region of interest in the target DNA. A plurality of reaction cycles, each cycle comprising: a denaturation step, an annealing step, and a polymerization step, results in the exponential accumulation of a specific DNA fragment (“PCR Protocols: A Guide to Methods and Applications”, M. A. Innis (Ed.), 1990, Academic Press: New York; “PCR Strategies”, M. A. Innis (Ed.), 1995, Academic Press: New York; “Polymerase chain reaction: basic principles and automation in PCR: A Practical Approach”, McPherson et al. (Eds.), 1991, IRL Press: Oxford; R. K. Saiki et al., Nature, 1986, 324: 163-166). The termini of the amplified fragments are defined as the 5′ ends of the primers. Examples of DNA polymerases capable of producing amplification products in PCR reactions include, but are not limited to: E. coli DNA polymerase I, Klenow fragment of DNA polymerase I, T4 DNA polymerase, thermostable DNA polymerases isolated from Thermus aquaticus (Taq), available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs). RNA target sequences may be amplified by reverse transcribing the mRNA into cDNA, and then performing PCR (RT-PCR), as described above. Alternatively, a single enzyme may be used for both steps as described in U.S. Pat. No. 5,322,770.

The duration and temperature of each step of a PCR cycle, as well as the number of cycles, are generally adjusted according to the stringency requirements in effect. Annealing temperature and timing are determined both by the efficiency with which a primer is expected to anneal to a template and the degree of mismatch that is to be tolerated. The ability to optimize the reaction cycle conditions is well within the knowledge of one of ordinary skill in the art. Although the number of reaction cycles may vary depending on the detection analysis being performed, it usually is at least 15, more usually at least 20, and may be as high as 60 or higher. However, in many situations, the number of reaction cycles typically ranges from about 20 to about 40.

The denaturation step of a PCR cycle generally comprises heating the reaction mixture to an elevated temperature and maintaining the mixture at the elevated temperature for a period of time sufficient for any double-stranded or hybridized nucleic acid present in the reaction mixture to dissociate. For denaturation, the temperature of the reaction mixture is usually raised to, and maintained at, a temperature ranging from about 85° C. to about 100° C., usually from about 90° C. to about 98° C., and more usually from about 93° C. to about 96° C. for a period of time ranging from about 3 to about 120 seconds, usually from about 5 to about 30 seconds.

Following denaturation, the reaction mixture is subjected to conditions sufficient for primer annealing to template DNA present in the mixture. The temperature to which the reaction mixture is lowered to achieve these conditions is usually chosen to provide optimal efficiency and specificity, and generally ranges from about 50° C. to about ° C., usually from about 55° C. to about 70° C., and more usually from about 60° C. to about 68° C. Annealing conditions are generally maintained for a period of time ranging from about 15 seconds to about 30 minutes, usually from about 30 seconds to about 5 minutes.

Following annealing of primer to template DNA or during annealing of primer to template DNA, the reaction mixture is subjected to conditions sufficient to provide for polymerization of nucleotides to the primer's end in a such manner that the primer is extended in a 5′ to 3′ direction using the DNA to which it is hybridized as a template, (i.e., conditions sufficient for enzymatic production of primer extension product). To achieve primer extension conditions, the temperature of the reaction mixture is typically raised to a temperature ranging from about 65° C. to about 75° C., usually from about 67° C. to about 73° C., and maintained at that temperature for a period of time ranging from about 15 seconds to about 20 minutes, usually from about 30 seconds to about 5 minutes.

The above cycles of denaturation, annealing, and polymerization may be performed using an automated device typically known as a thermal cycler or thermocycler. Thermal cyclers that may be employed are described in U.S. Pat. Nos. 5,612,473; 5,602,756; 5,538,871; and 5,475,610 (each of which is incorporated herein by reference in its entirety). Thermal cyclers are commercially available, for example, from Perkin Elmer-Applied Biosystems (Norwalk, Conn.), BioRad (Hercules, Calif.), Roche Applied Science (Indianapolis, Ind.), and Stratagene (La Jolla, Calif.).

Amplification products obtained using primers of the present invention may be detected using agarose gel electrophoresis and visualization by ethidium bromide staining and exposure to ultraviolet (UV) light or by sequence analysis of the amplification product.

According to one embodiment, the amplification and quantification of the amplification product may be effected in real-time (qRT-PCR). Typically, QRT-PCR methods use double stranded DNA detecting molecules to measure the amount of amplified product in real time.

As used herein the phrase “double stranded DNA detecting molecule” refers to a double stranded DNA interacting molecule that produces a quantifiable signal (e.g., fluorescent signal). For example such a double stranded DNA detecting molecule can be a fluorescent dye that (1) interacts with a fragment of DNA or an amplicon and (2) emits at a different wavelength in the presence of an amplicon in duplex formation than in the presence of the amplicon in separation. A double stranded DNA detecting molecule can be a double stranded DNA intercalating detecting molecule or a primer-based double stranded DNA detecting molecule.

A double stranded DNA intercalating detecting molecule is not covalently linked to a primer, an amplicon or a nucleic acid template. The detecting molecule increases its emission in the presence of double stranded DNA and decreases its emission when duplex DNA unwinds. Examples include, but are not limited to, ethidium bromide, YO-PRO-1, Hoechst 33258, SYBR Gold, and SYBR Green I. Ethidium bromide is a fluorescent chemical that intercalates between base pairs in a double stranded DNA fragment and is commonly used to detect DNA following gel electrophoresis. When excited by ultraviolet light between 254 nm and 366 nm, it emits fluorescent light at 590 nm. The DNA-ethidium bromide complex produces about 50 times more fluorescence than ethidium bromide in the presence of single stranded DNA. SYBR Green I is excited at 497 nm and emits at 520 nm. The fluorescence intensity of SYBR Green I increases over 100 fold upon binding to double stranded DNA against single stranded DNA. An alternative to SYBR Green I is SYBR Gold introduced by Molecular Probes Inc. Similar to SYBR Green I, the fluorescence emission of SYBR Gold enhances in the presence of DNA in duplex and decreases when double stranded DNA unwinds. However, SYBR Gold's excitation peak is at 495 nm and the emission peak is at 537 nm. SYBR Gold reportedly appears more stable than SYBR Green I. Hoechst 33258 is a known bisbenzimide double stranded DNA detecting molecule that binds to the AT rich regions of DNA in duplex. Hoechst 33258 excites at 350 nm and emits at 450 nm. YO-PRO-1, exciting at 450 nm and emitting at 550 nm, has been reported to be a double stranded DNA specific detecting molecule. In a particular embodiment of the present invention, the double stranded DNA detecting molecule is SYBR Green I.

A primer-based double stranded DNA detecting molecule is covalently linked to a primer and either increases or decreases fluorescence emission when amplicons form a duplex structure. Increased fluorescence emission is observed when a primer-based double stranded DNA detecting molecule is attached close to the 3′ end of a primer and the primer terminal base is either dG or dC. The detecting molecule is quenched in the proximity of terminal dC-dG and dG-dC base pairs and dequenched as a result of duplex formation of the amplicon when the detecting molecule is located internally at least 6 nucleotides away from the ends of the primer. The dequenching results in a substantial increase in fluorescence emission. Examples of these type of detecting molecules include but are not limited to fluorescein (exciting at 488 nm and emitting at 530 nm), FAM (exciting at 494 nm and emitting at 518 nm), JOE (exciting at 527 and emitting at 548), HEX (exciting at 535 nm and emitting at 556 nm), TET (exciting at 521 nm and emitting at 536 nm), Alexa Fluor 594 (exciting at 590 nm and emitting at 615 nm), ROX (exciting at 575 nm and emitting at 602 nm), and TAMRA (exciting at 555 nm and emitting at 580 nm). In contrast, some primer-based double stranded DNA detecting molecules decrease their emission in the presence of double stranded DNA against single stranded DNA. Examples include, but are not limited to, rhodamine, and BODIPY-FI (exciting at 504 nm and emitting at 513 nm). These detecting molecules are usually covalently conjugated to a primer at the 5′ terminal dC or dG and emit less fluorescence when amplicons are in duplex. It is believed that the decrease of fluorescence upon the formation of duplex is due to the quenching of guanosine in the complementary strand in close proximity to the detecting molecule or the quenching of the terminal dC-dG base pairs.

According to one embodiment, the primer-based double stranded DNA detecting molecule is a 5′ nuclease probe. Such probes incorporate a fluorescent reporter molecule at either the 5′ or 3′ end of an oligonucleotide and a quencher at the opposite end. The first step of the amplification process involves heating to denature the double stranded DNA target molecule into a single stranded DNA. During the second step, a forward primer anneals to the target strand of the DNA and is extended by Taq polymerase. A reverse primer and a 5′ nuclease probe then anneal to this newly replicated strand.

In this embodiment, at least one of the primer pairs or 5′ nuclease probe should hybridize with a unique M17 sequence. The polymerase extends and cleaves the probe from the target strand. Upon cleavage, the reporter is no longer quenched by its proximity to the quencher and fluorescence is released. Each replication will result in the cleavage of a probe. As a result, the fluorescent signal will increase proportionally to the amount of amplification product.

The present invention contemplates various scenarios that would lead to the amplification of a unique M17 sequence:

According to the first scenario, both the forward primer and the reverse primer hybridize to a unique M17 sequence.

In the second scenario, only one of the primers of the primer pair hybridizes to a unique M17 sequence. The primer pair hybridizes to a non-unique M17 sequence. The primer pair may or may not be capable of hybridizing to non M17 sequences.

In the third scenario, neither of the primers hybridize to a unique M17 sequence, but both hybridize with a sequence that flanks the unique sequence. In such a scenario, the amplified sequence may be detected due to its unique size.

As shown in Example 2, herein below, primer pair (SEQ ID NOs: 37 and 38) and primer pair (SEQ ID NOs: 39 and 40) which hybridized with Fragment 33 of M17 were capable of distinguishing between M17 and other E. coli. In addition, primer pair (SEQ ID NO: 45 and 46) which hybridized with Fragment 44 was capable of distinguishing between M17 and other E. coli. The three primer pairs could also successfully identify M17 in a spiked fecal sample.

Below is a table (Table 9) listing additional primers contemplated by the present invention that may be used to identify the sequence as set forth in SEQ ID NO: 1 (Fragment 1). In the Table, and subsequent Tables 10-43, the primer pairs which generate a 60-200 bp product are typically used for real-time PCR, whereas the primers which generate a 300-500 bp product are typically used for standard PCR.

TABLE 9

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 62 | CCAGCT | 63 | GGTGTC | 4 | 1205 | 1201 |
| region | | CATCGT | | GATGCG | | | |
| | | GTTTTCC | | ATAAAA | | | |
| | | | | CC | | | |
| 60 bp-200 bp | 64 | AACCCT | 65 | GTTGCT | 78 | 269 | 191 |
| | | ATCGGT | | CAAGTG | | | |
| | | CAGGCT | | TCGCCA | | | |
| | | CT | | TA | | | |
| | 66 | CCTGAT | 67 | GGTGTC | 1016 | 1205 | 189 |
| | | GGCGGA | | GATGCG | | | |
| | | AAAGAA | | ATAAAA | | | |
| | | TA | | CC | | | |
| | 68 | TCCGGG | 69 | TGACAC | 207 | 305 | 98 |
| | | TTGATA | | CAACGC | | | |
| | | ACCATC | | CAATAA | | | |
| | | AT | | GA | | | |
| | 70 | TCGGTC | 71 | CGGAAG | 85 | 210 | 125 |
| | | AGGCTC | | CTGCGA | | | |
| | | TCTCAA | | ATTTTATT | | | |
| | | AT | | | | | |
| | 72 | CCTGAT | 73 | GCTTTTA | 1016 | 1111 | 95 |
| | | GGCGGA | | GGGCGC | | | |
| | | AAAGAA | | TGAGTTA | | | |
| | | TA | | | | | |
| | 74 | ATCGGT | 75 | GTTGCT | 84 | 269 | 185 |
| | | CAGGCT | | CAAGTG | | | |
| | | CTCTCA | | TCGCCA | | | |
| | | AA | | TA | | | |
| | 76 | TCCGGG | 77 | ACCACT | 207 | 703 | 496 |
| | | TTGATA | | CTGGTC | | | |
| | | ACCATC | | CTTCAT | | | |
| | | AT | | GC | | | |
| 300 bp-500 bp | 78 | TCGGTC | 79 | GATCCC | 85 | 586 | 501 |
| | | AGGCTC | | CATCAT | | | |
| | | TCTCAA | | GGAAAC | | | |
| | | AT | | AT | | | |
| | 80 | TATGGC | 81 | TTCCGG | 250 | 740 | 490 |
| | | GACACT | | GAAGAT | | | |
| | | TGAGCA | | AAATGG | | | |
| | | AC | | TG | | | |
| | 82 | TCTTATT | 83 | GCAGTA | 286 | 886 | 600 |
| | | GGCGTT | | TCCGGT | | | |
| | | GGTGTCA | | TTTTCAGC | | | |
| | 84 | AACCCT | 85 | GCGTAG | 78 | 470 | 392 |
| | | ATCGGT | | TGAATG | | | |
| | | CAGGCT | | CGGATG | | | |
| | | CT | | TA | | | |

Table 10 lists additional primers contemplated by the present invention that may be used to identify the sequence as set forth in SEQ ID NO: 2 (Fragment 2).

TABLE 10

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 86 | GGCGCTC | 87 | GCGGAA | −19 | 186 | 205 |
| region | | ATCGTAT | | TAACGA | | | |
| | | TGTGTA | | GTCCAC | | | |
| | | | | AT | | | |
| 60 bp-200 bp | 88 | AGAGCCT | 89 | ACATTTT | 21 | 170 | 149 |
| | | CGAAGAT | | GCTGTG | | | |
| | | GTTTGC | | GACCTTG | | | |
| | 90 | GCCTCGA | 91 | CGGGCA | 24 | 148 | 124 |
| | | AGATGTT | | TAATGA | | | |
| | | TGCTCT | | CCTTTTTC | | | |
| | 92 | AGAGCCT | 93 | CTGTAG | 21 | 119 | 98 |
| | | CGAAGAT | | GCCAGT | | | |
| | | GTTTGC | | GAGCGT | | | |
| | | | | TT | | | |

Table 11 lists additional primers contemplated by the present invention that may be used to identify the sequence as set forth in SEQ ID NO: 3 (Fragment 3).

TABLE 11

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 94 | CGCAGAC | 95 | GAGGGG | −25 | 1718 | 1743 |
| region | | CTACAGG | | AGGCAA | | | |
| | | AAGCAT | | AAGAAA | | | |
| | | | | AC | | | |
| 60 bp-200 bp | 96 | CCGGAAA | 97 | CAATTC | 1030 | 1133 | 103 |
| | | AATTAGC | | TCCGGC | | | |
| | | GTTGAA | | ATCAAG | | | |
| | | | | TT | | | |
| | 98 | TGTGGTT | 99 | TTGGCA | 1286 | 1401 | 115 |
| | | GGACTCA | | CTAATC | | | |
| | | TGCAAT | | GCCTAA | | | |
| | | | | CC | | | |
| | 100 | CAAAAGG | 101 | AGCCCA | 1151 | 1248 | 97 |
| | | AGGGCGA | | ATGCTG | | | |
| | | TAATGA | | ACTTGA | | | |
| | | | | AC | | | |
| | 102 | CGGGTGT | 103 | GAGTGG | 1258 | 1363 | 105 |
| | | TGTCCTA | | TCATTG | | | |
| | | ACTGCT | | GCCTCA | | | |
| | | | | TT | | | |
| 300 bp-500 bp | 104 | ATACCGC | 105 | GAGTGG | 770 | 1363 | 593 |
| | | CCAATAG | | TCATTG | | | |
| | | GGAAAG | | GCCTCA | | | |
| | | | | TT | | | |
| | 106 | ATACCGC | 107 | TCATTAT | 770 | 1170 | 400 |
| | | CCAATAG | | CGCCCT | | | |
| | | GGAAAG | | CCTTTTG | | | |
| | 108 | GGGGAAA | 109 | GGGCTT | 999 | 1506 | 507 |
| | | TAACGGG | | GATCAT | | | |
| | | AAAAGA | | TTGTGCTT | | | |
| | 110 | GCGATAA | 111 | ATTGCA | 817 | 1305 | 488 |
| | | CTGGGCA | | TGAGTC | | | |
| | | AATGAT | | CAACCA | | | |
| | | | | CA | | | |

Table 12 lists additional primers contemplated by the present invention that may be used to identify the sequence as set forth in SEQ ID NO: 4 (Fragment 4).

TABLE 12

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 112 | TCTTTCCG | 113 | CAGACC | −26 | 215 | 241 |
| region | | ATGGCTC | | CGTTTTG | | | |
| | | AGTCT | | CAGAGAT | | | |
| | 114 | GATGGCT | 115 | TTGAGG | −19 | 239 | 258 |
| | | CAGTCTG | | CTCCCG | | | |
| | | GAAAGG | | TAACAT | | | |
| | | | | TC | | | |
| 60 bp-200 bp | 116 | GGAATGG | 117 | CAGACC | 114 | 215 | 101 |
| | | TGCTGTTT | | CGTTTTG | | | |
| | | CCATT | | CAGAGAT | | | |
| | 118 | GCACGTG | 119 | AAACAG | 29 | 128 | 99 |
| | | TCACACT | | CACCAT | | | |
| | | GAAAAA | | TCCACA | | | |
| | | | | CA | | | |
| | 120 | AAAACTC | 121 | CGTTAA | 45 | 137 | 92 |
| | | CAGCTGG | | TGGAAA | | | |
| | | GATGG | | CAGCAC | | | |
| | | | | CA | | | |
| | 122 | CGTAGGT | 123 | GAACCG | 4 | 176 | 172 |
| | | AAAGGTC | | AGCCCA | | | |
| | | TGGATGG | | TTGGTA | | | |
| | | | | CT | | | |
| | 124 | GCTGGGA | 125 | GAACCG | 54 | 176 | 122 |
| | | TGGTGAT | | AGCCCA | | | |
| | | GTCAAT | | TTGGTA | | | |
| | | | | CT | | | |
| | 126 | TGCTGTTT | 127 | GAACCG | 85 | 176 | 91 |
| | | TTCTGAC | | AGCCCA | | | |
| | | GGATG | | TTGGTA | | | |
| | | | | CT | | | |
| | 128 | AAAACTC | 129 | AAACAG | 45 | 128 | 83 |
| | | CAGCTGG | | CACCAT | | | |
| | | GATGG | | TCCACA | | | |
| | | | | CA | | | |

Table 13 lists additional primers contemplated by the present invention that may be used to identify the sequence as set forth in SEQ ID NO: 5 (Fragment 5).

TABLE 13

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 130 | AGAGTCC | 131 | ACTTGC | −31 | 432 | 463 |
| region | | GTCTCCC | | CCAGGT | | | |
| | | ATAGCC | | TCTTGA | | | |
| | | | | AA | | | |
| | 132 | GTCCGTC | 133 | ACTTGC | −28 | 432 | 460 |
| | | TCCCATA | | CCAGGT | | | |
| | | GCCTTA | | TCTTGA | | | |
| | | | | AA | | | |
| | 134 | AAGTGTG | 135 | TATTAA | 210 | 327 | 117 |
| | | CCATTGC | | GGCGCC | | | |
| | | CTTTCT | | ACAACT | | | |
| | | | | GG | | | |
| 60 bp-200 bp | 136 | CAACTGG | 137 | GAAAAA | 153 | 251 | 98 |
| | | TACTGAG | | GAGCGG | | | |
| | | CGTTCG | | GTGAAC | | | |
| | | | | AA | | | |
| | 138 | GTTAATA | 139 | CTATCCT | 96 | 187 | 91 |
| | | TCGCGCG | | TGACCC | | | |
| | | TCCATC | | GACGAAC | | | |
| | 140 | CCATTGC | 141 | TATTAA | 217 | 327 | 110 |
| | | CTTTCTGC | | GGCGCC | | | |
| | | TTGTT | | ACAACT | | | |
| | | | | GG | | | |
| 300 bp-500 bp | 142 | TTGCTTCA | 143 | CGCAAT | 8 | 411 | 403 |
| | | TCATCGC | | TAGTGA | | | |
| | | CATT | | CCAGAT | | | |
| | | | | CG | | | |
| | 144 | CAACTGG | 145 | ACTTGC | 153 | 432 | 279 |
| | | TACTGAG | | CCAGGT | | | |
| | | CGTTCG | | TCTTGA | | | |
| | | | | AA | | | |

Table 14 lists additional primers contemplated by the present invention that may be used to identify the sequence as set forth in SEQ ID NO: 6 (Fragment 6).

TABLE 14

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 146 | ATGGGCA | 147 | AATCGT | −43 | 579 | 622 |
| region | | TCACGCA | | GTCCAG | | | |
| | | AGATA | | CCATTTTG | | | |
| 60 bp-200 bp | 148 | GTTGTGG | 149 | GATATC | 264 | 374 | 110 |
| | | AGCAGCT | | CTGCGG | | | |
| | | TGAACA | | ACGCTC | | | |
| | | | | TA | | | |
| | 150 | GTTGTGG | 151 | GCTCTA | 264 | 360 | 96 |
| | | AGCAGCT | | TCCCTG | | | |
| | | TGAACA | | CTGAAT | | | |
| | | | | GC | | | |
| 300 bp-500 bp | 152 | CCAACTA | 153 | CCGAAT | 49 | 512 | 463 |
| | | CCCACCC | | CGTTGA | | | |
| | | TGTGTC | | CTCGTA | | | |
| | | | | TG | | | |
| | 154 | CCAACTA | 155 | CAACCG | 49 | 435 | 386 |
| | | CCCACCC | | CTCGAA | | | |
| | | TGTGTC | | CACCTT | | | |
| | | | | AG | | | |

Table 15 lists additional primers contemplated by the present invention that may be used to identify the sequence as set forth in SEQ ID NO: 7 (Fragment 7).

TABLE 15

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 156 | CCACGGT | 157 | CTTCGG | −44 | 1366 | 1410 |
| region | | ATATCCC | | GATTCA | | | |
| | | TCGGTA | | TGGTTA | | | |
| | | | | GC | | | |
| | 158 | GGCAGTC | 159 | ATTGGG | 759 | 1389 | 630 |
| | | TTTCTGGC | | TGAGCC | | | |
| | | ATAGG | | TGATTG | | | |
| | | | | AA | | | |
| 60 bp-200 bp | 160 | GGTGCTA | 161 | GATTTC | 428 | 551 | 123 |
| | | GACTCTG | | CCACGC | | | |
| | | GGCTTG | | TGTCAC | | | |
| | | | | TT | | | |
| | 162 | TGATCAG | 163 | AAGCCC | 346 | 446 | 100 |
| | | TGATTGC | | AGAGTC | | | |
| | | GTGACA | | TAGCAC | | | |
| | | | | CA | | | |
| 300 bp-500 bp | 164 | GGTGCTA | 165 | AGCATA | 428 | 926 | 498 |
| | | GACTCTG | | CCCAAA | | | |
| | | GGCTTG | | ATGGCA | | | |
| | | | | AC | | | |
| | 166 | AGGTCGA | 167 | GCAAAG | 188 | 784 | 596 |
| | | TCTACGC | | CCTATG | | | |
| | | GAAAAA | | CCAGAA | | | |
| | | | | AG | | | |
| | 168 | ATCAGAA | 169 | TCCAGG | 456 | 854 | 398 |
| | | CCCGACG | | CTTCGA | | | |
| | | ACAAAG | | GGAGAG | | | |
| | | | | TA | | | |

Table 16 lists additional primers contemplated by the present invention that may be used to identify the sequence as set forth in SEQ ID NO: 8 (Fragment 8).

TABLE 16

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 170 | AACAAGTC | 171 | GTCAGG | −34 | 827 | 861 |
| region | | CAGTGCAT | | ACGATT | | | |
| | | CACG | | TCGTTG | | | |
| | | | | GT | | | |
| 60 bp-200 bp | 172 | CAGGTATG | 173 | ATGAAC | 10 | 128 | 118 |
| | | GCAAGGAC | | CCTTGC | | | |
| | | GATT | | GAATCA | | | |
| | | | | AG | | | |
| | 174 | CTTGATTC | 175 | GGAGTT | 109 | 208 | 99 |
| | | GCAAGGGT | | ACGCGA | | | |
| | | TCAT | | GTTGCTTT | | | |
| 300 bp-500 bp | 176 | TTCCATGA | 177 | ATGTCC | 29 | 537 | 508 |
| | | CTCGTCAG | | CATATA | | | |
| | | CAAG | | GCCCGT | | | |
| | | | | TG | | | |
| | 178 | CTTGATTC | 179 | CTTTCTC | 109 | 713 | 604 |
| | | GCAAGGGT | | GGACGA | | | |
| | | TCAT | | ACGATTT | | | |
| | 180 | TTGCCAAC | 181 | TCGAAA | 250 | 653 | 403 |
| | | GATACAAA | | TTGACC | | | |
| | | TCCA | | CGAAAC | | | |
| | | | | TC | | | |

Table 17 lists additional primers contemplated by the present invention that may be used to identify the sequence as set forth in SEQ ID NO: 9 (Fragment 9).

TABLE 17

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 182 | TTTTtGGAA | 183 | GCGTTG | −24 | 377 | 401 |
| region | | TTGGCAGC | | TGTTCTT | | | |
| | | ATC | | CTGTGGA | | | |
| 60 bp-200 bp | 184 | GCTTTGGC | 185 | AATTGA | 150 | 271 | 121 |
| | | TTAAGGGC | | GCGTGA | | | |
| | | TTTT | | GGTTTTCG | | | |
| | 186 | CCACATGA | 187 | GGGCGC | 39 | 139 | 100 |
| | | AAGAGACG | | TATTGA | | | |
| | | GTCA | | TACTCA | | | |
| | | | | GG | | | |
| 300 bp-500 bp | 188 | AAGCCTGG | 189 | ACCTGG | 15 | 331 | 316 |
| | | CCTGTACG | | TTTCAA | | | |
| | | TTTA | | AGGGTT | | | |
| | | | | GG | | | |

Table 18 lists additional primers contemplated by the present invention that may be used to identify the sequence as set forth in SEQ ID NO: 10 (Fragment 10).

TABLE 18

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 190 | GCAGGGA | 191 | AACAGG | 26 | 576 | 550 |
| region | | TCCAGCT | | GTTTGG | | | |
| | | AATTGA | | GACATT | | | |
| | | | | TTT | | | |
| 60 bp-200 bp | 192 | CGATTCG | 193 | TTTTGCC | −34 | 62 | 96 |
| | | CAAGAAT | | TCTGTC | | | |
| | | CTGGA | | ACCATCA | | | |
| | 194 | AAAATGC | 195 | GCGGAT | 21 | 117 | 96 |
| | | AGGGATC | | AAGAAA | | | |
| | | CAGCTA | | AGACAA | | | |
| | | | | TAGCC | | | |
| | 196 | CGAGCTG | 197 | GCACTG | 317 | 419 | 102 |
| | | ATAATAA | | CAGAGC | | | |
| | | ATTATGG | | CAGAGA | | | |
| | | AACC | | TA | | | |
| | 198 | CGAGCTG | 199 | ATCTCTC | 317 | 439 | 122 |
| | | ATAATAA | | GCGGGT | | | |
| | | ATTATGG | | AGTTGAG | | | |
| | | AACC | | | | | |
| 300 bp-500 bp | 200 | TTGATGG | 201 | ATCTCTC | 42 | 439 | 397 |
| | | TGACAGA | | GCGGGT | | | |
| | | GGCAAA | | AGTTGAG | | | |

Table 19 lists additional primers contemplated by the present invention that may be used to identify the sequence as set forth in SEQ ID NO: 11 (Fragment 11).

TABLE 19

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 202 | CTCGCTTT | 203 | GAGATG | −48 | 497 | 545 |
| region | | TCCCAAG | | AGTTTC | | | |
| | | TGAAT | | GGGAGC | | | |
| | | | | AG | | | |
| 60 bp-200 bp | 204 | GGAAACA | 205 | CACCCC | 9 | 114 | 105 |
| | | AACCGAC | | ACCAGA | | | |
| | | TGGAAA | | ACCATA | | | |
| | | | | AA | | | |
| | 206 | GAATACT | 207 | TGGGCA | 306 | 392 | 86 |
| | | GATGCGG | | ATGATT | | | |
| | | CAGTCC | | GTTTGT | | | |
| | | | | GT | | | |
| 300 bp-500 bp | 208 | GGCTTAT | 209 | TGGGCA | 42 | 392 | 350 |
| | | GGGAAAG | | ATGATT | | | |
| | | CACTCA | | GTTTGT | | | |
| | | | | GT | | | |

Table 20 lists additional primers contemplated by the present invention that may be used to identify the sequence as set forth in SEQ ID NO: 12 (Fragment 12).

TABLE 20

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 210 | CGATTGT | 211 | AGCTAG | −34 | 464 | 498 |
| region | | GGCTGAA | | ATCGCC | | | |
| | | ATTGAA | | ACTGGA | | | |
| | | | | AA | | | |
| 60 bp-200 bp | 212 | GCGACAA | 213 | TTGCTC | 255 | 356 | 101 |
| | | CATCAGA | | ATGGCG | | | |
| | | AAACGA | | AAATAA | | | |
| | | | | CA | | | |
| | 214 | GCCCACT | 215 | GCTAGA | 372 | 463 | 91 |
| | | TTTTCCAG | | TCGCCA | | | |
| | | TCAAA | | CTGGAA | | | |
| | | | | AC | | | |
| | 216 | AGGTGCA | 217 | TTTGCC | −7 | 80 | 87 |
| | | GAAATGA | | AATATT | | | |
| | | GCGAGT | | CCCCAG | | | |
| | | | | AG | | | |
| 300 bp-500 bp | 218 | GCGACAA | 219 | AGCTAG | 255 | 464 | 209 |
| | | CATCAGA | | ATCGCC | | | |
| | | AAACGA | | ACTGGA | | | |
| | | | | AA | | | |
| | 220 | AGGTGCA | 221 | GGTTTT | −7 | 292 | 299 |
| | | GAAATGA | | ACGACA | | | |
| | | GCGAGT | | TCCTCAT | | | |
| | | | | CG | | | |

Table 21 lists additional primers contemplated by the present invention that may be used to identify the sequence as set forth in SEQ ID NO: 13 (Fragment 13).

TABLE 21

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 222 | ACTTTGG | 223 | TTGGTC | −17 | 443 | 460 |
| region | | GAGAAGT | | AGGGTG | | | |
| | | GGCTCA | | TCGAAT | | | |
| | | | | TT | | | |
| 60 bp-200 bp | 224 | TGCAAAA | 225 | GGCGGA | 71 | 169 | 98 |
| | | CAGAGCA | | TATTCCC | | | |
| | | GGAAAA | | AATCAAT | | | |
| | 226 | GTGGAAC | 227 | CGGCAT | 206 | 339 | 133 |
| | | AAATGGC | | TTTTGCT | | | |
| | | GATGTA | | CCTTTAG | | | |
| | 228 | TGCAAAA | 229 | CACCGA | 71 | 208 | 137 |
| | | CAGAGCA | | TACAAT | | | |
| | | GGAAAA | | CTGCTT | | | |
| | | | | GC | | | |
| 300 bp-500 bp | 230 | ATCTGCT | 231 | GAGTGG | 65 | 390 | 325 |
| | | GCAAAAC | | CGGTAC | | | |
| | | AGAGCA | | AGGGATT | | | |

Table 22 lists additional primers contemplated by the present invention that may be used to identify the sequence as set forth in SEQ ID NO: 14 (Fragment 14).

TABLE 22

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 232 | GCAGGGC | 233 | GAGGGC | −29 | 669 | 698 |
| region | | TTGGAGA | | GGGATT | | | |
| | | TCATT | | TCTACTTC | | | |
| 60 bp-200 bp | 234 | AGCCAGG | 235 | CATGCC | 538 | 637 | 99 |
| | | CAAAAGG | | AATCCA | | | |
| | | ATACAA | | TCACTG | | | |
| | | | | AA | | | |
| | 236 | CATTCGT | 237 | TTTCAA | 153 | 254 | 101 |
| | | GCAAGCA | | GACCTG | | | |
| | | AGAGAA | | CACCTT | | | |
| | | | | CA | | | |
| | 238 | AATTATG | 239 | CCTCTA | 126 | 213 | 87 |
| | | GGAGCAA | | GGATCC | | | |
| | | GGCAGA | | GGCTCA | | | |
| | | | | AT | | | |
| 300 bp-500 bp | 240 | TGAAGGT | 241 | GGATTC | 235 | 688 | 453 |
| | | GCAGGTC | | CTCAGC | | | |
| | | TTGAAA | | GCTAAC | | | |
| | | | | TG | | | |

Table 23 lists additional primers contemplated by the present invention that may be used to identify the sequence as set forth in SEQ ID NO: 15 (Fragment 15).

TABLE 23

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 242 | GCGCAAC | 243 | CCGCCA | −42 | 846 | 888 |
| region | | GGATAAG | | TATCGTT | | | |
| | | GATAAA | | GTTATA | | | |
| | | | | CG | | | |
| 60 bp-200 bp | 244 | GGCATTA | 245 | CTGTTTT | 580 | 695 | 115 |
| | | ACCCGTC | | CGGAAA | | | |
| | | TTCTGA | | TGCCTGT | | | |
| | 246 | GGGCAGA | 247 | TGCTGT | 132 | 235 | 103 |
| | | CTATCAG | | GCGTAA | | | |
| | | GCAGAG | | TTGTGG | | | |
| | | | | AT | | | |
| 300 bp-500 bp | 248 | GGGCAGA | 249 | CGAGTT | 132 | 620 | 488 |
| | | CTATCAG | | TGATAC | | | |
| | | GCAGAG | | GCCCTT | | | |
| | | | | CT | | | |
| | 250 | GGAGGTT | 251 | GCTCGT | 53 | 659 | 606 |
| | | CAGCAAC | | CAACAC | | | |
| | | AACGAT | | TTCCTTCC | | | |
| | 252 | CAATTAC | 253 | CGAGTT | 221 | 620 | 399 |
| | | GCACAGC | | TGATAC | | | |
| | | AACTGG | | GCCCTT | | | |
| | | | | CT | | | |

Table 24 lists additional primers contemplated by the present invention that may be used to identify the sequence as set forth in SEQ ID NO: 16 (Fragment 16).

TABLE 24

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 254 | ACGcTCCC | 255 | GGATGT | −14 | 387 | 401 |
| region | | CCTGTAA | | GATCCA | | | |
| | | AACA | | TCTGGT | | | |
| | | | | GA | | | |
| 60 bp-200 bp | 256 | GAGGCAT | 257 | ATGGAT | 24 | 122 | 98 |
| | | AAACCCA | | TTGCCT | | | |
| | | TGCTGT | | GTCTGA | | | |
| | | | | CC | | | |
| | 258 | TGGCATA | 259 | AGACAA | 201 | 318 | 117 |
| | | ACCGATG | | CGGGCT | | | |
| | | AACAGA | | GTAGCA | | | |
| | | | | TT | | | |
| 300 bp-500 bp | 260 | CGTACCT | 261 | GGATGT | 16 | 387 | 371 |
| | | GGAGGCA | | GATCCA | | | |
| | | TAAACC | | TCTGGT | | | |
| | | | | GA | | | |

Table 25 lists additional primers contemplated by the present invention that may be used to identify the sequence as set forth in SEQ ID NO: 17 (Fragment 17).

TABLE 25

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 262 | AACAGTT | 263 | ACTAAT | −46 | 316 | 362 |
| region | | CCTGAGT | | GCGAGT | | | |
| | | GTAATCA | | GCTTGC | | | |
| | | CCA | | TG | | | |
| 60 bp-200 bp | 264 | ATGTTCC | 265 | CCAGCA | 0 | 98 | 98 |
| | | GATTCGC | | ATCGTTT | | | |
| | | AATGTT | | GTGTTTG | | | |
| | 266 | ATTCGCC | 267 | GCCAAT | 64 | 162 | 98 |
| | | CGAACAT | | ATGACT | | | |
| | | ACAAAC | | CACCCA | | | |
| | | | | CA | | | |
| | 268 | AATATAC | 269 | CCGGGT | 209 | 283 | 74 |
| | | CCGCTGG | | ATGGAA | | | |
| | | TCCAAA | | ATCACT | | | |
| | | | | TG | | | |
| 300 bp-500 bp | 270 | ATGTTCC | 271 | ACTAAT | 0 | 316 | 316 |
| | | GATTCGC | | GCGAGT | | | |
| | | AATGTT | | GCTTGC | | | |
| | | | | TG | | | |
| | 272 | ATTCGCC | 273 | ACTAAT | 64 | 316 | 252 |
| | | CGAACAT | | GCGAGT | | | |
| | | ACAAAC | | GCTTGC | | | |
| | | | | TG | | | |

Table 26 lists additional primers contemplated by the present invention that may be used to identify the sequence as set forth in SEQ ID NO: 18 (Fragment 18).

TABLE 26

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 274 | GTACTTTC | 275 | CAATGA | −27 | 510 | 537 |
| region | | ACCTCCC | | CATGGA | | | |
| | | GACCA | | CGCTGG | | | |
| | | | | TA | | | |
| 60 bp-200 bp | 276 | GCACCCT | 277 | GCATCG | 299 | 399 | 100 |
| | | ATCCATT | | CCAGCG | | | |
| | | CACCAT | | TTATTATT | | | |
| | 278 | TGCCAGT | 279 | AAAGCC | 442 | 547 | 105 |
| | | CAGTGAG | | ATTAAG | | | |
| | | CTATGG | | GCGTAG | | | |
| | | | | GG | | | |
| | 280 | AATAATA | 281 | TGCCAT | 380 | 463 | 83 |
| | | ACGCTGG | | AGCTCA | | | |
| | | CGATGC | | CTGACT | | | |
| | | | | GG | | | |
| | 282 | GGTAGCA | 283 | CGGAAT | 132 | 236 | 104 |
| | | CCAGTCA | | TGAAAA | | | |
| | | GGCTGT | | CCTCTG | | | |
| | | | | CT | | | |
| 300 bp-500 bp | 284 | TGAAAGT | 285 | GCATCG | 3 | 399 | 396 |
| | | TCGTTCA | | CCAGCG | | | |
| | | GCTTGC | | TTATTATT | | | |
| | 286 | GGTAGCA | 287 | GCCCAG | 132 | 431 | 299 |
| | | CCAGTCA | | TATGAT | | | |
| | | GGCTGT | | GTCCAG | | | |
| | | | | AAA | | | |

Table 27 lists additional primers contemplated by the present invention that may be used to identify the sequence as set forth in SEQ ID NO: 19 (Fragment 19).

TABLE 27

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 288 | CATCCTG | 289 | ATAAAA | −19 | 194 | 213 |
| region | | GAACAGA | | CCAATC | | | |
| | | CTGGGTA | | GGCCCA | | | |
| | | | | AC | | | |
| 60 bp-200 bp | 290 | CATGGCA | 291 | CGTAAC | 50 | 140 | 90 |
| | | ACTTACG | | AGGGAG | | | |
| | | GCATTA | | GAAAGA | | | |
| | | | | CG | | | |
| | 292 | ACGCATG | 293 | CGATAA | 69 | 165 | 96 |
| | | GGAGAAG | | TGCTGC | | | |
| | | AAAGAG | | AAGCAA | | | |
| | | | | AC | | | |
| | 294 | ACAGCAA | 295 | ATAAAA | 111 | 194 | 83 |
| | | CTGCGTC | | CCAATC | | | |
| | | TTTCCT | | GGCCCA | | | |
| | | | | AC | | | |

Table 28 lists additional primers contemplated by the present invention that may be used to identify the sequence as set forth in SEQ ID NO: 20 (Fragment 20).

TABLE 28

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 296 | CCAATAG | 297 | GCCAGA | −41 | 421 | 462 |
| region | | GCATCGT | | TGTTCTA | | | |
| | | CACCTC | | TCCCCA | | | |
| | | | | GT | | | |
| 60 bp-200 bp | 298 | AATGGTT | 299 | TGGCAT | 150 | 248 | 98 |
| | | GCGGTAA | | TTGCATT | | | |
| | | ATCGAC | | AGGTTGA | | | |
| | 300 | ATTCTGG | 301 | TGGCAT | 138 | 248 | 110 |
| | | GACCAAA | | TTGCATT | | | |
| | | TGGTTG | | AGGTTGA | | | |
| | 302 | TACCTGA | 303 | AATGTC | 106 | 172 | 66 |
| | | ACTGCAA | | GATTTA | | | |
| | | CGAGGA | | CCGCAA | | | |
| | | | | CC | | | |
| 300 bp-500 bp | 304 | CGCTATC | 305 | CGCGTA | 16 | 345 | 329 |
| | | GCAGGAG | | GGGAAA | | | |
| | | TTTGTT | | CCAGAA | | | |
| | | | | TA | | | |
| | 306 | CGCTATC | 307 | ATCTTTG | 16 | 355 | 339 |
| | | GCAGGAG | | ACGCGC | | | |
| | | TTTGTT | | GTAGG | | | |
| | 308 | CGCTATC | 309 | ATGTTTC | 16 | 368 | 352 |
| | | GCAGGAG | | CCGCCC | | | |
| | | TTTGTT | | ATCTT | | | |

Table 29 lists additional primers contemplated by the present invention that may be used to identify the sequence as set forth in SEQ ID NO: 21 (Fragment 21).

TABLE 29

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 310 | CCATCAGT | 311 | CTCTGA | −20 | 1137 | 1157 |
| region | | TCCATGTT | | TGTATC | | | |
| | | ATGGATT | | CGGTGT | | | |
| | | | | GC | | | |
| | 312 | TACAATGA | 313 | ATGGCC | 451 | 1100 | 649 |
| | | ATGCCCTG | | AGTGAG | | | |
| | | TTGG | | CGTAAA | | | |
| | | | | AA | | | |
| | 314 | CCATCAGT | 315 | GGTGCA | −20 | 623 | 643 |
| | | TCCATGTT | | TTGATTC | | | |
| | | ATGGATT | | CACGTC | | | |
| 60 bp-200 bp | 316 | CCGTGGA | 317 | TGAGAA | 178 | 284 | 106 |
| | | CGAATAG | | GTTCCG | | | |
| | | AGCATT | | GGAGAG | | | |
| | | | | AA | | | |
| | 318 | AGTTGCTG | 319 | TTTTTGG | 544 | 628 | 84 |
| | | CTGACGA | | TGCATT | | | |
| | | CCTTC | | GATTCCA | | | |
| | 320 | CTGTGGTT | 321 | ATGGCC | 1016 | 1100 | 84 |
| | | GAGGTTTG | | AGTGAG | | | |
| | | AGCA | | CGTAAA | | | |
| | | | | AA | | | |
| | 322 | CCTCTAGT | 323 | TTTTTGG | 539 | 628 | 89 |
| | | TGCTGCTG | | TGCATT | | | |
| | | ACGA | | GATTCCA | | | |
| | 324 | CGGTTTCG | 325 | ATGGGT | 515 | 603 | 88 |
| | | ATACGCTC | | TTTGGA | | | |
| | | TTTT | | TCTGAA | | | |
| | | | | CG | | | |
| | 326 | GGTCTCGG | 327 | CCGGGC | 895 | 1005 | 110 |
| | | AAGATCG | | AAAAGT | | | |
| | | AGAAA | | ATCAAA | | | |
| | | | | AA | | | |
| 300 bp-500 bp | 328 | TGGAATCA | 329 | CCGGGC | 609 | 1005 | 396 |
| | | ATGCACC | | AAAAGT | | | |
| | | AAAAA | | ATCAAA | | | |
| | | | | AA | | | |
| | 330 | TTCTCTCC | 331 | AAATAC | 265 | 666 | 401 |
| | | CGGAACTT | | GGGGAA | | | |
| | | CTCA | | TTGTGT | | | |
| | | | | GG | | | |
| | 332 | GTGGAATC | 333 | CCGGGC | 608 | 1005 | 397 |
| | | AATGCAC | | AAAAGT | | | |
| | | CAAAA | | ATCAAA | | | |
| | | | | AA | | | |

Table 30 lists additional primers contemplated by the present invention that may be used to identify the sequence as set forth in SEQ ID NO: 22 (Fragment 22).

TABLE 30

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 334 | CCGTAAC | 335 | TTTTGTT | −29 | 591 | 620 |
| region | | GGTCAAA | | CACCAC | | | |
| | | AATCGT | | GACCTCA | | | |
| | 336 | AAAAATC | 337 | TTTTGTT | −18 | 591 | 609 |
| | | GTGGCGTT | | CACCAC | | | |
| | | GACAC | | GACCTCA | | | |
| 60 bp-200 bp | 338 | GTATCGAC | 339 | TGACTG | 36 | 111 | 75 |
| | | TGGTGGCA | | CCTTTCT | | | |
| | | TCTG | | CCCACTT | | | |
| | 340 | GGCATCTG | 341 | TGACTG | 48 | 111 | 63 |
| | | GAGATAC | | CCTTTCT | | | |
| | | GCTTT | | CCCACTT | | | |
| | 342 | TGCTTTTT | 343 | CAAACG | 447 | 508 | 61 |
| | | GATGATG | | GGGAAT | | | |
| | | GAAACA | | GTAGCA | | | |
| | | | | AT | | | |
| | 344 | TGCATTGC | 345 | AGCCAA | 241 | 332 | 91 |
| | | GGTTTTAA | | GCCAAT | | | |
| | | TCTTT | | TTATTTC | | | |
| | | | | AA | | | |
| | 346 | GTATCGAC | 347 | CAGCAT | 36 | 116 | 80 |
| | | TGGTGGCA | | GACTGC | | | |
| | | TCTG | | CTTTCTCC | | | |
| | 348 | AATGCATT | 349 | TTGAAT | 239 | 338 | 99 |
| | | GCGGTTTT | | AGCCAA | | | |
| | | AATCTT | | GCCAAT | | | |
| | | | | TT | | | |
| 300 bp-500 bp | 350 | CTGCAGC | 351 | CAAACG | 114 | 508 | 394 |
| | | ATTGACGA | | GGGAAT | | | |
| | | TTTGT | | GTAGCA | | | |
| | | | | AT | | | |
| | 352 | TGCAGCAT | 353 | TATCAC | 115 | 515 | 400 |
| | | TGACGATT | | CCAAAC | | | |
| | | TGTT | | GGGGAAT | | | |
| | 354 | CGGACAT | 355 | CAAACG | 192 | 508 | 316 |
| | | AAATATCT | | GGGAAT | | | |
| | | CAAAATG | | GTAGCA | | | |
| | | ACA | | AT | | | |

Table 31 lists additional primers contemplated by the present invention that may be used to identify the sequence as set forth in SEQ ID NO: 23 (Fragment 23).

TABLE 31

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 356 | GAGCGTCT | 357 | GTAGTG | −16 | 322 | 338 |
| region | | GCTCAAA | | GGTGAG | | | |
| | | CAGGT | | GGGCTGT | | | |
| | 358 | TCAAACA | 359 | GTAGTG | −6 | 322 | 328 |
| | | GGTTATCC | | GGTGAG | | | |
| | | GTCAGG | | GGGCTGT | | | |
| 60 bp-200 bp | 360 | TCCGTCAG | 361 | CAGAGC | 6 | 108 | 102 |
| | | GAAGAGG | | GTCAAG | | | |
| | | AAAAA | | CAACCT | | | |
| | | | | TT | | | |
| 300 bp-500 bp | 362 | TCCGTCAG | 363 | CCTTAC | 6 | 263 | 257 |
| | | GAAGAGG | | CTATTTC | | | |
| | | AAAAA | | CGCTGGT | | | |

Table 32 lists additional primers contemplated by the present invention that may be used to identify the sequence as set forth in SEQ ID NO: 24 (Fragment 24).

TABLE 32

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 364 | TTGAAAAC | 365 | CGTTGA | −11 | 1241 | 1252 |
| region | | CAGAGCC | | CCATAA | | | |
| | | TTGCT | | GAAAAC | | | |
| | | | | CTCA | | | |
| | 366 | AATGAAA | 367 | CGTTGA | 694 | 1241 | 547 |
| | | AAGGAAA | | CCATAA | | | |
| | | CGCCATA | | GAAAAC | | | |
| | | | | CTCA | | | |
| 60 bp-200 bp | 368 | TTTtCTGCT | 369 | GGCCCG | 205 | 347 | 142 |
| | | GCAAGCA | | TTTATCA | | | |
| | | CTTC | | GAAAGGT | | | |
| | 370 | AAAAGGG | 371 | TCCAAA | 528 | 619 | 91 |
| | | AAGGCCTT | | ATGGGA | | | |
| | | ATGATG | | AAGAAG | | | |
| | | | | AGG | | | |
| | 372 | TTTCATAG | 373 | AAAATG | 272 | 352 | 80 |
| | | GAAGTGG | | GCCCGT | | | |
| | | AGGTGGT | | TTATCA | | | |
| | | | | GA | | | |
| 300 bp-500 bp | 374 | TTTtCTGCT | 375 | CCAAAA | 205 | 618 | 413 |
| | | GCAAGCA | | TGGGAA | | | |
| | | CTTC | | AGAAGA | | | |
| | | | | GG | | | |
| | 376 | CCTTGCTT | 377 | GGCCCG | 2 | 347 | 345 |
| | | AGACCTGT | | TTTATCA | | | |
| | | GTCCA | | GAAAGGT | | | |
| | 378 | ACGTGGGT | 379 | AAAATG | 27 | 352 | 325 |
| | | TGGATCAT | | GCCCGT | | | |
| | | TGTT | | TTATCA | | | |
| | | | | GA | | | |

Table 33 lists additional primers contemplated by the present invention that may be used to identify the sequence as set forth in SEQ ID NO: 25 (Fragment 25).

TABLE 33

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 380 | GGGTCTCG | 381 | GGCTTT | −39 | 827 | 866 |
| region | | ATTTGATG | | ACCGTG | | | |
| | | ATTGA | | GTAGTA | | | |
| | | | | CTGG | | | |
| | 382 | TCTTGGTA | 383 | GGCTTT | −10 | 827 | 837 |
| | | GGGACGT | | ACCGTG | | | |
| | | GGTTT | | GTAGTA | | | |
| | | | | CTGG | | | |
| 60 bp-200 bp | 384 | CTTCATTT | 385 | TTGGGT | 299 | 395 | 96 |
| | | TGGCCTCT | | AGCCAC | | | |
| | | TTGC | | ATCCCTTA | | | |
| | 386 | GATGTGGC | 387 | CACCCA | 381 | 535 | 154 |
| | | TACCCAAG | | AGGACT | | | |
| | | CAAT | | GAAGGA | | | |
| | | | | AG | | | |
| | 388 | GGGCATG | 389 | ATTTCCC | 535 | 685 | 150 |
| | | GGCATACT | | CTCAAT | | | |
| | | TATCA | | TCCTTCG | | | |
| 300 bp-500 bp | 390 | TGTGCACC | 391 | ATTTCCC | 226 | 685 | 459 |
| | | TTAGTCGC | | CTCAAT | | | |
| | | TTTG | | TCCTTCG | | | |
| | 392 | CTTCATTT | 393 | TCTGAG | 299 | 767 | 468 |
| | | TGGCCTCT | | CGATTTT | | | |
| | | TTGC | | TCTTGA | | | |
| | | | | GC | | | |
| | 394 | ATGGCGGT | 395 | ATTTCCC | 343 | 685 | 342 |
| | | AGCTCATA | | CTCAAT | | | |
| | | CCTTT | | TCCTTCG | | | |

Table 34 lists additional primers contemplated by the present invention that may be used to identify the sequence as set forth in SEQ ID NO: 26 (Fragment 26).

TABLE 34

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 396 | CGCCCCAT | 397 | ACGATA | −21 | 1478 | 1499 |
| region | | AGTATATT | | TCAGCG | | | |
| | | GGACAT | | AAGGTG | | | |
| | | | | CT | | | |
| | 398 | TCCAGGTC | 399 | GCTGCA | −28 | 384 | 412 |
| | | GCCCCATA | | CTGGTA | | | |
| | | GTA | | TCCGAT | | | |
| | | | | TT | | | |
| | 400 | GGGGCTC | 401 | ACGATA | 1052 | 1478 | 426 |
| | | CTGATTCA | | TCAGCG | | | |
| | | TAACC | | AAGGTG | | | |
| | | | | CT | | | |
| 60 bp-200 bp | 402 | CAAGCCA | 403 | ATTAAG | 430 | 535 | 105 |
| | | AATGCTGA | | AACCGA | | | |
| | | CAAAA | | CGCCAG | | | |
| | | | | TG | | | |
| | 404 | CGGTTCTG | 405 | TGAATC | 397 | 496 | 99 |
| | | GCTCAGGT | | CCACAG | | | |
| | | AGTT | | CGTCAT | | | |
| | | | | TA | | | |
| | 406 | TTATTTAG | 407 | GCGAAG | 902 | 1007 | 105 |
| | | AGCCGCG | | ATCCTCT | | | |
| | | CTGAC | | GGTAACG | | | |
| | 408 | AGCATCCC | 409 | GCACCA | 732 | 819 | 87 |
| | | CCTTGTTA | | GCAGTA | | | |
| | | TTGA | | CATCGA | | | |
| | | | | GA | | | |
| | 410 | ATACCCGC | 411 | GCTACG | 1316 | 1421 | 105 |
| | | TTTCTCAA | | TGCTGG | | | |
| | | GTGC | | GGTATC | | | |
| | | | | TC | | | |
| 300 bp-500 bp | 412 | CGGGCCA | 413 | GTGTTC | 864 | 1265 | 401 |
| | | TACATCCA | | GGCTTG | | | |
| | | GTAAT | | CAGCTA | | | |
| | | | | TC | | | |
| | 414 | TTCTTTAG | 415 | CTATCG | 853 | 1250 | 397 |
| | | CTTCGGGC | | GGGGCG | | | |
| | | CATA | | TAGAGAA | | | |
| | 416 | TAATGACG | 417 | GATGTA | 477 | 876 | 399 |
| | | CTGTGGGA | | TGGCCC | | | |
| | | TTCA | | GAAGCT | | | |
| | | | | AA | | | |
| | 418 | GTCAAGC | 419 | GCACCA | 428 | 819 | 391 |
| | | CAAATGCT | | GCAGTA | | | |
| | | GACAA | | CATCGA | | | |
| | | | | GA | | | |

Table 35 lists additional primers contemplated by the present invention that may be used to identify the sequence as set forth in SEQ ID NO: 27 (Fragment 27).

TABLE 35

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 420 | CGGGCAA | 421 | CAGATC | −33 | 570 | 603 |
| region | | AGACTAC | | ATAGGT | | | |
| | | ACACAG | | GTAATG | | | |
| | | | | ATCGAA | | | |
| 60 bp-200 bp | 422 | GTATGCAG | 423 | AAACCG | 67 | 185 | 118 |
| | | GAAAGCA | | CTCAAA | | | |
| | | CCACA | | GGTGAA | | | |
| | | | | TG | | | |
| | 424 | ATACGTTT | 425 | CGGCAA | 186 | 302 | 116 |
| | | TCACGCCG | | TACGGG | | | |
| | | TTTC | | TCAGTA | | | |
| | | | | AG | | | |
| 300 bp-500 bp | 426 | ATCGTTTT | 427 | CGTAAA | 122 | 510 | 388 |
| | | GGCTTTGG | | TATCGG | | | |
| | | TGTC | | GAGGCG | | | |
| | | | | TA | | | |
| | 428 | TGGTATTG | 429 | CGTAAA | 8 | 510 | 502 |
| | | TGCGTACG | | TATCGG | | | |
| | | TGGT | | GAGGCG | | | |
| | | | | TA | | | |

Table 36 lists additional primers contemplated by the present invention that may be used to identify the sequence as set forth in SEQ ID NO: 28 (Fragment 28).

TABLE 36

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 430 | CCCTGGTG | 431 | AGCTCC | −37 | 760 | 797 |
| region | | CAGGACA | | CCATGG | | | |
| | | TAGAT | | TTTGCTAT | | | |
| | 432 | GACAACA | 433 | TTGCTAT | −49 | 747 | 796 |
| | | AACTCCCC | | CGGACA | | | |
| | | TGGTG | | TGGGTTA | | | |
| 60 bp-200 bp | 434 | CTGGTTCG | | GCACGT | 276 | 373 | 97 |
| | | TCCACTTT | | CGTTTG | | | |
| | | CGAT | | GAATTA | | | |
| | | | | GG | | | |
| | 436 | AGCTCTCC | 437 | AGCCCA | 135 | 242 | 107 |
| | | TGCCTGAA | | GACTGG | | | |
| | | CGTA | | CTACTG | | | |
| | | | | AA | | | |
| | 438 | CGACCCG | 439 | GCCGAG | 528 | 644 | 116 |
| | | AGTAAAA | | CAATAA | | | |
| | | GAACGA | | CACCAC | | | |
| | | | | TT | | | |
| 300 bp-500 bp | 440 | AGCCAGA | 441 | GCCGAG | 248 | 644 | 396 |
| | | TCCAGAA | | CAATAA | | | |
| | | GATTGC | | CACCAC | | | |
| | | | | TT | | | |
| | 442 | AGCTCTCC | 443 | GGTCGC | 135 | 532 | 397 |
| | | TGCCTGAA | | TAAGGT | | | |
| | | CGTA | | CATTGC | | | |
| | | | | TT | | | |
| | 444 | TCGATACG | 445 | TTTTACT | 39 | 541 | 502 |
| | | TAATGCGA | | CGGGTC | | | |
| | | AGCA | | GCTAAGG | | | |

Table 37 lists additional primers contemplated by the present invention that may be used to identify the sequence as set forth in SEQ ID NO: 29 (Fragment 29).

TABLE 37

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 446 | GGCGTGGT | 447 | GGGCGA | −39 | 284 | 323 |
| region | | ACATGGAT | | CGAATG | | | |
| | | ATGA | | TATTCA | | | |
| | | | | AA | | | |
| | 448 | CCAGGCG | 449 | GGGCGA | −42 | 284 | 326 |
| | | TGGTACAT | | CGAATG | | | |
| | | GGATA | | TATTCA | | | |
| | | | | AA | | | |
| 60 bp-200 bp | 450 | GCGGTGG | 451 | ATTGGT | 116 | 210 | 94 |
| | | CTACACTA | | ACCCAG | | | |
| | | TGGTT | | TTCGGT | | | |
| | | | | GA | | | |
| | 452 | ATGGCTTC | 453 | TGGTAC | 98 | 208 | 110 |
| | | ACGGTTAA | | CCAGTT | | | |
| | | ATGC | | CGGTGA | | | |
| | | | | TT | | | |
| | 454 | GGCGGAT | 455 | ACGCAG | 0 | 82 | 82 |
| | | CTGTATAC | | AAAAGG | | | |
| | | GCAAT | | CAGCTA | | | |
| | | | | AC | | | |
| 300 bp-500 bp | 456 | TGTATACG | 457 | TGGTAC | 8 | 208 | 200 |
| | | CAATCGG | | CCAGTT | | | |
| | | CTTTG | | CGGTGA | | | |
| | | | | TT | | | |

Table 38 lists additional primers contemplated by the present invention that may be used to identify the sequence as set forth in SEQ ID NO: 30 (Fragment 30).

TABLE 38

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 458 | TCATCGGA | 459 | AGCGGG | −27 | 935 | 962 |
| region | | TCTTTCCC | | TTTGAA | | | |
| | | TTGT | | AGGATG | | | |
| | | | | TA | | | |
| | 460 | AGTTGCCA | 461 | AAGCGG | 494 | 936 | 442 |
| | | ACCTTGAT | | GTTTGA | | | |
| | | CCTG | | AAGGAT | | | |
| | | | | GT | | | |
| | 462 | TCATCGGA | 463 | GAATCA | −27 | 421 | 448 |
| | | TCTTTCCC | | GGAACG | | | |
| | | TTGT | | GCTTTTTG | | | |
| 60 bp-200 bp | 464 | CAGTTGCC | 465 | CCTGCT | 493 | 591 | 98 |
| | | AACCTTGA | | GAAACA | | | |
| | | TCCT | | TGGCAA | | | |
| | | | | TA | | | |
| | 466 | ACCGAAA | 467 | CAATGC | 681 | 780 | 99 |
| | | AAGAGCA | | AAAATC | | | |
| | | AGAGCA | | CCATCC | | | |
| | | | | AT | | | |
| | 468 | ATTCGGAT | 469 | AGGATC | 418 | 512 | 94 |
| | | GGTATCGA | | AAGGTT | | | |
| | | CGAA | | GGCAAC | | | |
| | | | | TG | | | |
| 300 bp-500 bp | 470 | TGCCGGTA | 471 | CCCTGA | 211 | 604 | 393 |
| | | GTGTTATG | | AAAGAC | | | |
| | | GACA | | TCCTGCTG | | | |
| | 472 | AAGGTAA | 473 | AGGATC | 135 | 512 | 377 |
| | | ACTCTGCG | | AAGGTT | | | |
| | | CTCCA | | GGCAAC | | | |
| | | | | TG | | | |
| | 474 | TGCCGGTA | 475 | TCCCTG | 211 | 605 | 394 |
| | | GTGTTATG | | AAAAGA | | | |
| | | GACA | | CTCCTG | | | |
| | | | | CT | | | |
| | 476 | GCTGCCG | 477 | CCCTGA | 209 | 604 | 395 |
| | | GTAGTGTT | | AAAGAC | | | |
| | | ATGGA | | TCCTGCTG | | | |
| | 478 | AAACAAA | 479 | CAATGC | 389 | 780 | 391 |
| | | GCGTCGC | | AAAATC | | | |
| | | AAAAAG | | CCATCC | | | |
| | | | | AT | | | |

Table 39A lists additional primers contemplated by the present invention that may be used to identify the sequence as set forth in SEQ ID NO: 31 (Fragment 31).

TABLE 39A

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 480 | TACGGTGA | 481 | CTGCAA | −15 | 830 | 845 |
| region | | AAGAGTG | | CAATGG | | | |
| | | GCATT | | CTTTTTGT | | | |
| | 482 | TGTCTGCA | 483 | CTCGTTC | 436 | 838 | 402 |
| | | TAATTGGC | | CCTGCA | | | |
| | | TTTACC | | ACAATG | | | |
| | 484 | ACGGTGA | 485 | TGGGCT | −14 | 407 | 421 |
| | | AAGAGTG | | TTCTGTA | | | |
| | | GCATTG | | GGTTTT | | | |
| | | | | GA | | | |
| 60 bp-200 bp | 486 | GGCTACTT | 487 | GGGACG | 77 | 161 | 84 |
| | | CTCCCCAC | | TTACGC | | | |
| | | CATT | | AAATTT | | | |
| | | | | CT | | | |
| | 488 | TTCTCGTC | 489 | TGGGCT | 318 | 407 | 89 |
| | | AGGCATTT | | TTCTGTA | | | |
| | | TTCC | | GGTTTT | | | |
| | | | | GA | | | |
| | 490 | TGATCAAA | 491 | CTGCAA | 723 | 830 | 107 |
| | | CCAGCCAT | | CAATGG | | | |
| | | CAAC | | CTTTTTGT | | | |
| | 492 | TCGTCAGG | 493 | TGGGCT | 321 | 407 | 86 |
| | | CATTTTTC | | TTCTGTA | | | |
| | | CTTT | | GGTTTT | | | |
| | | | | GA | | | |
| 300 bp-500 bp | 494 | GGCTACTT | 495 | GGGCAA | 77 | 471 | 394 |
| | | CTCCCCAC | | CAGCCA | | | |
| | | CATT | | TAGGTA | | | |
| | | | | AA | | | |
| | 496 | TCTCGTCA | 497 | TCAGTT | 319 | 745 | 426 |
| | | GGCATTTT | | GATGGC | | | |
| | | TCCT | | TGGTTT | | | |
| | | | | GA | | | |

Table 39B lists additional primers contemplated by the present invention that may be used to identify the sequence as set forth in SEQ ID NO: 32 (Fragment 32).

TABLE 39B

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 498 | TGAAAAA | 499 | GCGAAT | 132 | 231 | 99 |
| region | | TAACTTAA | | TATTTA | | | |
| | | ATAAGGG | | GTACAA | | | |
| | | ATGG | | AAAGCG | | | |
| | | | | TA | | | |
| 60 bp-200 bp | 500 | TTTCATTA | 501 | AATATA | 110 | 171 | 61 |
| | | TGCTATTT | | AATATA | | | |
| | | AAGATGT | | TCCCAT | | | |
| | | GA | | CCCTTAT | | | |
| | | | | TT | | | |
| | 502 | CAGAGTA | 503 | CCCATC | 4 | 158 | 154 |
| | | AAAATGT | | CCTTATT | | | |
| | | AACGCTGA | | TAAGTT | | | |
| | | | | ATTTTTC | | | |

Table 40 lists additional primers contemplated by the present invention that may be used to identify the sequence as set forth in SEQ ID NO: 33 (Fragment 33).

TABLE 40

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 504 | GCAACAT | 505 | TGATTTT | −30 | 2460 | 2490 |
| region | | CAAAATG | | CGTCAG | | | |
| | | GCTGAG | | ACTGCA | | | |
| | | | | AG | | | |
| | 506 | GCAACAT | 507 | AACCTT | −30 | 252 | 282 |
| | | CAAAATG | | TCAACC | | | |
| | | GCTGAG | | GGAGTT | | | |
| | | | | CA | | | |
| | 508 | TGGCCTTG | 509 | TGATTTT | 1536 | 2460 | 924 |
| | | TACCAATT | | CGTCAG | | | |
| | | CCTT | | ACTGCA | | | |
| | | | | AG | | | |
| 60 bp-200 bp | 510 | TCCGATGA | 511 | CAATTG | 66 | 154 | 88 |
| | | AACATCA | | AAAACA | | | |
| | | CCATC | | TGGCCA | | | |
| | | | | GA | | | |
| | 512 | GATGTCCG | 513 | CAATTG | 62 | 154 | 92 |
| | | ATGAAAC | | AAAACA | | | |
| | | ATCACC | | TGGCCA | | | |
| | | | | GA | | | |
| | 514 | TGGCCTTG | 515 | TTTTTCA | 1536 | 1671 | 135 |
| | | TACCAATT | | GTAAGC | | | |
| | | CCTT | | TCAGAC | | | |
| | | | | AAATCA | | | |
| 300 bp-500 bp | 516 | TGGCCTTG | 517 | AAACAG | 1536 | 1940 | 404 |
| | | TACCAATT | | ATGTCC | | | |
| | | CCTT | | CGAAAA | | | |
| | | | | TCA | | | |

Table 41 lists additional primers contemplated by the present invention that may be used to identify the sequence as set forth in SEQ ID NO: 34 (Fragment 34).

TABLE 41

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 518 | CCTGCTGA | 519 | TAATCA | −49 | 1410 | 1459 |
| region | | TAGTGCCA | | GGAACA | | | |
| | | TGAA | | GCCGGA | | | |
| | | | | AG | | | |
| | 520 | CCTGCTGA | 521 | CGGTAA | −49 | 473 | 522 |
| | | TAGTGCCA | | GACACC | | | |
| | | TGAA | | AGCCTT | | | |
| | | | | GA | | | |
| 60 bp-200 bp | 522 | TCCTTGCC | 523 | GAGATC | 1335 | 1436 | 101 |
| | | TCATGTGT | | AAGCGT | | | |
| | | TCTG | | TTCCCA | | | |
| | | | | AG | | | |
| | 524 | GGAATTGC | 525 | AGCGAA | 496 | 592 | 96 |
| | | GAGTGAG | | CGAACA | | | |
| | | GTCTT | | GCTCAG | | | |
| | | | | AT | | | |
| | 526 | AGGTCTTA | 527 | TGTCGA | 509 | 603 | 94 |
| | | CCATTGGC | | GAATAA | | | |
| | | TGGA | | GCGAAC | | | |
| | | | | GA | | | |
| 300 bp-500 bp | 528 | CACTTTGC | 529 | AGCGAA | 190 | 592 | 402 |
| | | ATGAAAG | | CGAACA | | | |
| | | GGCTTA | | GCTCAG | | | |
| | | | | AT | | | |
| | 530 | AGGATGCT | 531 | TTGATA | 9 | 366 | 357 |
| | | GGATCAA | | GCTTAG | | | |
| | | AATGC | | CGCCCA | | | |
| | | | | AT | | | |
| | 532 | TCTCACCT | 533 | AGCGAA | 181 | 592 | 411 |
| | | TCACTTTG | | CGAACA | | | |
| | | CATGA | | GCTCAG | | | |
| | | | | AT | | | |

Table 42 lists additional primers contemplated by the present invention that may be used to identify the sequence as set forth in SEQ ID NO: 35 (Fragment 35).

TABLE 42

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 534 | TCACATGT | 535 | GCAACC | −14 | 1498 | 1512 |
| region | | GTTGCACC | | TTTTGCT | | | |
| | | TTTACA | | TTAATGt | | | |
| | | | | TTTT | | | |
| | 536 | TGCACAA | 537 | GCAACC | 1086 | 1498 | 412 |
| | | CTGGCCTA | | TTTTGCT | | | |
| | | TGTTT | | TTAATGt | | | |
| | | | | TTTT | | | |
| | 538 | GGAGAGT | 539 | AGTTCA | −49 | 351 | 400 |
| | | GCGGGGT | | TTCGCA | | | |
| | | ATTTTA | | ACCGTT | | | |
| | | | | TT | | | |
| 60 bp-200 bp | 540 | TGCTATTG | 541 | CCCGCT | 1245 | 1346 | 101 |
| | | GTGAATG | | TCATCT | | | |
| | | GCAAA | | GATGGT | | | |
| | | | | AT | | | |
| | 542 | GTTGGCAG | 543 | CTCCAC | 866 | 981 | 115 |
| | | GTTGCTCA | | CATAAT | | | |
| | | ATTC | | TCCGCTTG | | | |
| | 544 | TGAGCAA | 545 | CGCAAC | 239 | 343 | 104 |
| | | GAGCATA | | CGTTTTG | | | |
| | | GGTTTTGA | | TTTTCtT | | | |
| 300 bp-500 bp | 546 | GTTGGCAG | 547 | TGCCAT | 866 | 1262 | 396 |
| | | GTTGCTCA | | TCACCA | | | |
| | | ATTC | | ATAGCA | | | |
| | | | | AA | | | |
| | 548 | GCGGAATT | 549 | ATAGAC | 965 | 1364 | 399 |
| | | ATGGTGG | | GTCGCC | | | |
| | | AGAAA | | AGATTT | | | |
| | | | | CC | | | |
| | 550 | TACAAAG | 551 | TGCCAT | 856 | 1262 | 406 |
| | | GCTGTTGG | | TCACCA | | | |
| | | CAGGT | | ATAGCA | | | |
| | | | | AA | | | |
| | 552 | GGATGAT | 553 | CACTTC | 505 | 892 | 387 |
| | | GAATCAAT | | CGAATT | | | |
| | | GCCAAA | | GAGCAA | | | |
| | | | | CC | | | |

Table 43 lists additional primers contemplated by the present invention that may be used to identify the sequence as set forth in SEQ ID NO: 36 (Fragment 36).

TABLE 43

| | | | | | | |
|---|---|---|---|---|---|---|
| SEQ | | SEQ | | | | pcr |
| ID | | ID | | | | product |
| NO: | 5′ primer | NO: | 3′ primer | coordinates | coordinates | length |

| | | | | | | | |
|---|---|---|---|---|---|---|---|
| flanking | 554 | CGAGCAC | 555 | GAGTGG | −47 | 758 | 805 |
| region | | TGTTATAG | | TCGGGG | | | |
| | | TAATTTCA | | TATTGT | | | |
| | | GAAG | | GT | | | |
| | 556 | TGAAGTTT | 557 | GATGAG | 371 | 761 | 390 |
| | | GTGCCAC | | TGGTCG | | | |
| | | GGTAA | | GGGTAT | | | |
| | | | | TG | | | |
| | 558 | CGAGCAC | 559 | CGACCT | −47 | 345 | 392 |
| | | TGTTATAG | | GAAAAG | | | |
| | | TAATTTCA | | CCCAAA | | | |
| | | GAAG | | TA | | | |
| 60 bp-200 bp | 560 | ACAGTCG | 561 | CGACCT | 235 | 345 | 110 |
| | | AGCCAGC | | GAAAAG | | | |
| | | TTCAAT | | CCCAAA | | | |
| | | | | TA | | | |
| | 562 | ACAGTCG | 563 | CCCAAA | 235 | 333 | 98 |
| | | AGCCAGC | | TACTTC | | | |
| | | TTCAAT | | GGGAGC | | | |
| | | | | TA | | | |
| | 564 | CCACCATC | 565 | TGACAT | 415 | 523 | 108 |
| | | ACCCTCAA | | GGTTGA | | | |
| | | GTTC | | CAACAG | | | |
| | | | | CA | | | |
| | 566 | CCCGAAG | 567 | GAGGGT | 319 | 428 | 109 |
| | | TATTTGGG | | GATGGT | | | |
| | | CTTTT | | GGTGAT | | | |
| | | | | GT | | | |
| 300 bp-500 bp | 568 | TTGAATCA | 569 | TGAACT | 33 | 435 | 402 |
| | | AAAATGC | | TGAGGG | | | |
| | | ACGACA | | TGATGG | | | |
| | | | | TG | | | |
| | 570 | CCCGAAG | 571 | CAAATA | 319 | 715 | 396 |
| | | TATTTGGG | | GTCCCC | | | |
| | | CTTTT | | GCCCTTTA | | | |
| | 572 | CCGAAAA | 573 | TGACAT | 104 | 523 | 419 |
| | | GAGGAGT | | GGTTGA | | | |
| | | TGAACG | | CAACAG | | | |
| | | | | CA | | | |

Another method based on single nucleotide primer (SNuPe) extension may also be used to detect the M17 sequences of the present invention.

A number of primer extension-based characterizations of bacteria have been reported, including the phylotyping of Listeria monocytogenes [Rudi et al, 2003, FEMS Microbiol. Lett. 220, 9-14; Ducey et al, 2007, Microbiol. 73, 133-147] and Escherichia coli strains [Hommais et al, 2005, Appl. Environ. Microbiol. 71, 4784-4792] and the rapid identification of Brucella isolates [Scott et al, 2007, Appl. Environ. Microbiol. 73, 7331-7337]. These studies demonstrated the good discrimination potential and high taxonomical resolution of SNuPe analyses with primer extension.

The principle of SNuPE is described herein below and in Nikolausz et al [Biochemical Society Transactions (2009) Volume 37, part 2], incorporated herein by reference. The method benefits from the high fidelity of DNA polymerases while incorporating nucleotides or nucleotide analogues, resulting in a highly specific distinction of sequence variants. When a specific primer hybridizes upstream from the target nucleotide position, a DNA polymerase incorporates a labelled nucleoside triphosphate, which terminates the reaction and results in a labelled extended primer.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Sequencing of M17

Materials and Methods

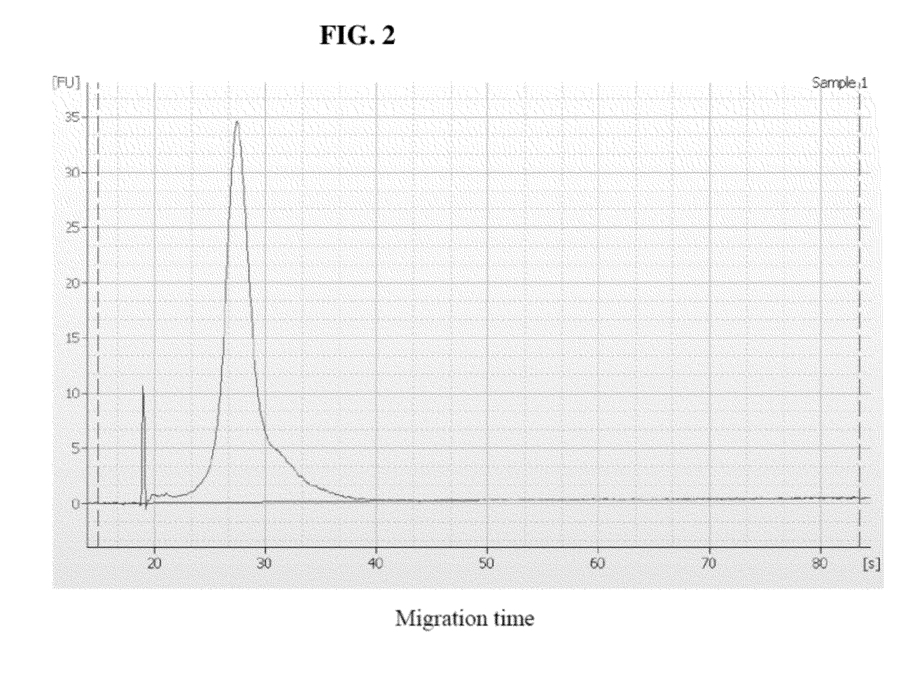

454 Genomic Sequencing of M17p: M17p gDNA was fragmented and an 8 kb paired-end library suitable for 454 platform sequencing was prepared (following manufacturer's instructions. QC analysis of the generated paired-end library using an Agilent 2100 Bioanalyzer (FIG. 2) indicated that the library was of acceptable quality, containing the expected fragment size and yield, for continued sample processing.

The paired-end library was used in emulsion PCR (GS Titanium LV emPCR Kit (Lib-L), Roche) following manufacturers instructions. The generated DNA beads passed QC analysis with an enrichment of 20.3. These DNA beads were used in ½ Titanium plate 454 sequencing (following manufacturers instructions), using the GS Titanium Sequencing Kit XLR70.

The raw 454 sequence data was then assembled with the Roche Newbler (2.0.00.22) software.

Unique fragment identification: Sequence data available from public sources was compared to the M17p 454 sequence data in the Cross-Match software package (using the default screening settings) and 36 fragments were identified that are unique to the M17p strain.

Results

The sequencing results summary is provided in Table 44, herein below.

TABLE 44

| | | Average Read | |
|---|---|---|---|
| | Read Number | Length | |
| Sample Name | (No. of bases) | (No. of bases) | Total Bases |
| M17p | 529,038 | 378 | 200,066,401 |

The assembly results are summarized in Table 45, herein below.

TABLE 45

| | | | Largest | Number of | Largest |
|---|---|---|---|---|---|
| Sample | Number of | Number of | Scaffold | Large | Contig |
| Name | Scaffolds* | Bases | Size | Contigs | Size |
| M17p | 5 | 5,010,882 | 5,005,431 | 78 | 440,798 |

*A Scaffold is a collection of sequence contigs that have been oriented and spaced with respect to one another based on paired-end sequencing data. Scaffold sequences are represented in FASTA format and contain stretches of N's to represent the gaps between contigs.

The sequences were then analyzed using the Blastn algorithm (the NCBI website) to search the nr Database collection: All GenBank+EMBL+DDBJ+PDB sequences under default settings.

The sequence of the 36 fragments identified as being unique according to the Cross-Match software package and Blast analysis may be identified as set forth in Table 46, herein below.

TABLE 46

| frag. | SEQ ID NO: | Contig | start | end | length |
|---|---|---|---|---|---|
| 1 | 1 | contig00078 | 62663 | 63843 | 1181 |
| 2 | 2 | contig00078 | 66656 | 66825 | 170 |
| 3 | 3 | contig00084 | 21765 | 23445 | 1681 |
| 4 | 4 | contig00095 | 18923 | 19111 | 189 |
| 5 | 5 | contig00097 | 11127 | 11542 | 416 |
| 6 | 6 | contig00100 | 28867 | 29421 | 555 |
| 7 | 7 | contig00100 | 29604 | 30960 | 1357 |
| 8 | 8 | contig00100 | 31120 | 31914 | 795 |
| 9 | 9 | contig00100 | 32458 | 32794 | 337 |
| 10 | 10 | contig00100 | 63457 | 63982 | 526 |
| 11 | 11 | contig00100 | 64229 | 64692 | 464 |
| 12 | 12 | contig00101 | 13634 | 14112 | 479 |
| 13 | 13 | contig00101 | 14323 | 14718 | 396 |
| 14 | 14 | contig00112 | 412197 | 412869 | 673 |
| 15 | 15 | contig00112 | 419061 | 419866 | 806 |
| 16 | 16 | contig00112 | 428236 | 428583 | 348 |
| 17 | 17 | contig00112 | 428632 | 428917 | 286 |
| 18 | 18 | contig00112 | 429912 | 430417 | 506 |
| 19 | 19 | contig00112 | 430856 | 431018 | 163 |
| 20 | 20 | contig00112 | 431234 | 431635 | 402 |
| 21 | 21 | contig00112 | 431796 | 432887 | 1092 |
| 22 | 22 | contig00112 | 434683 | 435243 | 561 |
| 23 | 23 | contig00113 | 240 | 518 | 279 |
| 24 | 24 | contig00113 | 15607 | 16798 | 1192 |
| 25 | 25 | contig00115 | 32035 | 32811 | 777 |
| 26 | 26 | contig00115 | 35651 | 37087 | 1437 |
| 27 | 27 | contig00115 | 40119 | 40664 | 546 |
| 28 | 28 | contig00115 | 40865 | 41583 | 719 |
| 29 | 29 | contig00115 | 61928 | 62161 | 234 |
| 30 | 30 | contig00115 | 62302 | 63238 | 937 |
| 31 | 31 | contig00116 | 630 | 1517 | 888 |
| 32 | 32 | contig00117 | 317892 | 318076 | 185 |
| 33 | 33 | contig00122 | 68537 | 70960 | 2424 |
| 34 | 34 | contig00122 | 72789 | 74195 | 1407 |
| 35 | 35 | contig00122 | 76097 | 77545 | 1449 |
| 36 | 36 | contig00122 | 80315 | 81044 | 730 |

A summary of the ten longest unique identified fragments is shown in Table 47.

TABLE 47

| Fragment | Fragment length |
|---|---|
| frag-33.fasta | 2,424 |
| frag-3.fasta | 1,681 |
| frag-35.fasta | 1,449 |
| frag-26.fasta | 1,437 |
| frag-34.fasta | 1,407 |
| frag-7.fasta | 1,357 |
| frag-24.fasta* | 1,192 |
| frag-1.fasta | 1,181 |
| frag-21.fasta | 1,092 |
| frag-30.fasta | 937 |

### Example 2

### PCR Based Unique Fragments Confirmation Assay

Materials and Methods

Samples: A total of 74 samples were processed during this project:
72 E. coli samples from the ECOR culture collection.
E. coli M17p (M17 parent) Deposit No. ATCC Deposit No. 202226 (DSM 12799).
E. coli M17SNAR (nalidixic acid-resistant strain) Deposit No. 7295.

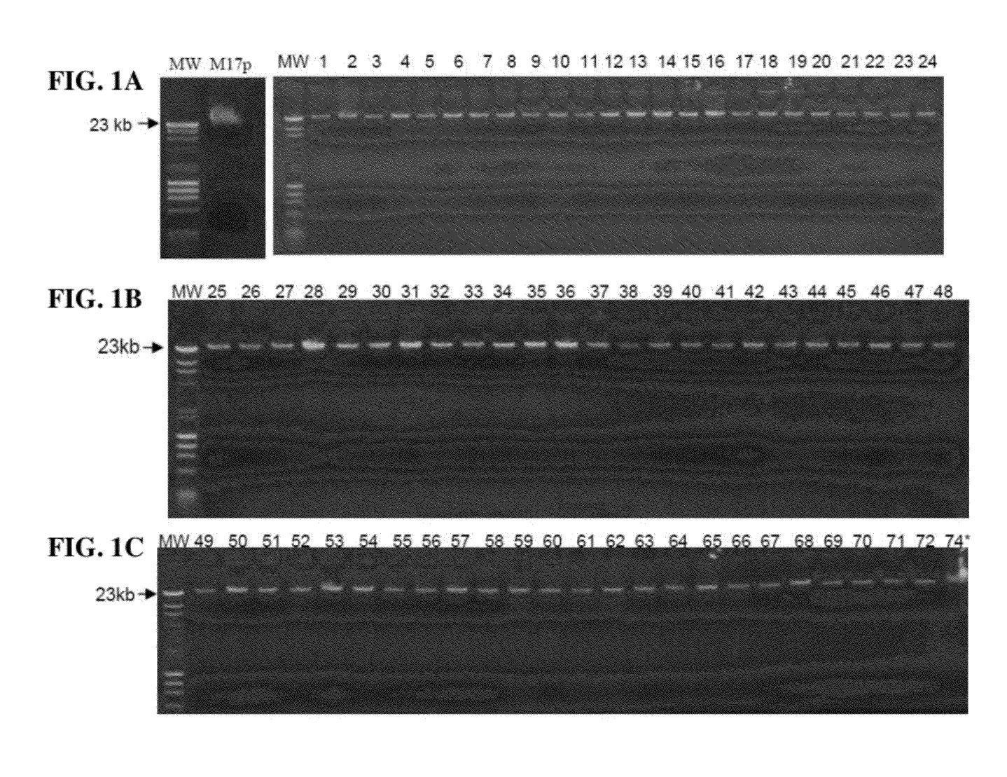

DNA Purification: SeqWright extracted the E. coli gDNA from M17p, M17SNAR and the 72 ECOR collection E. coli culture samples with the Promega Wizard™ Genomic DNA Purification Kit (following manufacturer's instructions). A quality control (QC) inspection and rough quantitation of the extracted gDNA samples was performed by agarose gel electrophoresis and UV-induced ethidium bromide fluorescence (FIG. 1). Sample quality was compared visually on the gel against a λ DNA-Hind III Digest and ΦX-174-RF DNA, Hae III digest molecular weight (MW) size marker. All 74 E. coli gDNA samples were of acceptable quality for continued processing. Sample quality was considered to be acceptable if the extracted gDNA supplied a single visible band while lacking any significant degradation products (degraded DNA seen as smear of small fragments).

PCR Based Unique Fragments Confirmation Assay: The 10 unique fragments highlighted in Table 7 were selected for the development of a PCR based assay with an amplicon size range of 400-550 bp. Primers were designed for the assay using Primer3 software from MIT. Fragments 24 and 35 from Table 7 were not further processed because higher quality amplicons were obtained from the other eight unique fragments. Additionally, the larger size of the unique regions of Fragments 33 and 34 allowed for two non-overlapping amplicons to be designed for each fragment. For the PCR assay, a total of 10 Primer3 designed primer pairs were selected (Table 48, herein below). Detailed information on the selected primers can be found in Table 49, herein below.

TABLE 48

| Unique | Primer | | | |
|---|---|---|---|---|
| Fragment | Name | Sequence | SEQ ID NO: | Amplicon |
| frag 30 | CP1 | AAGGTAAACT | 52 | 470 bp |
| | | CTGCGCTCCA | | |
| | CP2 | CCCTGAAAAG | 53 | |
| | | ACTCCTGCTG | | |
| frag 7 | CP3 | TGGTGCTAGAC | 54 | 516 bp |
| | | TCTGGGCTT | | |
| | CP4 | TGACGGAAAT | 55 | |
| | | ATCCACAGCA | | |
| frag 34 | CP5 | CGCTGTGGAA | 56 | 536 bp |
| | | AGTGACAGAA | | |
| | CP6 | AATGAATGAG | 57 | |
| | | CAAACCGAGG | | |
| frag 3 | CP7 | GCGATAACTG | 58 | 489 bp |
| | | GGCAAATGAT | | |
| | CP8 | ATTGCATGAG | 59 | |
| | | TCCAACCACA | | |
| frag 26 | CP9 | AAATCGGATA | 60 | 455 bp |
| | | CCAGTGCAGC | | |
| | CP10 | GCACCAGCAG | 61 | |
| | | TACATCGAGA | | |
| frag 33 | CP11 | TGCGAATCGAT | 37 | 516 bp |
| | | GATCTCAAG | | |
| | CP12 | TTGGTACAAG | 38 | |
| | | GCCATGTTGA | | |
| frag 33 | CP13 | GCTGTTTCATG | 39 | 545 bp |
| | | AACTCCGGT | | |
| | CP14 | TGGGGACGAA | 40 | |
| | | ATATCACCAT | | |
| frag 1 | CP15 | TCCGGGTTGAT | 41 | 497 bp |
| | | AACCATCAT | | |
| | CP16 | ACCACTCTGG | 42 | |
| | | TCCTTCATGC | | |
| frag 21 | CP17 | CCGTGGACGA | 43 | 451 bp |
| | | ATAGAGCATT | | |
| | CP18 | TTTTTGGTGCA | 44 | |
| | | TTGATTCCA | | |
| frag 34 | CP19 | ATCTGAGCTG | 45 | 451 bp |
| | | TTCGTTCGCT | | |
| | CP20 | TACCGGGAAA | 46 | |
| | | AATGGTCAAA | | |

TABLE 49

| | | Left | Right | | |
|---|---|---|---|---|---|
| | Pair score | primer | primer | Left | Right |
| Fragment of | (lower is | location, | location, | primer | primer |
| origin | better) | length | length | Tm | Tm |
| frag-30.fasta | 0.033 | 134, 20 | 603, 20 | 60.015 | 59.982 |
| frag-7.fasta | 0.0851 | 426, 20 | 941, 20 | 60.012 | 60.073 |
| frag-34.fasta | 0.0983 | 36, 20 | 571, 20 | 60.025 | 60.074 |
| frag-3.fasta | 0.1037 | 816, 20 | 1304, 20 | 59.929 | 59.967 |
| frag-26.fasta | 0.1191 | 364, 20 | 818, 20 | 60.103 | 60.016 |
| frag-33.fasta | 0.1282 | 1033, 20 | 1548, 20 | 59.907 | 59.964 |
| frag-33.fasta | 0.1324 | 223, 20 | 767, 20 | 60.119 | 60.014 |
| frag-1.fasta | 0.1334 | 206, 20 | 702, 20 | 60.014 | 60.12 |
| frag-21.fasta | 0.1886 | 177, 20 | 627, 20 | 60.096 | 59.907 |
| frag-34.fasta | 0.3238 | 572, 20 | 1022, 20 | 60.164 | 60.16 |

The PCR reactions in 25 ul contained 12.5 ul of AmpliTaq Gold 2× Master Mix, 0.2-1.0 uM of primers, and 50 ng of template DNA (with H₂O as negative control), and were performed for 30 cycles consisting of the following steps: denaturation at 95° C. for 30 s; annealing at 50° C. for 30 s; and extension at 72° C. for 60 s. The generated PCR products were checked on agarose gel.

Results

Three primer pairs (CP11 and CP12, CP13 and CP14, and CP19 and CP20) generated PCR products (single band on the gel) for M17p and M17SNAR but not for the 72 ECOR collection E. coli culture samples as shown in FIGS. 3-5, with summary for all 10 primer pairs provided in Table 50.

TABLE 50

| | Unique Fragment | | | | | | | | | |
|---|---|---|---|---|---|---|---|---|---|---|
| | 30 | 7 | 34 | 3 | 26 | 33 | 33 | 1 | 21 | 34 |
| | Primer Pair | | | | | | | | | |
| | CP1 + | CP3 + | CP5 + | CP7 + | CP9 + | CP11 + | CP13 + | CP15 + | CP17 + | CP19 + |
| | CP2 | CP4 | CP6 | CP8 | CP10 | CP12 | CP14 | CP16 | CP18 | CP20 |
| H₂O (neg. control) | − | − | − | − | − | − | − | − | − | − |
| M17p | + | + | + | + | + | + | + | + | + | + |
| M17 SNAR | + | + | + | + | + | + | + | + | + | + |
| ECOR-1 | − | − | − | − | − | − | − | + | − | − |
| ECOR-2 | + | − | − | − | − | − | − | + | − | − |
| ECOR-3 | − | − | − | − | − | − | − | + | − | − |
| ECOR-4 | + | − | − | − | + | − | − | + | − | − |
| ECOR-5 | − | − | − | − | + | − | − | + | − | − |
| ECOR-6 | + | − | − | − | + | − | − | + | − | − |
| ECOR-7 | − | − | − | − | + | − | − | + | − | − |
| ECOR-8 | − | + | + | − | − | − | − | + | − | − |
| ECOR-9 | − | − | − | − | + | − | − | + | − | − |
| ECOR-10 | + | + | − | − | + | − | − | + | − | − |
| ECOR-11 | + | − | − | − | + | − | − | + | − | − |
| ECOR-12 | + | − | − | − | + | − | − | + | − | − |
| ECOR-13 | + | − | − | − | + | − | − | + | − | − |
| ECOR-14 | + | − | − | − | + | − | − | + | − | − |
| ECOR-15 | − | − | − | − | + | − | − | + | − | − |
| ECOR-16 | − | − | − | − | + | − | − | + | − | − |
| ECOR-17 | − | + | − | − | + | − | − | + | − | − |
| ECOR-18 | + | − | − | − | + | − | − | + | − | − |
| ECOR-19 | + | − | − | − | + | − | − | + | − | − |
| ECOR-20 | + | − | − | − | + | − | − | + | − | − |
| ECOR-21 | − | − | − | − | + | − | − | + | − | − |
| ECOR-22 | − | − | − | − | + | − | − | + | − | − |
| ECOR-23 | − | − | − | − | + | − | − | + | − | − |
| ECOR-24 | − | − | + | − | + | − | − | + | − | − |
| ECOR-25 | − | − | − | − | − | − | − | + | − | − |
| ECOR-26 | − | − | − | − | − | − | − | + | − | − |
| ECOR-27 | − | − | − | − | + | − | − | + | − | − |
| ECOR-28 | − | − | − | − | − | − | − | + | − | − |
| ECOR-29 | − | − | + | − | − | − | − | − | − | − |
| ECOR-30 | − | − | − | − | − | − | − | − | − | − |
| ECOR-31 | − | − | + | − | + | − | − | − | − | − |
| ECOR-32 | − | − | − | − | − | − | − | − | − | − |
| ECOR-33 | + | − | − | − | + | − | − | − | − | − |
| ECOR-34 | + | − | − | − | + | − | − | − | − | − |
| ECOR-35 | − | − | − | − | + | − | − | + | − | − |
| ECOR-36 | − | − | − | − | + | − | − | − | − | − |
| ECOR-37 | + | − | − | − | + | − | − | − | − | − |
| ECOR-38 | − | + | − | − | + | − | − | − | − | − |
| ECOR-39 | − | + | − | − | + | − | − | − | − | − |
| ECOR-40 | + | + | − | − | + | − | − | − | − | − |
| ECOR-41 | + | + | − | − | − | − | − | − | − | − |
| ECOR-42 | + | − | − | − | + | − | − | − | − | − |
| ECOR-43 | + | + | + | − | + | − | − | − | − | − |
| ECOR-44 | − | − | + | − | − | − | − | + | − | − |
| ECOR-45 | − | − | − | − | − | − | − | + | − | − |
| ECOR-46 | − | − | − | − | + | − | − | + | − | − |
| ECOR-47 | − | + | − | − | + | − | − | + | − | − |
| ECOR-48 | − | − | − | − | + | − | − | − | − | − |
| ECOR-49 | − | + | − | − | + | − | − | + | − | − |
| ECOR-50 | − | + | − | − | + | − | − | + | − | − |
| ECOR-51 | − | + | + | − | − | − | − | − | − | − |
| ECOR-52 | − | + | + | − | − | − | − | − | − | − |
| ECOR-53 | − | + | − | − | − | − | − | − | + | − |
| ECOR-54 | − | + | + | − | − | − | − | − | − | − |
| ECOR-55 | − | + | + | − | − | − | − | − | − | − |
| ECOR-56 | − | + | + | − | − | − | − | − | − | − |
| ECOR-57 | − | + | + | − | + | − | − | − | − | − |
| ECOR-58 | − | − | − | − | + | − | − | − | − | − |
| ECOR-59 | − | + | + | − | + | − | − | − | − | − |
| ECOR-60 | − | + | + | − | + | − | − | − | − | − |
| ECOR-61 | − | + | + | − | + | − | − | − | − | − |
| ECOR-62 | − | + | + | − | − | − | − | − | − | − |
| ECOR-63 | − | + | + | − | − | − | − | − | − | − |
| ECOR-64 | − | + | + | − | − | − | − | − | − | − |
| ECOR-65 | − | − | + | + | − | − | − | − | + | − |
| ECOR-66 | − | + | + | − | − | − | − | − | − | − |
| ECOR-67 | − | − | + | − | − | − | − | − | − | − |
| ECOR-68 | − | − | + | − | − | − | − | − | − | − |
| ECOR-69 | − | − | + | − | − | − | − | − | − | − |
| ECOR-70 | − | − | − | − | − | − | − | + | − | − |
| ECOR-71 | − | − | − | − | + | − | − | + | − | − |
| ECOR-72 | − | − | − | − | − | − | − | + | − | − |

### Example 3

### Detection of E. coli M17p Strain Spiked in Biological Stool Samples

The following experiments were performed to test if the three unique M17 detection PCR assays identified as shown in Example 2 would work with DNA extracted from M17p cell spiked biological stool samples.

Materials and Methods

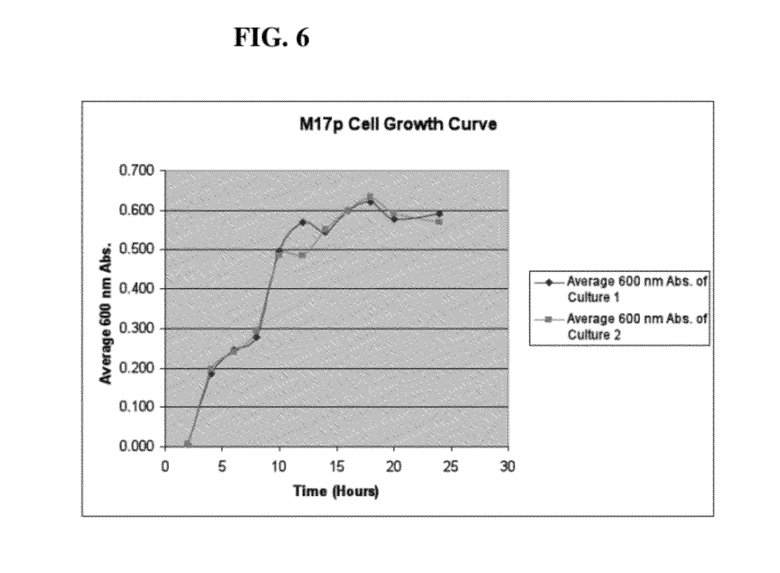

The E. coli M17p Strain Growth Curve (FIG. 6) was determined from the average 600 nm absorbance readings measured by Nanodrop ND-1000 Spectrophotometer over a time course of 2-24 hours as described below. A single colony of the E. coli M17p Strain was used to inoculate 5 mL of LB growth media in duplicate (Culture 1 and Culture 2) and the cell cultures were grown at 37° C. and 250 rpm. A sample of 100 μL cell culture was obtained from each of Culture 1 and Culture 2 at time points 2, 4, 6, 8, 10, 12, 14, 16, 18, 20, and 24 hours. Each sample was used to measure the 600 nm absorbance readings three times on a Nonodrop ND-1000 Spectrophotometer and the average reading for each sample at each time point was used to create the growth curve.

The growth curve (see FIG. 6) indicated that the 600 nm absorbance reading 0.5 by Nanodrop ND-1000 Spectrophotometer was approaching to the end of the logarithmic growth phase of the M17p cell cultures. (It should be appreciated that the OD600 reading measured by Nanodrop Spectrophotometer is approximately 10 fold less than other conventional spectrophotometers due to the use of shorter path length (1 mm vs. 10 mm) by Nanodrop Spectrophotometer. Therefore, e.g., the cell density from an OD600 absorbance reading 0.2 by Nanodrop is approximately equivalent to the cell density from an OD600 absorbance reading 2.0 by other conventional spectrophotometers.

DNA Extraction from M17p Cell Spiked Stool Samples: Serial cell dilutions were made by picking a single colony from an agar plate and mixing it well with 5 mL LB broth (reference as undiluted or 100). From this initial dilution, subsequent 10-1, 10-2, and 10-3 dilutions were made. One hundred microliters of each dilution was used to inoculate a 5 mL-LB culture. The cultures were grown at 37° C. and 250 rpm for 14 hours. The 600 nm Absorbance of the four 14 hour old cell cultures were measured three times for each by Nanodrop ND-1000 Spectrophotometer and the average 600 nm absorbance for each culture is listed in Table 51. The M17p cell culture from 10-1 inoculation listed in Table 51 was used for stool spiking experiment.

TABLE 51

| Cell culture | Average 600 nm Abs. |
|---|---|
| M17p Cell Culture from 10⁰ inoculation | 0.24 |
| M17p Cell Culture from 10⁻¹ inoculation | 0.21 |
| M17p Cell Culture from 10⁻² inoculation | 0.19 |
| M17p Cell Culture from 10⁻³ inoculation | 0.15 |

The M17p Cell Culture from 10⁻¹ inoculation with an average 600 nm absorbance reading 0.21 was selected for spiking experiment since it was well within the logarithmic growth phase. A serial dilution was made from this culture: undiluted, 10⁻¹, 10⁻², 10⁻³, 10⁴, 10⁻⁵, 10⁻⁶, 10⁻⁷, 10⁻⁸, 10⁻⁹, 10⁻¹⁰, 10⁻¹¹, 10⁻¹², and 10⁻¹³.

Aliquots of 180 mg biological stool samples were made in 2-mL tubes and spiked with 50 μL of cell dilutions listed above respectively. The spiked stool samples were mixed thoroughly.

DNAs were extracted from the spiked stool samples using QIAamp Stool DNA mini kit, including a no-spike stool sample control, a spike-buffer control (180 μL PBS buffer+50 μL of 10⁻³ cell dilution), and 10¹⁰ spike control (500 μL undiluted culture spun down and resuspended in 50 μL for spiking). 50 μL of the cell dilutions from the 10⁻⁶, 10⁻⁷, 10⁻⁸, and 10⁻⁹ serial dilutions (the same dilutions that were used to spike the stool samples) were plated on LB Agar plates in duplicate and grown overnight at 37° C. to determine the colony formation units per mL (CFU/mL). The colony counts from each dilution are listed in Table 52.

TABLE 52

| Dilutions | 10⁻⁶ | 10⁻⁷ | 10⁻⁸ | 10⁻⁹ |
|---|---|---|---|---|
| # of Colonies | 65 | 11 | 0 | 0 |
| from Plate A | | | | |
| # of Colonies | 56 | 6 | 0 | 0 |
| from Plate B | | | | |

Average from the 10⁻⁶ culture is ˜60 CFU/50 μL, which is converted to 1.2×103 CFU/mL. The undiluted culture that was used to spike the stool samples is estimated to have a 1.2×109 CFU/mL. The CFU/mL was estimated to be 1.2×108 CFU/mL for the 10⁻¹ dilution, 1.2×107 CFU/mL for the 10⁻² dilution, 1.2×106 CFU/mL for the 10⁻³ dilution, 1.2×105 CFU/mL for the 10⁻⁴ dilution, which converted to 3.3×108 CFU/gram spiked stool sample for the undiluted culture (100), 3.3×107 CFU/gram spiked stool sample for the 10⁻¹ dilution, 3.3×106 CFU/gram spiked stool sample for the 10⁻² dilution, 3.3×105 CFU/gram spiked stool sample for the 10⁻³ dilution, and 3.3×104 CFU/gram spiked stool sample for the 10⁻⁴ dilution.

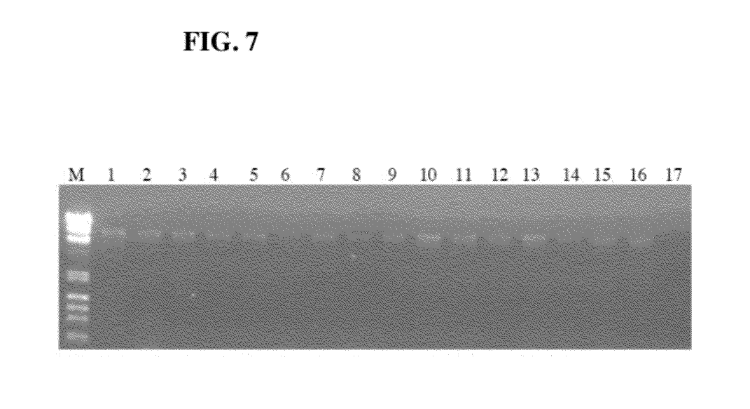

Two microliters of each of the DNAs extracted from the spiked stool samples, the no-spike stool sample and the spiked PBS buffer sample were analyzed on an agarose gel (see FIG. 7) and these DNAs were used for PCR amplification for M17p detection.

The total DNA extracted from the spiked stool samples and controls were used as templates in PCR reactions using the three M17 strain detection PCR assays developed in Example 2.

The primer sets for each of the three assays are CP11 and CP12, CP13 and CP14, and CP19 and CP20, details of which are provided in Tables 8 and 9, herein above.

All PCR reactions were set up as shown below with a total reaction volume of 20 μL:

| | |
|---|---|
| 1 μL DNA was assembled with 2X | 10 μL |
| Amplitaq Gold PCR Master Mix | |
| 10 X BSA | 2 μL |
| Primer 1 at 20 μM | 1 μL |
| Primer 2 at 20 μM | 1 μL |
| H₂0 | 5 μL |

PCR reactions include a no-template negative control (H20) and positive control (1 μL of undiluted cell culture (used to spike the stool samples) mixed with 50 μL H20 and incubate at 98° C. for 6 minutes. 1 μL of the cracked cell sample was used as the positive control in PCR).

Cycling Conditions:

95° C. for 5 minutes followed by 30 cycles of 95° C. for 30 seconds, 50° C. for 30 seconds, and 72° C. 1 minute. Then hold at 72° C. for 7 minutes and stopped by holding at 4° C. After PCR, 5 μL of each PCR product, resulting from the DNA extracted from the M17p spiked stool samples, was analyzed on an agarose gel.

Results

The results are presented in FIGS. 8-13.

PCR amplicons of expected size were detected in all three sets of primers used, in two replicate PCR assays on DNAs extracted from the 3.3×10⁸ CFU/gram spiked stool sample (or the undiluted culture (10°) spike), the 3.3×10⁷ CFU/gram spiked stool sample (or the 10⁻¹ dilution spike), and the 3.3×10⁶ CFU/gram spiked stool sample (or the 10⁻² dilution spike). There was no amplification observed in the negative control (no-template control). The positive control generated an amplicon of expected size. There was no amplification observed in the no-spike stool DNA control. The PBS buffer control spiked with a 10⁻³ dilution, generated an amplicon with similar intensity as the amplicon generated from 10⁻³ spiked stool DNA sample, indicating that PCR reactions are not inhibited from the stool DNA samples.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method of identifying an M17 strain of E. coli in a human sample, the method comprising analyzing DNA extracted from the human sample for a presence or absence of at least one M17 specific nucleic acid sequence of an M17 nucleic acid sequence being selected from the group consisting of SEQ ID NOs: 33, 34, 31, 3, 30, 35 and 36 under experimental conditions, said at least one M17 specific nucleic acid sequence being distinguishable from non M17 nucleic acid sequences in said DNA under said experimental conditions, wherein a presence of said at least one M17 specific nucleic acid sequence is indicative of M17 in the human sample.

2. The method of claim 1, wherein said M17 nucleic acid sequence is selected from the group consisting of SEQ ID NOs: 34 and 35.

3. The method of claim 1, wherein said M17 nucleic acid sequence is selected from the group consisting of SEQ ID NOs: 3, 30 and 36.

4. A method of identifying an M17 strain of E. coli in a human biological sample, the method comprising analyzing products of an amplification reaction using DNA extracted from the human biological sample and a primer pair which amplifies an M17 specific nucleic acid sequence of an M17 nucleic acid sequence, wherein said primer pair is selected from the group consisting of SEQ ID NOs: 37 and 38; SEQ ID NO: 39 and 40; and SEQ ID NOs: 45 and 46, wherein a product of said amplification reaction is indicative of an M17 strain of E. coli.

5. A method of identifying an M17 strain of E. coli in a human fecal sample, the method comprising analyzing DNA extracted from the human fecal sample for a presence or absence of at least one M17 specific nucleic acid sequence of an M17 nucleic acid sequence being selected from the group consisting of SEQ ID NOs: 1-36 under experimental conditions, said at least one M17 specific nucleic acid sequence being distinguishable from non M17 nucleic acid sequences in said DNA under said experimental conditions, wherein a presence of said at least one M17 specific nucleic acid sequence is indicative of M17 in the human fecal sample.

6. The method of claim 1, further comprising quantifying an amount of M17 in the sample.

7. The method of claim 1, wherein said analyzing is effected using at least one oligonucleotide being at least 13 bases which hybridizes to said M17 specific nucleic acid sequence to provide a detectable signal under said experimental conditions and which does not hybridize to said non M17 nucleic acid sequences to provide a detectable signal under said experimental conditions.

8. The method of claim 1, wherein said biological sample comprises a fecal sample.

9. The method of claim 1, wherein said analyzing is effected using two oligonucleotides, each of said two oligonucleotides being at least 13 bases.

10. A primer pair which amplifies an M17 specific nucleic acid sequence of an M17 nucleic acid sequence being selected from the group consisting of SEQ ID NOs: 1-36 under experimental conditions and does not amplify a non-M17 specific nucleic acid sequence under said experimental conditions, each primer of the pair being at least 13 bases.

11. The primer pair of claim 10, wherein said M17 nucleic acid sequence is selected from the group consisting of SEQ ID NOs: 3, 30, 31, 33, 34, 35 and 36.

12. The primer pair of claim 10, wherein at least one of the primers of the pair hybridizes to a polynucleotide sequence which is unique to M17.

13. The primer pair of claim 10, wherein said at least one of the primers has a nucleotide sequence as set forth in SEQ ID NO: 37-40, 45, 46 and 62-573.

14. The primer pair of claim 10, wherein a first primer of the pair is as set forth in SEQ ID NO: 37 and a second primer of the pair is as set forth in SEQ ID NO: 38.

15. The primer pair of claim 10, wherein a first primer of the pair is as set forth in SEQ ID NO: 39 and a second primer of the pair is as set forth in SEQ ID NO: 40.

16. The primer pair of claim 10, wherein a first primer of the pair is as set forth in SEQ ID NO: 45 and a second primer of the pair is as set forth in SEQ ID NO: 46.

17. The primer pair of claim 10, wherein two of the primers of the primer pair hybridize to a polynucleotide sequence which is unique to M17.

18. The method of claim 4, further comprising quantifying an amount of M17 in the sample.

19. The method of claim 5, wherein said analyzing is effected using at least one oligonucleotide being at least 13 bases which hybridizes to said M17 specific nucleic acid sequence to provide a detectable signal under said experimental conditions and which does not hybridize to said non M17 nucleic acid sequences to provide a detectable signal under said experimental conditions.

20. The method of claim 4, wherein said biological sample comprises a fecal sample.

21. The method of claim 5, wherein said analyzing is effected using two oligonucleotides, each of said two oligonucleotides being at least 13 bases.


SEQUENCE LISTING (647 sequences)

SEQ ID NO: 1 - DNA, 1181 bp, Escherichia coli strain M17
cccccagctcatcgtgttttccgttgacgacggttgaaatacgcttatccgggtctgattaatgaatcaggctgattaaccctatcggtcaggctctctcaaatgaacataaatatgttgctatactcattctgacttaagcggctgtgatatattcagtataaaataagtcacttataacagttcgataaataaaattcgcagcttccgggttgataaccatcatatatactatggcaaggatattaatatggcgacacttgagcaacttatcattttctttcttcttattggcgttggtgtcattgctcgtaaagtgggagttattactccaggaaacacgccccaactcacatctcttgtttttaactttgcgatgccagccattatactttccggtattacaactgaacagccacatatttccggaaaagatctttccatcgtacttacatccgcattcactacgcttattttactgattatttgcagtcgtatgctggcaagaattttacgctatgaaagagaatactatggtgtgattactgttatgacaacatttactaatgtttccatgatggggatcccaatgatatattctttatacggctccgaagccatgatttatatcactgtctttttactcccatataacttattgttcttttcatatggttattactgcatgaaggaccagagtggtaatactgaaagtcttaacaccatttatcttcccggaaaatgagatgcctgcaccggtgaagacgacatttgcccccttgagtgcttccgcaatgggtgttcccatgaatgaatttctcaaactcacgcgaatcccaatcgttatctattatggcgattttattgctgaaaaaccggatactgccgttggtccggataaatggagaagtgaatatgaaatggcaaagcaatttgtcatgacagtcaatcgtcacggtggagacgccacccttgtccatcttcctgatattggtatcaaaggcaattctcatttcctgatggcggaaaagaataatcaggagattgccgggatactggcatcgtggttacaagataaaggactcgataaataactcagcgccctaaaagcattgatggagaggaatgaatattatgcgccttttacttatagaagatgaagagaaaacctcgacgtatat

SEQ ID NO: 2 - DNA, 170 bp, Escherichia coli strain M17
gttttttctgatggagattcagagcctcgaagatgtttgctctgttcactgctgtatcaagctttcaacgttactgaagcgctggccttcgccgtattaaaacgctcactggcctacaggcgtgcggtgaaaaaggtcattatgcccgcgcaaggtccacagcaaaatgt

SEQ ID NO: 3 - DNA, 1681 bp, Escherichia coli strain M17
gtagtatgtaaagaattacctttggttgatttgtaatagctaccgattacgttatcggtagctataagaaatttttatgtctttactgtgtttatttattcaatactagttggactatagcaatgaaagataacagacaaaaagtcgttcattataaaaaagcagtaatcccacacagcaaatcatcattacaaaacatactacttagtattataggtgagggcggcgttgcagaaaaagtacttagtagacaagaaaaaataacaccttcagatgaaaattcggggtatcgtttcttaaataaaagcgacacatacaaaactatactttttgggcaactggttttatttgaaaaagacaaaagccagtctctattagagcttagtgataatgttaaattttatgacattaactccatcacgtctaatgatataattattgatggagataaacgtagtgacaaaaaaagagagtttgtagattcaattttatattttggtgtactaaacaaccatgtcatgatcgttcaatccacatcgcttcgagcaagggagcttgaggcgcatttgaattggctaattaatacattcacagatgaaaaagaatctgtgttaatgcttcaagataagccatccgaagaaacgatgaaaaaaatggagaaagcaccagccaagagcataaaaattggtggattacccattacttcagaagttaaaaattcagagaaccaaacaccctccttttcaaatggttcggtgaagacaataaaataccgcccaatagggaaaggaggtgcgctactaaaagcattttttggcgataactgggcaaatgatataaacattaaagattcattagatgaggccaatttacaagtaagcctcgagattacatacttcagaaaaacaaatgatagcggtcaagctgttatagacagtattgctacctcactacgacatctagatgaggatgatattagcatcaaacttcaaggcggtggggaaataacgggaaaagagataaaattatccggaaaaattagcgttgaatataacaatggcattattgatgaaaatgatttattcttaaaaatgcataagtggctgttctcaaaacttgatgccggagaattggttgcaaaataacctaccaaaaggagggcgataatgaaatcgaacatcatccaccttattggtatgttggtcttctattttgctggttactttttgttcaagtcagcattgggctatattacaccgggtgttgtcctaactgctccgtgggctgtggttggactcatgcaattgccattatcatattgtatacaagccatttttaaggcaaatgaggccaatgaccactcgtctttaacccctagtgaggttaggcgattagtgccaatagtcaaatccaagagactgaaattggttttattattagctttctatgtattttcaactttgtttgtaatcttggggttaatagcaagcacaaatgatcaagccctactcttcagggtcttaaaaatttcaggtggattactctttatttctctctataccagtgtattcgttcataaaataatggttgagcttcaagagtttaaagcgttattaaaccgaagagaatcagagaagaaggaaagagagtcactccttgatagattaaaatgacccccctc

SEQ ID NO: 4 - DNA, 189 bp, Escherichia coli strain M17
taacgtaggtaaaggtctggatggtagtgcacgtgtcacactgaaaaactccagctgggatggtgatgtcaataacgccaaaactgctgtttttctgacggatggttctgtgtggaatggtgctgtttccattaacgcgccagtggaccgcgcatcagtaccaatgggctcggttcaaccaaataaaag

SEQ ID NO: 5 - DNA, 416 bp, Escherichia coli strain M17
tattcttttgcttcatcatcgccattaatcagatacaccggatatttaggaatgacatccacaagaatctgaatggctctctccgtgacgctgccgttaatatcgcgcgtccatcctttataatcgctctgaaattctcgggctgcctctcgcaactggtactgagcgttcgtcgggtcaaggataggtatgccctgattactttcatcaagtgtgccattgcctttctgcttgttcacccgctctttttcgatattttccatttgttcttcgcgcgcttgggcctctttatcaagtccatctttatccagttgtggcgccttaatataaaaaggttgtacaagaagactgttattcgcatagcgaccgatacgccaggcggtgatccagccgatctggtcactaattgcgctttc

SEQ ID NO: 6 - DNA, 555 bp, Escherichia coli strain M17
gatgggtattagctcatatcagaactaatacccatcaatgacacagagccaactacccaccctgtgtcaggggctaacttttaacaccataattcaccgctagataaaaactgtattgcattgctcgagcggcgcagcattatcagttagagtgtctttaccttgttaggctccacagttcgcactgtccaacgcctgtcactttcacacgctgcgccttcgagaatattcatttaatcgctgataacgaacatcttgctccagttgtggagcagcttgaacaatttgaagcaagcattgccccaactctcgaggcccccagtgttgttcggcaatcacggcattcagcagggatagagcgtccgcaggatatcggctgcaaatatctgattttagcaaaaggcgaacgtcgtagctaaggtgttcgagcggttgcagccagtcctgcaccactgccaaagccgccggaaattcacctcgggcagcaatcaccatacgagtcaacgattcggatatgcgtggggtggccaagttgcgggactttggccaaacctg

SEQ ID NO: 7 - DNA, 1357 bp, Escherichia coli strain M17
aagtagcgaattgctgccggtgctcgccaagatcagaatagtgattggcgctctccaaaaaatctgacttgaaagctatcagcaacggttcatacaggcgtggcgaccagaggaagccttcccacacagcttttgcttcgacgggattactccaagcaaacaagggcaatagatactgttcggtccaaggtcgatctacgcgaaaaaatgcgatcagccgcgaccccagcaacacccgaccatggcggaatagctctatctgtacattacacaatttggtgaaaagtgttttcaattcaacaggaagcaaatcattgtcattcgggttctgtttgaaccatagggtgatcagtgattgcgtgacatgcccaatgggatgattgatcgccgtagaaacaggatcataggtctcaattccgtttcgaatggtgctagactctgggcttgtttctatcatcagaacccgacgacaaagggccagtagaatctcctcgtggcagaggatggtcttcgaagcctcctccatccaccaagtgacagcgtgggaaatctcctgaagtactgcgtcaggcatgtcaagcaccaacggtgcggcgtaccgccacgaacgcaaaatcatccctggttcagcccaagtctgcagagcttcacgccaccgaccaacaggccacacatcatcttgtgatagtttacgtaacgcatacagactgtgaaaaaagcgcgtacggcaaacatcactccaagtgtcctcatagaaaggcagtctttctggcataggctttgcgagccattgcactaattcctgccacttacggggcgcaatgtcgacatctatactctcctcgaagcctggatcaccggttccgctcatccagtgagagaattcatcacgctcgttggttgccagttgccattttgggtatgctgtggatatttccgtcaaacgtgtagccgcagactctcccaaaacaaggcccgctcccctgagcttcgctagacacaaccagacggaatgagccaccaaataatgccacctgtctgcttccaaattatcctcgtacatctcgcgcggaggccctgccaagatagcagtttccagacgctcttgtgcaattcctgtcagatgtcgtccctgcaaaacaaacagtctgaatacctctcgcccagtatccgtggcccacaaccaccatgaaccgtcctctaacaaccaattaacccaccgctcaggtggtatgcagttgtcttggcttgcggcaaacagtgccagacgtttgaaggtgggatatggcaactcaaaccaattctgagcaatgcgcgtggcctgatcgctgtctttggctcgaacggctaaccatga

SEQ ID NO: 8 - DNA, 795 bp, Escherichia coli strain M17
atcttccaacaggtatggcaaggacgatttccatgactcgtcagcaaggtcgaacagggttgaacgtacgtaatcagcagtcagcaccagctcccaatccaccaattgcttgattcgcaagggttcatcagtgctgctcgaatcgtcttcactatagcgaaacggccgcctcaacataaccttgggagaaagcaactcgcgtaactccaagcgcaatgtagtcgtcaagccttcattctttaagcggttttgccaacgatacaaatccaggttctgcaatggcgatttcacacgaccactaagcagaagccgccataaagtagacataggtggaccaggaatagccaggggggaatgcaagagaatttcatctaactcagaagtcttacgctcccgcatcagtgctgctaagcgatctagttcgctctcaatcagaaacatccaacggtcgtgtatttgtccgccgcgttcagcaatccatatgatcaacctagggtcgcccagataacgaactagccaacgggctatatgggacattacgtcatcccatttgctagcactgacacatccagaaaccagcgacatctgcggggccagctcatagggcgcaggacgctgaaccagactgaatcggagtttcgggtcaatttcgacatgcggagatacacaaaagcgtggcagatcgctgtatttaaatcgttcgtccgagaaagctttcaataaccaatccagcggcggagcaggattgagttccgcaaagcgttttgctggtaaacctgatttatctgacaaggc

SEQ ID NO: 9 - DNA, 337 bp, Escherichia coli strain M17
tacataggcctgaaaagcctggcctgtacgtttagctgccacatgaaagagacggtcaaagttggtagtgactaatcgaagggcaccctcgcggctacgggctaaacgtaacagcgccgcctgagtatcaatagcgcccctacgacggagctttggcttaagggctttttccaacgcgcgtcggacggctatacgctgccctggtaagcgccgttccagcaaatctaatgtgccgtcaaattgcccacgctcgaaaacctcacgctcaatttctgaaagtgttgttccgttcctctggtaaattagttctaccaaccctttgaaaccaggtaaacca

SEQ ID NO: 10 - DNA, 526 bp, Escherichia coli strain M17
ctttgattttcagttgttagaaaatgcagggatccagctaattgatggtgacagaggcaaaaattatcctaaacataatgattttatggagaatggctattgtcttttcttatccgctaaaaatgtcactaaatctggttttcaatttcaagaaacattatttatcaatgaaactaaagatcgtgaactaagagcaggaaagcttaagtacggtgacattgttcttactacccgtggcacagtaggtaatgttgcgtactatgacaataataatccatataaacatattagaatcaactcaggaatgattatcattcgagctgataataaattatggaaccctaaatttctatatttcatattaaaatcagaactattaaaagaacagatcattaatcttatctctggctctgcagtgcctcaactacccgcgagagatattaggaaatttatattaccagttattaatagaagccttcaaaataaaattactaatattatatcagatattaatgataaggttaat

SEQ ID NO: 11 - DNA, 464 bp, Escherichia coli strain M17
ggaggctgggaaacaaaccgactggaaaatattttagagctggcttatgggaaagcactcaaaaaaacagaaagaatcgaaggagattatcccgtttatggttctggtggggtggatggttcccataatgagtttcttgttaaaggacctggtattattgttggaaggaaaggaactgttggttcattgtattgggagaacaaagatttttacccaattgatacagtattttatgtaaaacctaaaaagtacttctcgcttgtttactgctatcagctcttgaaaacactaggattagaaaatatgaatactgatgcggcagtccctgggttgaacagaaacaatgcttatcgtcttgacgtcataacaccaacacaaacaatcattgcccaatatacaaatatagtacaaacgttcaggtataaaatggattcaaataataatgagattgacaatctaaccaa

SEQ ID NO: 12 - DNA, 479 bp, Escherichia coli strain M17
aaatgagcgagttgagttatctggaaaagctactcaaggggtgtgatgtcgaatggatttctctggggaatattggcaaattcatccgtggcaatgggctacagaaaaaagatttagttgatagtggttttccagcaatacattatggccagatttatacaagatatgggttatcggctgatagaacatttaattacgtatcgccagagctagccaataagctaagaaaggcacaaaagaatgacttactgctagcgacaacatcagaaaacgatgaggatgtcgtaaaacctttagcttggctcggtgataaagttgctatttccggagatatgatgttatttcgccatgagcaaaacgctaaatatctcgcccactttttccagtcaaaaatatttcaagcgcaaaaaatgaaatatattactggggctaaagtccgtcgggtttccagtggcgatctagctaaaattactattcca

SEQ ID NO: 13 - DNA, 396 bp, Escherichia coli strain M17
caatactcgaaaacaagaattagtttcgaactacttgatgaaaagaattatgttggcgtagataatctgctgcaaaacagagcaggaaaatcattatctaatcatgtaccgacatcaggtaaattaacggagtttatacctaacgatatattgattgggaatatccgcccatatttgaaaaaaatttggcaagcagattgtatcggtggaacaaatggcgatgtattggttattcgttgcatagatagctcaataaataccagatatctatatcagattttggcagatgacaaattctttgaatacaacatgcagcatgctaaaggagcaaaaatgccgcgtggcagtaaagaggatataatgaaatacccaatccctgtaccgccactcacctgg

SEQ ID NO: 14 - DNA, 673 bp, Escherichia coli strain M17
aacgcctcttatagggcaagctgcatcaaataaattaagctaccttggtattgggaaaaagatatcctttgatggtgatttttatactgtagatggaatgaaattcagcaaaagttattatgagaaattatgggagcaaggcagacctgcgccattcgtgcaagcaagagaagttttgaatagcaatccaaaaattgagccggatcctagaggcgcacctggctacttaagatatgaaggtgcaggtcttgaaatgatctataacccgaaaacgggacaagttggtcatattcaaccagtcaaggttaaataaaaagaggaagtaaatggatatttggcctgaatttcaacgagatcttgagatgtatcgagatgttgttttatcaataaaacgtaaccttcggttgtatgaggagtgtattgaatcgttagttcatcagatcggaagcaccaattttgataatgcgcagcctctatttgatgacctctttcgtatgcaatctgaacttgctactatgttatacaaatatgaatataagccaggcaaaaggatacaagatttaatatatcatctggacagggatgatttttactcaagaaaatattggcataaaaaattcagtgatggattggcatggcctgaataatagaagtagaaatcccgccctcagtt

SEQ ID NO: 15 - DNA, 806 bp, Escherichia coli strain M17
tggaataccgggggggatggggaccggataatgaagaaaaagcccgtcatcaggaggttcagcaacaacgatttgatgaactgagtaaaatatatgataaaagtcacccggccggagagttaacggtggatgggcagactatcaggcagagttcggtaagtaaccgttatggtacgacgaaagtgtttgaaagccagactttgacggataaacagatccacaattacgcacagcaactggcaggcgatacaccgctgaaagaggtaaaatcgggaatctacacgtctaaactatcagatggttcggtaataacacttagaaacatatctacatcagaaggtcagacaggcgctcgttggactatagatattcgtaataaccaaaagcttactgaactggggaataaatattcacgtgtggagattaagttcaaatgattactgagttaagtgaaagagaatacaaggagttgacagacgctgcagagggacagactcttgatgctattttgtgttattcagttcctgatattcaccctgcagagtttagttttgcagatagaagagaaatctttttttgggcattaacccgtcttctgaaagaagggcgtatcaaactcgccaagcacggtaagtttttggaaggaagtgttgacgagcaggttgagcgttttcgacaggcatttccgaaaacagaagaagagatggaagacggtatctggttttttgatgagagttgtccggggggggctgtttgggtgcttgaagatggctctctcgaatggacctgataaccccttcacagcg

SEQ ID NO: 16 - DNA, 348 bp, Escherichia coli strain M17
aacagggggctataacgtacctggaggcataaacccatgctgttcaagtatacctctggcaacaggtgaaactaaaaagtcggcaagtggcagggccttatcggtcagacaggcaaatccatagatagcaatcggattataaggttccggtatttctataacttttactgaatcaattgattgcaagccaggaacataattggcataaccgatgaacagatcagtataatcattttcaattaaccactgcgctgctatcgcatttcctggaagaggcaacgaatgcctccccccaactaatgctacagcccgttgtcttattttttcaccgacactgcccatattttc

SEQ ID NO: 17 - DNA, 286 bp, Escherichia coli strain M17
tgttccgattcgcaatgtttcccgatttaataaagaataccagtcgtcattctcattgaatttattcgcccgaacatacaaacacaaacgattgctggcaaaaggtttaatacacaatgctcgtcctgcggacattaatatctgtgggtgagtcatattggctgatgcaaaaaaatcgcatgcctcacccgcctcaatacgctcccttaatatacccgctggtccaaaatcacactgtatattatcggcctgatactctgacacaagtgatttccatacccggcgt

SEQ ID NO: 18 - DNA, 506 bp, Escherichia coli strain M17
ggtgaaagttcgttcagcttgcgtagtaaatgataaagaaagcaaaaaaatcagtaataaaaacaacttattatttattttcatattccatcacactacaaatcggtatataaataactatatatcgaaatggtagcaccagtcaggctgtgggaaaagcgattccggtatatttttgatgtgaccgtatgcagagaatgtgacgtttatgtgttcagcagaggttttcaattccgttatctgtaaccctgaatacgcatgatcataatagcaaagacaaaggagtcgttacgttgccgcaccctatccattcaccatcgcctgagacgataaaatatcagtaccatcagcttgtacataaacggattatactgatttcaataataacgctggcgatgctctcctcactatttctggacatcatactgggctcttccaatttgccagtcagtgagctatggcatgctttagctcatccgtttagtcgtgataccagcgtccatgtc

SEQ ID NO: 19 - DNA, 163 bp, Escherichia coli strain M17
attttatcaggcgtatttatcattgtttttttatggtcattttcccgctcatggcaacttacggcattacgcatgggagaagaaagagcgttaagttttggtatcaatgcacagcaactgcgtctttcctccctgttacgtataagtttgcttgcagcattat

SEQ ID NO: 20 - DNA, 402 bp, Escherichia coli strain M17
attagtattattctgcgctatcgcaggagtttgttatgaaaggtctttcactgcataaattaaatgcaggttatcagaaaaaaaacgtaatatgtgatctcagtttacctgaactgcaacgaggaaaaataacaatcattctgggaccaaatggttgcggtaaatcgacattgttacgtgcactggcgggactgaataaggccaacggtaaggtatttcttgatggtgtcaacctaatgcaaatgccattttctgcgcgtgccgacagtacaatatttttgccacagtttttaccacatgatgtgcatctgaatgtgttggaatctattctggtttccctacgcgcgtcaaagatgggcgggaaacatgaaaaaacacaatcaatagctattctcaaacaac

SEQ ID NO: 21 - DNA, 1092 bp, Escherichia coli strain M17
tttagtaaaaagtgagacacgtaatcgcaatatagtcaccattatggtagttcacgacttaaatattgcactacgatatggtgattatgtcgttatgttaaaaaaaggcaaacttattgccagtggaaaacccaaagaggtcattacgccagagaatctggctaatgtttatcgcatccgtggacgaatagagcattgttctcatggcttgttacaacttatcgttgatggtgtcattccggattaacaaaatggatggaagatttctctcccggaacttctcatcactttttgacaccatcatcaacgatgattaactaaaacagtgttccctttgatgatgagtttgtttatatgcagcatgaggattagctcgtgctttatatcagaactcacctcatataaaaacgagtaattttcaacttttcaaggtccaatgcaaggtcaatttacaatgaatgccctgttggcctaaaatacacccatcaaatactatttttgatcacttgtataacggtttcgatacgctcttttatttcctctagttgctgctgacgaccttcaggtgttaatgtctgatcttcgttcagatccaaaacccatcgacgtggaatcaatgcaccaaaaacaatatccattaacatccccacacaattccccgtatttttaacagtaatgcgtccttgagcttgctgaatatccaaccactcaattaacagttctcttgagcgaagtgttttagttgtatataaatagtctgaaagatctggaaagagaatagactcacgcgtcattaattttaataacgcttctctctctttagcgtcttcctgacttatatctaatcggaatatgcgaaacagtgcttcattaagagggagctcttcattttccggtctcggaagatcgagaaacagtgctttatattccttaataacttcagagaaaatttcttttttattgctgaacacttcataaatcgttctttttgatacttttgcccggcgggcaatttctgtggttgaggtttgagcaaaaccaagctcaatgaaggcactgtacgccccaataactattgcttttttacgctca

SEQ ID NO: 22 - DNA, 561 bp, Escherichia coli strain M17
ccttacacttgtataactggtgattcttcttgagagtatcgactggtggcatctggagatacgctttcgttggcaggttatgacgcaacttaagtgggagaaaggcagtcatgctgcagcattgacgatttgttatcactgaatacttggggttgtactatagtgcttagacgatattacagattaatttacggacataaatatctcaaaatgacaatttaataaaactcaggtaaaaaatgcattgcggttttaatcttttttgatatataagcacatgcgttataaatataaaagattacactttgttattgaaataaattggcttggctattcaatatgttaattttcattatagggatgtttaaaaataaatgtattctgagtagtcatgtaatgtatgtgagatgattttaatgaatgcaaatgtgtaaataataagaaaatgctttttgatgatggaaacatcacaaaacacaactagttaaattgctacattccccgtttgggtgataatgtctgatttttattagcatcttcaacttattaagttgatctgtg

SEQ ID NO: 23 - DNA, 279 bp, Escherichia coli strain M17
ggttatccgtcaggaagaggaaaaaatgttctgcctattctctgctaaatttattccacaggttcctagtggaacgccgtttacatgaaaaggttgcttgacgctctgaagcgtaatgtcttgaaaattaatgttcaactattatttcatttatcactgttaggttgggaatgaatcaaactgacagggaattatatggaaaatcaacccaataccggcttcggataagtatcggtttttaataccagcggaaataggtaaggtcaaaaaacagcctta

SEQ ID NO: 24 - DNA, 1192 bp, Escherichia coli strain M17
gccttgcttagacctgtgtccaaattacgtgggttggatcattgttccacgacgatttgaaagtatattaatgaacaataaatacacatttctgtattttcgtatattttacaatagtgaatatacaaaatctaatatattctatatttttttgttctaaccttttgaaatgaagtgactttttattctgtcttatgggtgtttttttctgctgcaagcacttccagtattatttccgaatttacattataaacgcctcatgaaattatactttcataggaagtggaggtggttatatgaaaagtcaaagtgctttatataaactaaacctttctgataaacgggccattttaaatataaatagattagaaatcagggatgttcacttttttgaatgtacaattatttatctctatgatgcacgaattgtattaaaaaatagcaatggtaaaaagacaattattttccctcagtcaatttgttatattgaaaaaaatacaactgtagatataattatatgtgataaaaaaagggaaggccttatgatgtctataatgtagatggtgatatactcaataatatttgtgagataatgagtcctcttctttcccattttggaaatgtaaattatccatatgctaaggtgttctcatttacagcagatgatgttgataaaaaaatctttcatcttttaatgaaaaaggaaacgccatattacaggaagttatataaacttacataccttttatcaaagtgtgataatattgagacattaatttattcattatcaaaatcaaaaaataaaacaattactgaaagactaaaggatataattgagtcagacctatctaaaacatggcaaataagtgattttgcaaaaattctacatatgtcagagtcattaattagaaaaaggctcaaaggtgaaaatataaactataataccctgattgttgatattagaatgaactatgcatttaacatgttaatggcaacggaaaaaaacataaatataatatcaagagaggttggttatgtcagtacatcatattttattagtaaattcagaaattacttcggaattacaccaaaacagttttcaataaaagtaaagaataaaataaggagttagtttctataagacatgcacttaaatgtatccattaataaaaatggtaatatgttcaatcag

SEQ ID NO: 25 - DNA, 777 bp, Escherichia coli strain M17
acgtggtttaaaagttatcatgaagaaaatccctgattagaaaggtgctcttgaacatcatgaatgtaaaagatttatcaattacaacggttctcttggccgccctttattttatggctttttctttttacaaaggatacagctcattttatggattccctgtaagttttatttctattgggattgaggaaatagttaagttttctgttattgcattaggacttttgtgcaccttagtcgctttgctgcatattgatactgaagagaagaatattccatggggggtgtatttatttttcttcattttggcctctttgctttcatactggacaatgtatttatatggcggtagctcatacctttatgaaaaatccataagggatgtggctacccaagcaatactattgggatttattagccctatcagtgtcagggcagtatctgtttttattaggaatgaatttagaataaaaaacaaaactcatggggttatgttttttatatccatagcaatgcttccttcagtccttgggtgggcatgggcatacttatcagatgagcctcttttttattctaaaaaatatgatgcttatattatcgaaagctataacggaaaatttgtcttggggaactgtaaaaaaaaatagctctgaatttatgagtgtcgaaggaattgaggggaaattaattcctgtaagcataaaagaaacacaacaactaaaagtatgttttttaaaagcttctaagctcaagaaaaatcgctcagaatgattgtct

SEQ ID NO: 26 - DNA, 1437 bp, Escherichia coli strain M17
aaaatatcctcatatggtatatggttaaatgctagatatcatgaattaaaggagacaactaatgactgtgggtaataagagacagcctaatggattacctgaatattattgtcaccgtggtaacgacataaaagatgcgatagttcgggattttattattcagcctgttgcacacctgaaacttattactggacaaactaaaaccagttattccggttatccgttaaccgatcagtactattgtttttcatttaaaaaacgctctgatgaaaattgtacaggggcttttttctgtggtagagatgttgcaaaggatttcattcggcgtatcggttgtacagagctgccgttatttaatcctatgaaatcggataccagtgcagcaatagaacgttccggttctggctcaggtagttctggtattactgtcaagccaaatgctgacaaaaagctatttatagacgcagctcgtctgttaatgacgctgtgggattcaacaagtaagggacaaaccccactggcgtcggttcttaatgaagcaatttctggtggtatagataaacctccagaaattagacatgtagtggcggttaatacaatgttgggtaacacaaaaaatactttaactggtttgctggataaagcaagagcgaagtatcctgatgtgttattcagagattttgatttcacgcctcttaacgagattgtaaaacgttcaaaatcatataataagcatcccccttgttattgagtatcagacactgtattggtaggaaggcctatcatttcatttaatgtctctcgatgtactgctggtgctaaagggactggcggtgcgtctgttggaagcagttctttagcttcgggccatacatccagtaatcgttttactgtccgaactttatttagagccgcgctgacgttctgtatgatttcggtttcatcacttttaatttgtgtgtagagagcatcaaagctataaaactccgttaccagaggatcttcgccaagtagagtaaattctgcaggtgctattttattaatatgctcgggggctcctgattcataacctctgtagttgccattgaaataaatgtttgctctggctccagccagattgagatatatgcagcaatcttttcgaattaaagaactattggtttttaatgaatcagggatttttgttaccatggattcaatcttttttatattttttaatatttcagcttctttctctacgcccccgatagctgcaagccgaacacgttcggcccattcaacacgagcagctcgtagtgctgatttacgtttggggatacccgctttctcaagtgcgttattgatgatttgagttttaaggtgattagttaatacgggctgtgacattattctttccctcatgagataccccagcacgtagctgggggatgaatattt

SEQ ID NO: 27 - DNA, 418 bp, Escherichia coli strain M17
tggctttggtgtctgaagctgtgtttggcaagcagggcattcacctttgagcggtttatacgttttcacgccgtttcctcattcagttcactttggtggttcggcagcttcgacttgtatcagtagacagtgcttcgccgcaccccaaagggaacttactgacccgtattgccgacatcctgtcccgccacggtcccgacgcatggtttagagtcgcgccgttcgacttgtggctaaagatacaatttgagttgtattgtgtaaaccacaaatgtggtattttgtggggtgtgaatactactttattgatatttaagtgaatttaatttgtaagactttacaaatggggtaagggaggctgttacgcctcccgatatttacggtgggaatactaacgtttacggcggtaaatacggtg

SEQ ID NO: 28 - DNA, 719 bp, Escherichia coli strain M17
tgtctccttctttaaactctggttgcatgctgtcgccatcgatacgtaatgcgaagcatgtttctggaatgtcagagtctgccaaaatatattcaaattcccctgtcagatctgttatgtctcttgcttctgtaagctctcctgcctgaacgtaacttaatataggtatgcgtctggtacttatctccgcgagaggcataatatttttaccgttcagtagccagtctgggctacatttcaaggccttagccagatccagaagattgcgtggctttctggttcgtccactttcgatggattctatggattgttggctaacccctgcagaattcgctacttccacttgagtcattcctaattccaaacgacgtgctttaaaacgggctgcgagagacatgggattagtccttctgggttgtattaatgtgtatctccacattaaaaacaatttttgttgtatttgacaaacctcgtttgttgttgctaaataccactaaaattgtatgaggtgaaagcaatgaccttagcgacccgagtaaaagaacgacgtaaagaactcaaaatgacgcaagttacgttggctgagctcacaggagtgagccagcaggcaataaacagaattgaaagtggtgttattgctcggccacgttatcttcttgaaatgtctatagcattggattgcgaccctaattggctgttgtatggctcacaaaacgata

SEQ ID NO: 29 - DNA, 234 bp, Escherichia coli strain M17
gcggatctgtatacgcaatcggctttgcaaaggggatgagcaatgaggggtattatttttctgttagctgccttttctgcgtgcagcgcgtgggcggatggcttcacggttaaatgcggtggctacactatggttgcaaaccagggcgagttatcgactatcaacggtgaaagagttacctctcaaaaaatcaccgaactgggtaccaatggtttgaaagtagacatggggctt

SEQ ID NO: 30 - DNA, 937 bp, Escherichia coli strain M17
ggctgggcgatgatttttgacgtccataatattatggatgaactgttgccaattgcgctaaatttaacaaaataatactggttaacgcattttttgaaccatctgtacacagtagttgtctatactttaccttaaaggtaaactctgcgctccactgatgttcactatgggaggggatatgaggaaaatcgttaatcgttacttagccgctgccggtagtgttatggacattatgccgactacagattactggcaatatgctgcttctggatctgattgtcgggcaataagaagtgatgttaaagtcattgcaagtgacatgaacagagttatcaggtatgagttcaaaaccccgcaagcaggcggccgttctcaaaaagcatcaggaaaacaaagcgtcgcaaaaagccgttcctgattcggatggtatcgacgaagatattgattcctcggaatatgatgagtctcaggaagtagtggaaaagctgtctgcagttgccaaccttgatcctgagctaaaatcgaagttaacacctgaacttcttgagagagtttctaagaacccgaaggttattgccatgtttcagcaggagtcttttcagggaccactcccacctcctgctatgctcttacaatataatcaaatattacccggcggcgctgagagaattttcaggctaaccgaaaaagagcaagagcataggcataagacgcaaagtgatgcattacttggtgcggtttcacgcgacaaacgtgggcaatggatgggattttgcattgcaatggtagtgcttataatggctgcgttttttgctaatcgcggtaacacttggttcgcaggtactcttgctactttagatctcgtaggattaactacagtgtttgtttatggtaggaagaaacctaaataatccatacatcctttcaaacccgcttc

SEQ ID NO: 31 - DNA, 888 bp, Escherichia coli strain M17
gttggtactttcattcatatacatgaccgttgtatacggtgaaagagtggcattgataaacctagcattttcttcaacacgtctttgttcaactgtataataataatgtttttcaaggatactctcggctacttctccccaccatttattaataggttcattattggttaaattggggctgcttaatgaggagaaatttgcgtaacgtccccgtcgggcatcagcaaaggaatctaaacattcaagtattttgtaggtaattttatgtgtaggacgtgaatatctcagagatagttgcatttttttagaaatactatcaacctcgttggttaattcaatgagcttgtgtccaaattttcttacaatattctcgtcaggcatttttcctttgtgttcaatggcatagtgtgcaacaagtattagtttagcaagtctctcaaaacctacagaaagcccaaaaaatgctgtgtaatattcacctattttgtctgcataattggctttacctatggctgttgccccagaaccaatgagctgtgttactaaagatgcttctcggcccagagcgtgccatgctggaatatcccagggatttttattcactttattattttcctttgctcataaatttaaattgtttgagtatctcataaaatataaggaggtggttaaggttgttgatcgcatataatagctattgaaagtcaagaactcggttttatgttctaccactaattaatatattgtgagtctgacaatatattaattagtatgatcaaaccagccatcaactgattccttgtatctttcagagtctccagaacaaaagccttaacgaattccttatttgtgagtgcattacaaaaagccattgttgcagggaacgag

SEQ ID NO: 32 - DNA, 185 bp, Escherichia coli strain M17
tatcagagtaaaaatgtaacgctgatttttttgttaaatgatgcgcattatatattcaatgattaaaatactcttgatcaaataaaactatctaattaataatgtaaaatttcattatgctatttaagatgtgaaaaataacttaaataagggatgggatatatttatattatcccggttataga

SEQ ID NO: 33 - DNA, 2424 bp, Escherichia coli strain M17
atagctattttaagaaaagaaatgatgtaaatatgcatctggtttttctttataatgtaaggatgtccgatgaaacatcaccatcgtgaatgttatctggttaataattttcgtatgtaatattatctatgccatctggccatgttttcaattgttttagttctcgacaactgtttttatatccagtatgattttactcgtatccggcagaatatttttagatgctgtttcatgaactccggttgaaaggtttattctctacttttgggaataatagttaaaatggagataagtgtaatgacgtttagtaatgaaattgaagttttcataaaagattttgttaaagatcttgaaaatgattgtgctgctattttttctggcgcaggaatgtctaaaagtcaaggttatgttgattggaaagaattacttagcgatatagcagaagagcttagtttagacattaataaagaagacgatctaatatcgcttgctcaatttcatgttaatgaaaagaaaggggctacaaaactcacaaggaagataatcgaagagttttctgagcaagccgagccatcagaggttcataatataattgctcgtttaccgattaagacattttggacaacaaactatgacactttaattgaagatagtctgcggaaagtttacaaaattgcagatgtaaaaagaaacatagatgatttaaatacaacacgacctaagcgagacgcgatagtatataaaatgcatggtgatatttcgtccccatccaaggctattttatataaagagcagtatgaaaaatactacagaacacacgcttcatttatcaccgcacttagcggagatttaatatcaaaaacctttctattcatagggtttagttttaccgacccaaatctggattacattttgagtcgtttgtatactcattctggggacaagaggacacactattgctttatgaagaatgaggctaataattccctagatgatgaagaaacagtaaattataagatcagaaaacaaaaactgcgaatcgatgatctcaagagatttggtatacaagtattattaatagatgaatattctgacatccctaaaattctagctaagatagagcaatcatttaaaaagaaaacgatatttatttctggtagtgcagaaaaatatggggatcttgaaagacctaaagcattagattttattcacacattagcatatgagattataaagaataattataggatagtgaatggatttggatggggcgtcggaagttctgtgataaatggtgcgttggatgcaatttatagtaggccagataaattatcagaagaacagctcattgttagaccatttccacaaactgtcagtaaggggaaagatattcaagaactttgggaggagtatagacagagaatgattcctcttggtggtattgcattgtttatttttggaaataaacaagaggaaaataatgatatagtaaatgctaggggagtatttagagagtttgagattgctgttcaacatggccttgtaccaattcctttacccaataccgggtatatgtctaaatatatatttgaacaaatatcacaagatcctaagaaatattatggaagtaatgaatggatatataatgatttgtctgagcttactgaaaaagatttttcggtaaaaaaaacaatagaagttgtaataaaaatcataaacaaactgaataaataatatattaggtgaagttatggctcatgaaacattcatatcatataaatatagtgaagcacaggatttaagagacaaaattgtcaaggcattaggtgatgatgctgtttactataaaggggaaacatcggagtctcctgatataagtaatacaacagtagataatataaagaacaatcttaaaaatatgattttcgggacatctgttttaatcgttattatttccccgaatatgattaaaagtaaatggattgactgggaaattgaatatgcattgaaagaatacaagagaggaggaaccacttcccgaactaatggtattgttggtgtgataatgaaggttaataatagctatgactggttagtatcttcttcaatcaagcctgatggatgttcatcaagaactattgacacaactaaactatatgacattattaataataataggtttaatttaacttcagaagataaatattgttgccctgagtgcaaatcatatgatcaactgactggttcttatatttcattgatagaagaagaccgatttttagcagatgtatctttatacattgaaaatgcctatgagaaatctaaaaaattaagttcctacgatttatataaacaaaaatagatgataacggaaaagggccacaactatttgtggcctatatatacacatattaatcaagcagg

SEQ ID NO: 34 - DNA, 1407 bp, Escherichia coli strain M17
taactaaaaggatgctggatcaaaatgcccctgctacgctgtggaaagtgacagaaggggcatgaattgtatttcatttaatgtggtgaatgatattttattatatcgatttgttagataccttcatcaatttttcttgagtttttcttattctttcaagctcttcaacaataggatttttctcaccttcactttgcatgaaagggcttaagtctatattgtttttttcaacaacttctttattcatataactatacgttgcagttattgtaaattgttgtggtaaagggtttttggattctccaaaaataacccaaccttgaccaagggcgaagaataactcttgattgggcgctaagctatcaatagttgatgtaaaaacacttgtttcttttaggtttttagttggttcatcgaactgataaaaatccttatctaattctaattttacattcaaggctggtgtcttaccgctattataaattttcaggtataggaattgcgagtgaggtcttaccattggctggattattatataaggacgagacattgcctcggtttgctcattcattatctgagctgttcgttcgcttattctcgacatcttgtatgtcaaaaatgcataaattcctgtaatgataacaagaattgctgtaagtatttccattgtcatgattaactcctaagtataatgattcataccagtaaggtataatattttaactattatttcattcttttctgttgaatagttgattatatccaagttccatttcttcaataaaatcttcactaaatgttggaaatccatgtataataggttctttatctcgaattttgaataagttataaagataactactttctttatatacgacatggatggcaggatatggcctattttttataatcttattgtttagttccttatcattgataatgttatattgatcccctgttatatcaactacaatattatctatctcaagccagtaatgggttatttgaccatttttcccggtagctgcgctaactccttttagctcacattcaggccatagggtcagaaaatggtaagaaagaagcattgaggtaagcttacaactcataacaggaaaacagtctataacaagactggtgttctttttatcacaactttcaattaatctacgaagcttaactgcttcttcttttatttcgataatggttggcaagatcagctccctgactacatgatacgccgagtatttaatatatacgatacataggtcattaaaaagtatgttctaaatttagtgtttcttatggctcggtggactcagttttggcgcgcttccttgcctcatgtgttctggtggcattgaaggtgtgagatctatgcttctattaacttccggctgttcctga

SEQ ID NO: 35 - DNA, 1449 bp, Escherichia coli strain M17
ctttacatttcactatgtgattattttttaattgtatacacattcttggattttgaagttgagattatccttcataaagaaggatgaagagttcccttttaaaaatgactctatgctatttatgtgcatatcataatcatcaatgagcaccttatcttgggatgaaccaataaaaataacattgaccttatctgcactaaggttaatgcaatgatgatttatattaaagtctaacaagtgagcaagagcataggttttgactggaaggttttcttcactgaaataactattttgccaagataaaatgaatataaaaattaaaaaagaaaacaaaacggttgcgaatgaactcacaggagggagatcgaaagcttcatctttaaataaaaaacaaattatttttattatgtgagttaaaaagaatgttgctagaataaacaataaaatagggttgatatattttaaaaatagaaaaccagttaaaaaagaacgtgtataaggaaaggatgatgaatcaatgccaaatacactatttaaaagtgctgatgattttgcggtgcaaaaaataatcaatcctgatatggctatagaggtaatacattttgtaattattgagttccatagcaccttaaaaatcaatgttctttcaataaatacacctaaccataatgtagaaaaaattattgataatagtatgcatgtaaattgccattgagagaaccaatcatctgagtctgtctttgctatacctaaagccaaacatatgagagctaaaatgcctatcacaattaaagcatatgatttctctgattcatcctgacgttttttaagctcatcatagttttgtaacttttttacaaaggctgttggcaggttgctcaattcggaagtgtcattcttcatatgttctccaagataacaacgatatgctggaagtataccgctgtattaaattagtgtgcaagcggaattatggtggagaaacattcataatatcaattttattgtatgtgtctatttgacgtatctctgtattgatgataactaaatataacattcaataaccaaaggagataaaaaacgaatgcacaactggcctatgtttttactattgatactcgtattcatcagaacagtaatgaaaagattactgaaattttcattgcgaaatataaagaaggaataacgtatcattttggccctgatatggaacgatttgagtcctgctttgaagcagcgctttttgctattggtgaatggcaaataaaagttgatgcgatgacacgataccatgaggaagatgaagtgatggagttctttagctctataccatcagatgaagcgggaaatctggcgacgtctattttgctgttcagtgatgttctcgccatgaaaggcgtggatgaagtatttgcctattttttgggtaaggatgatgttgtatgagtg

SEQ ID NO: 36 - DNA, 730 bp, Escherichia coli strain M17
gtagtatacagaattgattttgtattaattatttgaatcaaaaatgcacgacatagtaagaattaaatgtgttgtgcattttgatttcaacagcctactttaaccgaaaagaggagttgaacgatgatctgacgatttctttacagtttctgtgcgatatcccagataaaaccaaatgggtctttcacacgggcttttaaatcaccccagaacatttcagcaggttcatttagaacagtcgagccagcttcaatcatttcatttaccagagtgatcatatcatctacatacatataaaaaataaagggaggcatagctcccgaagtatttgggcttttcaggtcggaatcccatccttccttatttatggtgaagtttgtgccacggtaacggatacgagcaaataatacatcaccaccatcaccctcaagttcagaaattaccaccatcttcattacttcagtgtaaaaattaatagctgcttttacatcagggacatttagtgctgttgtcaaccatgtcagccccataccttcccaaggatgtgggcgattttctacgttattgaaccagtccagtttactcatttcatttttctccttaaaaacagcatattcatgtgaaacgtttctgtcgtatattactctaaattgataaatcttatacaatcattaagaatttatggtgtcagtttataaagggcggggactatttgtagctaattagaact

SEQ ID NO: 37 - DNA, 20 bp, Artificial sequence
  Single strand DNA oligonucleotide
tgcgaatcgatgatctcaag

SEQ ID NO: 38 - DNA, 20 bp, Artificial sequence
  Single strand DNA oligonucleotide
ttggtacaaggccatgttga

SEQ ID NO: 39 - DNA, 20 bp, Artificial sequence
  Single strand DNA oligonucleotide
gctgtttcatgaactccggt

SEQ ID NO: 40 - DNA, 20 bp, Artificial sequence
  Single strand DNA oligonucleotide
tggggacgaaatatcaccat

SEQ ID NO: 41 - DNA, 20 bp, Artificial sequence
  Single strand DNA oligonucleotide
tccgggttgataaccatcat

SEQ ID NO: 42 - DNA, 20 bp, Artificial sequence
  Single strand DNA oligonucleotide
accactctggtccttcatgc

SEQ ID NO: 43 - DNA, 20 bp, Artificial sequence
  Single strand DNA oligonucleotide
ccgtggacgaatagagcatt

SEQ ID NO: 44 - DNA, 20 bp, Artificial sequence
  Single strand DNA oligonucleotide
tttttggtgcattgattcca

SEQ ID NO: 45 - DNA, 20 bp, Artificial sequence
  Single strand DNA oligonucleotide
atctgagctgttcgttcgct

SEQ ID NO: 46 - DNA, 20 bp, Artificial sequence
  Single strand DNA oligonucleotide
taccgggaaaaatggtcaaa

SEQ ID NO: 47 - DNA, 144 bp, Escherichia coli strain M17
gctcataaatttaaattgtttgagtatctcataaaatataaggaggtggttaaggttgttgatcgcatataatagctattgaaagtcaagaactcggttttatgttctaccactaattaatatattgtgagtctgacaatatat

SEQ ID NO: 48 - DNA, 322 bp, Escherichia coli strain M17
atagctattttaagaaaagaaatgatgtaaatatgcatctggtttttctttataatgtaaggatgtccgatgaaacatcaccatcgtgaatgttatctggttaataattttcgtatgtaatattatctatgccatctggccatgttttcaattgttttagttctcgacaactgtttttatatccagtatgattttactcgtatccggcagaatatttttagatgctgtttcatgaactccggttgaaaggtttattctctacttttgggaataatagttaaaatggagataagtgtaatgacgtttagtaatgaaattgaag

SEQ ID NO: 49 - DNA, 263 bp, Escherichia coli strain M17
agtaaatgctaggggagtatttagagagtttgagattgctgttcaacatggccttgtaccaattcctttacccaataccgggtatatgtctaaatatatatttgaacaaatatcacaagatcctaagaaatattatggaagtaatgaatggatatataatgatttgtctgagcttactgaaaaagatttttcggtaaaaaaaacaatagaagttgtaataaaaatcataaacaaactgaataaataatatattaggtgaagtt

SEQ ID NO: 50 - DNA, 188 bp, Escherichia coli strain M17
taactaaaaggatgctggatcaaaatgcccctgctacgctgtggaaagtgacagaaggggcatgaattgtatttcatttaatgtggtgaatgatattttattatatcgatttgttagataccttcatcaatttttcttgagtttttcttattctttcaagctcttcaacaataggatttttctcacct

SEQ ID NO: 51 - DNA, 193 bp, Escherichia coli strain M17
gatcagctccctgactacatgatacgccgagtatttaatatatacgatacataggtcattaaaaagtatgttctaaatttagtgtttcttatggctcggtggactcagttttggcgcgcttccttgcctcatgtgttctggtggcattgaaggtgtgagatctatgcttctattaacttccggctgttcctga

SEQ ID NO: 52 - DNA, 20 bp, Artificial sequence
  Single strand DNA oligonucleotide
aaggtaaactctgcgctcca

SEQ ID NO: 53 - DNA, 20 bp, Artificial sequence
  Single strand DNA oligonucleotide
ccctgaaaagactcctgctg

SEQ ID NO: 54 - DNA, 20 bp, Artificial sequence
  Single strand DNA oligonucleotide
tggtgctagactctgggctt

SEQ ID NO: 55 - DNA, 20 bp, Artificial sequence
  Single strand DNA oligonucleotide
tgacggaaatatccacagca

SEQ ID NO: 56 - DNA, 20 bp, Artificial sequence
  Single strand DNA oligonucleotide
cgctgtggaaagtgacagaa

SEQ ID NO: 57 - DNA, 20 bp, Artificial sequence
  Single strand DNA oligonucleotide
aatgaatgagcaaaccgagg

SEQ ID NO: 58 - DNA, 20 bp, Artificial sequence
  Single strand DNA oligonucleotide
gcgataactgggcaaatgat

SEQ ID NO: 59 - DNA, 20 bp, Artificial sequence
  Single strand DNA oligonucleotide
attgcatgagtccaaccaca

SEQ ID NO: 60 - DNA, 20 bp, Artificial sequence
  Single strand DNA oligonucleotide
aaatcggataccagtgcagc

SEQ ID NO: 61 - DNA, 20 bp, Artificial sequence
  Single strand DNA oligonucleotide
gcaccagcagtacatcgaga

SEQ ID NO: 62 - DNA, 19 bp, Artificial sequence
  Single strand DNA oligonucleotide
ccagctcatcgtgttttcc

SEQ ID NO: 63 - DNA, 20 bp, Artificial sequence
  Single strand DNA oligonucleotide
ggtgtcgatgcgataaaacc

SEQ ID NO: 64 - DNA, 20 bp, Artificial sequence
  Single strand DNA oligonucleotide
aaccctatcggtcaggctct

SEQ ID NO: 65 - DNA, 20 bp, Artificial sequence
  Single strand DNA oligonucleotide
gttgctcaagtgtcgccata

SEQ ID NO: 66 - DNA, 20 bp, Artificial sequence
  Single strand DNA oligonucleotide
cctgatggcggaaaagaata

SEQ ID NO: 67 - DNA, 20 bp, Artificial sequence
  Single strand DNA oligonucleotide
ggtgtcgatgcgataaaacc

SEQ ID NO: 68 - DNA, 20 bp, Artificial sequence
  Single strand DNA oligonucleotide
tccgggttgataaccatcat

SEQ ID NO: 69 - DNA, 20 bp, Artificial sequence
  Single strand DNA oligonucleotide
tgacaccaacgccaataaga

SEQ ID NO: 70 - DNA, 20 bp, Artificial sequence
  Single strand DNA oligonucleotide
tcggtcaggctctctcaaat

SEQ ID NO: 71 - DNA, 20 bp, Artificial sequence
  Single strand DNA oligonucleotide
cggaagctgcgaattttatt

SEQ ID NO: 72 - DNA, 20 bp, Artificial sequence
  Single strand DNA oligonucleotide
cctgatggcggaaaagaata

SEQ ID NO: 73 - DNA, 20 bp, Artificial sequence
  Single strand DNA oligonucleotide
gcttttagggcgctgagtta

SEQ ID NO: 74 - DNA, 20 bp, Artificial sequence
  Single strand DNA oligonucleotide
atcggtcaggctctctcaaa

SEQ ID NO: 75 - DNA, 20 bp, Artificial sequence
  Single strand DNA oligonucleotide
gttgctcaagtgtcgccata

SEQ ID NO: 76 - DNA, 20 bp, Artificial sequence
  Single strand DNA oligonucleotide
tccgggttgataaccatcat

SEQ ID NO: 77 - DNA, 20 bp, Artificial sequence
  Single strand DNA oligonucleotide
accactctggtccttcatgc

SEQ ID NO: 78 - DNA, 20 bp, Artificial sequence
  Single strand DNA oligonucleotide
tcggtcaggctctctcaaat

SEQ ID NO: 79 - DNA, 20 bp, Artificial sequence
  Single strand DNA oligonucleotide
gatccccatcatggaaacat

SEQ ID NO: 80 - DNA, 20 bp, Artificial sequence
  Single strand DNA oligonucleotide
tatggcgacacttgagcaac

SEQ ID NO: 81 - DNA, 20 bp, Artificial sequence
  Single strand DNA oligonucleotide
ttccgggaagataaatggtg

SEQ ID NO: 82 - DNA, 20 bp, Artificial sequence
  Single strand DNA oligonucleotide
tcttattggcgttggtgtca

SEQ ID NO: 83 - DNA, 20 bp, Artificial sequence
  Single strand DNA oligonucleotide
gcagtatccggtttttcagc

SEQ ID NO: 84 - DNA, 20 bp, Artificial sequence
  Single strand DNA oligonucleotide
aaccctatcggtcaggctct

SEQ ID NO: 85 - DNA, 20 bp, Artificial sequence
  Single strand DNA oligonucleotide
gcgtagtgaatgcggatgta

SEQ ID NO: 86 - DNA, 20 bp, Artificial sequence
  Single strand DNA oligonucleotide
ggcgctcatcgtattgtgta

SEQ ID NO: 87 - DNA, 20 bp, Artificial sequence
  Single strand DNA oligonucleotide
gcggaataacgagtccacat

SEQ ID NO: 88 - DNA, 20 bp, Artificial sequence
  Single strand DNA oligonucleotide
agagcctcgaagatgtttgc

SEQ ID NO: 89 - DNA, 20 bp, Artificial sequence
  Single strand DNA oligonucleotide
acattttgctgtggaccttg

SEQ ID NO: 90 - DNA, 20 bp, Artificial sequence
  Single strand DNA oligonucleotide
gcctcgaagatgtttgctct

SEQ ID NO: 91 - DNA, 20 bp, Artificial sequence
  Single strand DNA oligonucleotide
cgggcataatgacctttttc

SEQ ID NO: 92 - DNA, 20 bp, Artificial sequence
  Single strand DNA oligonucleotide
agagcctcgaagatgtttgc

SEQ ID NO: 93 - DNA, 20 bp, Artificial sequence
  Single strand DNA oligonucleotide
ctgtaggccagtgagcgttt

SEQ ID NO: 94 - DNA, 20 bp, Artificial sequence
  Single strand DNA oligonucleotide
cgcagacctacaggaagcat

SEQ ID NO: 95 - DNA, 20 bp, Artificial sequence
  Single strand DNA oligonucleotide
gaggggaggcaaaagaaaac

SEQ ID NO: 96 - DNA, 20 bp, Artificial sequence
  Single strand DNA oligonucleotide
ccggaaaaattagcgttgaa

SEQ ID NO: 97 - DNA, 20 bp, Artificial sequence
  Single strand DNA oligonucleotide
caattctccggcatcaagtt

SEQ ID NO: 98 - DNA, 20 bp, Artificial sequence
  Single strand DNA oligonucleotide
tgtggttggactcatgcaat

SEQ ID NO: 99 - DNA, 20 bp, Artificial sequence
  Single strand DNA oligonucleotide
ttggcactaatcgcctaacc

SEQ ID NO: 100 - DNA, 20 bp, Artificial sequence
  Single strand DNA oligonucleotide
caaaaggagggcgataatga

SEQ ID NO: 101 - DNA, 20 bp, Artificial sequence
  Single strand DNA oligonucleotide
agcccaatgctgacttgaac

SEQ ID NO: 102 - DNA, 20 bp, Artificial sequence
  Single strand DNA oligonucleotide
cgggtgttgtcctaactgct

SEQ ID NO: 103 - DNA, 20 bp, Artificial sequence
  Single strand DNA oligonucleotide
gagtggtcattggcctcatt

SEQ ID NO: 104 - DNA, 20 bp, Artificial sequence
  Single strand DNA oligonucleotide
ataccgcccaatagggaaag

SEQ ID NO: 105 - DNA, 20 bp, Artificial sequence
  Single strand DNA oligonucleotide
gagtggtcattggcctcatt

SEQ ID NO: 106 - DNA, 20 bp, Artificial sequence
  Single strand DNA oligonucleotide
ataccgcccaatagggaaag

SEQ ID NO: 107 - DNA, 20 bp, Artificial sequence
  Single strand DNA oligonucleotide
tcattatcgccctccttttg

SEQ ID NO: 108 - DNA, 20 bp, Artificial sequence
  Single strand DNA oligonucleotide
ggggaaataacgggaaaaga

SEQ ID NO: 109 - DNA, 20 bp, Artificial sequence
  Single strand DNA oligonucleotide
gggcttgatcatttgtgctt

SEQ ID NO: 110 - DNA, 20 bp, Artificial sequence
  Single strand DNA oligonucleotide
gcgataactgggcaaatgat

SEQ ID NO: 111 - DNA, 20 bp, Artificial sequence
  Single strand DNA oligonucleotide
attgcatgagtccaaccaca

SEQ ID NO: 112 - DNA, 20 bp, Artificial sequence
  Single strand DNA oligonucleotide
tctttccgatggctcagtct

SEQ ID NO: 113 - DNA, 20 bp, Artificial sequence
  Single strand DNA oligonucleotide
cagacccgttttgcagagat

SEQ ID NO: 114 - DNA, 20 bp, Artificial sequence
  Single strand DNA oligonucleotide
gatggctcagtctggaaagg

SEQ ID NO: 115 - DNA, 20 bp, Artificial sequence
  Single strand DNA oligonucleotide
ttgaggctcccgtaacattc

SEQ ID NO: 116 - DNA, 20 bp, Artificial sequence
  Single strand DNA oligonucleotide
ggaatggtgctgtttccatt

SEQ ID NO: 117 - DNA, 20 bp, Artificial sequence
  Single strand DNA oligonucleotide
cagacccgttttgcagagat

SEQ ID NO: 118 - DNA, 20 bp, Artificial sequence
  Single strand DNA oligonucleotide
gcacgtgtcacactgaaaaa

SEQ ID NO: 119 - DNA, 20 bp, Artificial sequence
  Single strand DNA oligonucleotide
aaacagcaccattccacaca

SEQ ID NO: 120 - DNA, 19 bp, Artificial sequence
  Single strand DNA oligonucleotide
aaaactccagctgggatgg

SEQ ID NO: 121 - DNA, 20 bp, Artificial sequence
  Single strand DNA oligonucleotide
cgttaatggaaacagcacca

SEQ ID NO: 122 - DNA, 21 bp, Artificial sequence
  Single strand DNA oligonucleotide
cgtaggtaaaggtctggatgg

SEQ ID NO: 123 - DNA, 20 bp, Artificial sequence
  Single strand DNA oligonucleotide
gaaccgagcccattggtact

SEQ ID NO: 124 - DNA, 20 bp, Artificial sequence
  Single strand DNA oligonucleotide
gctgggatggtgatgtcaat

SEQ ID NO: 125 - DNA, 20 bp, Artificial sequence
  Single strand DNA oligonucleotide
gaaccgagcccattggtact

SEQ ID NO: 126 - DNA, 20 bp, Artificial sequence
  Single strand DNA oligonucleotide
tgctgtttttctgacggatg

SEQ ID NO: 127 - DNA, 20 bp, Artificial sequence
  Single strand DNA oligonucleotide
gaaccgagcccattggtact

SEQ ID NO: 128 - DNA, 19 bp, Artificial sequence
  Single strand DNA oligonucleotide
aaaactccagctgggatgg

SEQ ID NO: 129 - DNA, 20 bp, Artificial sequence
  Single strand DNA oligonucleotide
aaacagcaccattccacaca

SEQ ID NO: 130 - DNA, 20 bp, Artificial sequence
  Single strand DNA oligonucleotide
agagtccgtctcccatagcc

SEQ ID NO: 131 - DNA, 20 bp, Artificial sequence
  Single strand DNA oligonucleotide
acttgcccaggttcttgaaa

SEQ ID NO: 132 - DNA, 20 bp, Artificial sequence
  Single strand DNA oligonucleotide
gtccgtctcccatagcctta

SEQ ID NO: 133 - DNA, 20 bp, Artificial sequence
  Single strand DNA oligonucleotide
acttgcccaggttcttgaaa

SEQ ID NO: 134 - DNA, 20 bp, Artificial sequence
  Single strand DNA oligonucleotide
aagtgtgccattgcctttct

SEQ ID NO: 135 - DNA, 20 bp, Artificial sequence
  Single strand DNA oligonucleotide
tattaaggcgccacaactgg

SEQ ID NO: 136 - DNA, 20 bp, Artificial sequence
  Single strand DNA oligonucleotide
caactggtactgagcgttcg

SEQ ID NO: 137 - DNA, 20 bp, Artificial sequence
  Single strand DNA oligonucleotide
gaaaaagagcgggtgaacaa

SEQ ID NO: 138 - DNA, 20 bp, Artificial sequence
  Single strand DNA oligonucleotide
gttaatatcgcgcgtccatc

SEQ ID NO: 139 - DNA, 20 bp, Artificial sequence
  Single strand DNA oligonucleotide
ctatccttgacccgacgaac

SEQ ID NO: 140 - DNA, 20 bp, Artificial sequence
  Single strand DNA oligonucleotide
ccattgcctttctgcttgtt

SEQ ID NO: 141 - DNA, 20 bp, Artificial sequence
  Single strand DNA oligonucleotide
tattaaggcgccacaactgg

SEQ ID NO: 142 - DNA, 19 bp, Artificial sequence
  Single strand DNA oligonucleotide
ttgcttcatcatcgccatt

SEQ ID NO: 143 - DNA, 20 bp, Artificial sequence
  Single strand DNA oligonucleotide
cgcaattagtgaccagatcg

SEQ ID NO: 144 - DNA, 20 bp, Artificial sequence
  Single strand DNA oligonucleotide
caactggtactgagcgttcg

SEQ ID NO: 145 - DNA, 20 bp, Artificial sequence
  Single strand DNA oligonucleotide
acttgcccaggttcttgaaa

SEQ ID NO: 146 - DNA, 19 bp, Artificial sequence
  Single strand DNA oligonucleotide
atgggcatcacgcaagata

SEQ ID NO: 147 - DNA, 20 bp, Artificial sequence
  Single strand DNA oligonucleotide
aatcgtgtccagccattttg

SEQ ID NO: 148 - DNA, 20 bp, Artificial sequence
  Single strand DNA oligonucleotide
gttgtggagcagcttgaaca

SEQ ID NO: 149 - DNA, 20 bp, Artificial sequence
  Single strand DNA oligonucleotide
gatatcctgcggacgctcta

SEQ ID NO: 150 - DNA, 20 bp, Artificial sequence
  Single strand DNA oligonucleotide
gttgtggagcagcttgaaca

SEQ ID NO: 151 - DNA, 20 bp, Artificial sequence
  Single strand DNA oligonucleotide
gctctatccctgctgaatgc

SEQ ID NO: 152 - DNA, 20 bp, Artificial sequence
  Single strand DNA oligonucleotide
ccaactacccaccctgtgtc

SEQ ID NO: 153 - DNA, 20 bp, Artificial sequence
  Single strand DNA oligonucleotide
ccgaatcgttgactcgtatg

SEQ ID NO: 154 - DNA, 20 bp, Artificial sequence
  Single strand DNA oligonucleotide
ccaactacccaccctgtgtc

SEQ ID NO: 155 - DNA, 20 bp, Artificial sequence
  Single strand DNA oligonucleotide
caaccgctcgaacaccttag

SEQ ID NO: 156 - DNA, 20 bp, Artificial sequence
  Single strand DNA oligonucleotide
ccacggtatatccctcggta

SEQ ID NO: 157 - DNA, 20 bp, Artificial sequence
  Single strand DNA oligonucleotide
cttcgggattcatggttagc

SEQ ID NO: 158 - DNA, 20 bp, Artificial sequence
  Single strand DNA oligonucleotide
ggcagtctttctggcatagg

SEQ ID NO: 159 - DNA, 20 bp, Artificial sequence
  Single strand DNA oligonucleotide
attgggtgagcctgattgaa

SEQ ID NO: 160 - DNA, 20 bp, Artificial sequence
  Single strand DNA oligonucleotide
ggtgctagactctgggcttg

SEQ ID NO: 161 - DNA, 20 bp, Artificial sequence
  Single strand DNA oligonucleotide
gatttcccacgctgtcactt

SEQ ID NO: 162 - DNA, 20 bp, Artificial sequence
  Single strand DNA oligonucleotide
tgatcagtgattgcgtgaca

SEQ ID NO: 163 - DNA, 20 bp, Artificial sequence
  Single strand DNA oligonucleotide
aagcccagagtctagcacca

SEQ ID NO: 164 - DNA, 20 bp, Artificial sequence
  Single strand DNA oligonucleotide
ggtgctagactctgggcttg

SEQ ID NO: 165 - DNA, 20 bp, Artificial sequence
  Single strand DNA oligonucleotide
agcatacccaaaatggcaac

SEQ ID NO: 166 - DNA, 20 bp, Artificial sequence
  Single strand DNA oligonucleotide
aggtcgatctacgcgaaaaa

SEQ ID NO: 167 - DNA, 20 bp, Artificial sequence
  Single strand DNA oligonucleotide
gcaaagcctatgccagaaag

SEQ ID NO: 168 - DNA, 20 bp, Artificial sequence
  Single strand DNA oligonucleotide
atcagaacccgacgacaaag

SEQ ID NO: 169 - DNA, 20 bp, Artificial sequence
  Single strand DNA oligonucleotide
tccaggcttcgaggagagta

SEQ ID NO: 170 - DNA, 20 bp, Artificial sequence
  Single strand DNA oligonucleotide
aacaagtccagtgcatcacg

SEQ ID NO: 171 - DNA, 20 bp, Artificial sequence
  Single strand DNA oligonucleotide
gtcaggacgatttcgttggt

SEQ ID NO: 172 - DNA, 20 bp, Artificial sequence
  Single strand DNA oligonucleotide
caggtatggcaaggacgatt

SEQ ID NO: 173 - DNA, 20 bp, Artificial sequence
  Single strand DNA oligonucleotide
atgaacccttgcgaatcaag

SEQ ID NO: 174 - DNA, 20 bp, Artificial sequence
  Single strand DNA oligonucleotide
cttgattcgcaagggttcat

SEQ ID NO: 175 - DNA, 20 bp, Artificial sequence
  Single strand DNA oligonucleotide
ggagttacgcgagttgcttt

SEQ ID NO: 176 - DNA, 20 bp, Artificial sequence
  Single strand DNA oligonucleotide
ttccatgactcgtcagcaag

SEQ ID NO: 177 - DNA, 20 bp, Artificial sequence
  Single strand DNA oligonucleotide
atgtcccatatagcccgttg

SEQ ID NO: 178 - DNA, 20 bp, Artificial sequence
  Single strand DNA oligonucleotide
cttgattcgcaagggttcat

SEQ ID NO: 179 - DNA, 20 bp, Artificial sequence
  Single strand DNA oligonucleotide
ctttctcggacgaacgattt

SEQ ID NO: 180 - DNA, 20 bp, Artificial sequence
  Single strand DNA oligonucleotide
ttgccaacgatacaaatcca

SEQ ID NO: 181 - DNA, 20 bp, Artificial sequence
  Single strand DNA oligonucleotide
tcgaaattgacccgaaactc

SEQ ID NO: 182 - DNA, 20 bp, Artificial sequence
  Single strand DNA oligonucleotide
tttttggaattggcagcatc

SEQ ID NO: 183 - DNA, 20 bp, Artificial sequence
  Single strand DNA oligonucleotide
gcgttgtgttcttctgtgga

SEQ ID NO: 184 - DNA, 20 bp, Artificial sequence
  Single strand DNA oligonucleotide
gctttggcttaagggctttt

SEQ ID NO: 185 - DNA, 20 bp, Artificial sequence
  Single strand DNA oligonucleotide
aattgagcgtgaggttttcg

SEQ ID NO: 186 - DNA, 20 bp, Artificial sequence
  Single strand DNA oligonucleotide
ccacatgaaagagacggtca

SEQ ID NO: 187 - DNA, 20 bp, Artificial sequence
  Single strand DNA oligonucleotide
gggcgctattgatactcagg

SEQ ID NO: 188 - DNA, 20 bp, Artificial sequence
  Single strand DNA oligonucleotide
aagcctggcctgtacgttta

SEQ ID NO: 189 - DNA, 20 bp, Artificial sequence
  Single strand DNA oligonucleotide
acctggtttcaaagggttgg

SEQ ID NO: 190 - DNA, 20 bp, Artificial sequence
  Single strand DNA oligonucleotide
gcagggatccagctaattga

SEQ ID NO: 191 - DNA, 21 bp, Artificial sequence
  Single strand DNA oligonucleotide
aacagggtttgggacattttt

SEQ ID NO: 192 - DNA, 19 bp, Artificial sequence
  Single strand DNA oligonucleotide
cgattcgcaagaatctgga

SEQ ID NO: 193 - DNA, 20 bp, Artificial sequence
  Single strand DNA oligonucleotide
ttttgcctctgtcaccatca

SEQ ID NO: 194 - DNA, 20 bp, Artificial sequence
  Single strand DNA oligonucleotide
aaaatgcagggatccagcta

SEQ ID NO: 195 - DNA, 23 bp, Artificial sequence
  Single strand DNA oligonucleotide
gcggataagaaaagacaatagcc

SEQ ID NO: 196 - DNA, 25 bp, Artificial sequence
  Single strand DNA oligonucleotide
cgagctgataataaattatggaacc

SEQ ID NO: 197 - DNA, 20 bp, Artificial sequence
  Single strand DNA oligonucleotide
gcactgcagagccagagata

SEQ ID NO: 198 - DNA, 25 bp, Artificial sequence
  Single strand DNA oligonucleotide
cgagctgataataaattatggaacc

SEQ ID NO: 199 - DNA, 20 bp, Artificial sequence
  Single strand DNA oligonucleotide
atctctcgcgggtagttgag

SEQ ID NO: 200 - DNA, 20 bp, Artificial sequence
  Single strand DNA oligonucleotide
ttgatggtgacagaggcaaa

SEQ ID NO: 201 - DNA, 20 bp, Artificial sequence
  Single strand DNA oligonucleotide
atctctcgcgggtagttgag

SEQ ID NO: 202 - DNA, 20 bp, Artificial sequence
  Single strand DNA oligonucleotide
ctcgcttttcccaagtgaat

SEQ ID NO: 203 - DNA, 20 bp, Artificial sequence
  Single strand DNA oligonucleotide
gagatgagtttcgggagcag

SEQ ID NO: 204 - DNA, 20 bp, Artificial sequence
  Single strand DNA oligonucleotide
ggaaacaaaccgactggaaa

SEQ ID NO: 205 - DNA, 20 bp, Artificial sequence
  Single strand DNA oligonucleotide
caccccaccagaaccataaa

SEQ ID NO: 206 - DNA, 20 bp, Artificial sequence
  Single strand DNA oligonucleotide
gaatactgatgcggcagtcc

SEQ ID NO: 207 - DNA, 20 bp, Artificial sequence
  Single strand DNA oligonucleotide
tgggcaatgattgtttgtgt

SEQ ID NO: 208 - DNA, 20 bp, Artificial sequence
  Single strand DNA oligonucleotide
ggcttatgggaaagcactca

SEQ ID NO: 209 - DNA, 20 bp, Artificial sequence
  Single strand DNA oligonucleotide
tgggcaatgattgtttgtgt

SEQ ID NO: 210 - DNA, 20 bp, Artificial sequence
  Single strand DNA oligonucleotide
cgattgtggctgaaattgaa

SEQ ID NO: 211 - DNA, 20 bp, Artificial sequence
  Single strand DNA oligonucleotide
agctagatcgccactggaaa

SEQ ID NO: 212 - DNA, 20 bp, Artificial sequence
  Single strand DNA oligonucleotide
gcgacaacatcagaaaacga

SEQ ID NO: 213 - DNA, 20 bp, Artificial sequence
  Single strand DNA oligonucleotide
ttgctcatggcgaaataaca

SEQ ID NO: 214 - DNA, 20 bp, Artificial sequence
  Single strand DNA oligonucleotide
gcccactttttccagtcaaa

SEQ ID NO: 215 - DNA, 20 bp, Artificial sequence
  Single strand DNA oligonucleotide
gctagatcgccactggaaac

SEQ ID NO: 216 - DNA, 20 bp, Artificial sequence
  Single strand DNA oligonucleotide
aggtgcagaaatgagcgagt

SEQ ID NO: 217 - DNA, 20 bp, Artificial sequence
  Single strand DNA oligonucleotide
tttgccaatattccccagag

SEQ ID NO: 218 - DNA, 20 bp, Artificial sequence
  Single strand DNA oligonucleotide
gcgacaacatcagaaaacga

SEQ ID NO: 219 - DNA, 20 bp, Artificial sequence
  Single strand DNA oligonucleotide
agctagatcgccactggaaa

SEQ ID NO: 220 - DNA, 20 bp, Artificial sequence
  Single strand DNA oligonucleotide
aggtgcagaaatgagcgagt

SEQ ID NO: 221 - DNA, 21 bp, Artificial sequence
  Single strand DNA oligonucleotide
ggttttacgacatcctcatcg

SEQ ID NO: 222 - DNA, 20 bp, Artificial sequence
  Single strand DNA oligonucleotide
actttgggagaagtggctca

SEQ ID NO: 223 - DNA, 20 bp, Artificial sequence
  Single strand DNA oligonucleotide
ttggtcagggtgtcgaattt

SEQ ID NO: 224 - DNA, 20 bp, Artificial sequence
  Single strand DNA oligonucleotide
tgcaaaacagagcaggaaaa

SEQ ID NO: 225 - DNA, 20 bp, Artificial sequence
  Single strand DNA oligonucleotide
ggcggatattcccaatcaat

SEQ ID NO: 226 - DNA, 20 bp, Artificial sequence
  Single strand DNA oligonucleotide
gtggaacaaatggcgatgta

SEQ ID NO: 227 - DNA, 20 bp, Artificial sequence
  Single strand DNA oligonucleotide
cggcatttttgctcctttag

SEQ ID NO: 228 - DNA, 20 bp, Artificial sequence
  Single strand DNA oligonucleotide
tgcaaaacagagcaggaaaa

SEQ ID NO: 229 - DNA, 20 bp, Artificial sequence
  Single strand DNA oligonucleotide
caccgatacaatctgcttgc

SEQ ID NO: 230 - DNA, 20 bp, Artificial sequence
  Single strand DNA oligonucleotide
atctgctgcaaaacagagca

SEQ ID NO: 231 - DNA, 19 bp, Artificial sequence
  Single strand DNA oligonucleotide
gagtggcggtacagggatt

SEQ ID NO: 232 - DNA, 19 bp, Artificial sequence
  Single strand DNA oligonucleotide
gcagggcttggagatcatt

SEQ ID NO: 233 - DNA, 20 bp, Artificial sequence
  Single strand DNA oligonucleotide
gagggcgggatttctacttc

SEQ ID NO: 234 - DNA, 20 bp, Artificial sequence
  Single strand DNA oligonucleotide
agccaggcaaaaggatacaa

SEQ ID NO: 235 - DNA, 20 bp, Artificial sequence
  Single strand DNA oligonucleotide
catgccaatccatcactgaa

SEQ ID NO: 236 - DNA, 20 bp, Artificial sequence
  Single strand DNA oligonucleotide
cattcgtgcaagcaagagaa

SEQ ID NO: 237 - DNA, 20 bp, Artificial sequence
  Single strand DNA oligonucleotide
tttcaagacctgcaccttca

SEQ ID NO: 238 - DNA, 20 bp, Artificial sequence
  Single strand DNA oligonucleotide
aattatgggagcaaggcaga

SEQ ID NO: 239 - DNA, 20 bp, Artificial sequence
  Single strand DNA oligonucleotide
cctctaggatccggctcaat

SEQ ID NO: 240 - DNA, 20 bp, Artificial sequence
  Single strand DNA oligonucleotide
tgaaggtgcaggtcttgaaa

SEQ ID NO: 241 - DNA, 20 bp, Artificial sequence
  Single strand DNA oligonucleotide
ggattcctcagcgctaactg

SEQ ID NO: 242 - DNA, 20 bp, Artificial sequence
  Single strand DNA oligonucleotide
gcgcaacggataaggataaa

SEQ ID NO: 243 - DNA, 21 bp, Artificial sequence
  Single strand DNA oligonucleotide
ccgccatatcgttgttatacg

SEQ ID NO: 244 - DNA, 20 bp, Artificial sequence
  Single strand DNA oligonucleotide
ggcattaacccgtcttctga

SEQ ID NO: 245 - DNA, 20 bp, Artificial sequence
  Single strand DNA oligonucleotide
ctgttttcggaaatgcctgt

SEQ ID NO: 246 - DNA, 20 bp, Artificial sequence
  Single strand DNA oligonucleotide
gggcagactatcaggcagag

SEQ ID NO: 247 - DNA, 20 bp, Artificial sequence
  Single strand DNA oligonucleotide
tgctgtgcgtaattgtggat

SEQ ID NO: 248 - DNA, 20 bp, Artificial sequence
  Single strand DNA oligonucleotide
gggcagactatcaggcagag

SEQ ID NO: 249 - DNA, 20 bp, Artificial sequence
  Single strand DNA oligonucleotide
cgagtttgatacgcccttct

SEQ ID NO: 250 - DNA, 20 bp, Artificial sequence
  Single strand DNA oligonucleotide
ggaggttcagcaacaacgat

SEQ ID NO: 251 - DNA, 20 bp, Artificial sequence
  Single strand DNA oligonucleotide
gctcgtcaacacttccttcc

SEQ ID NO: 252 - DNA, 20 bp, Artificial sequence
  Single strand DNA oligonucleotide
caattacgcacagcaactgg

SEQ ID NO: 253 - DNA, 20 bp, Artificial sequence
  Single strand DNA oligonucleotide
cgagtttgatacgcccttct

SEQ ID NO: 254 - DNA, 19 bp, Artificial sequence
  Single strand DNA oligonucleotide
acgctccccctgtaaaaca

SEQ ID NO: 255 - DNA, 20 bp, Artificial sequence
  Single strand DNA oligonucleotide
ggatgtgatccatctggtga

SEQ ID NO: 256 - DNA, 20 bp, Artificial sequence
  Single strand DNA oligonucleotide
gaggcataaacccatgctgt

SEQ ID NO: 257 - DNA, 20 bp, Artificial sequence
  Single strand DNA oligonucleotide
atggatttgcctgtctgacc

SEQ ID NO: 258 - DNA, 20 bp, Artificial sequence
  Single strand DNA oligonucleotide
tggcataaccgatgaacaga

SEQ ID NO: 259 - DNA, 20 bp, Artificial sequence
  Single strand DNA oligonucleotide
agacaacgggctgtagcatt

SEQ ID NO: 260 - DNA, 20 bp, Artificial sequence
  Single strand DNA oligonucleotide
cgtacctggaggcataaacc

SEQ ID NO: 261 - DNA, 20 bp, Artificial sequence
  Single strand DNA oligonucleotide
ggatgtgatccatctggtga

SEQ ID NO: 262 - DNA, 24 bp, Artificial sequence
  Single strand DNA oligonucleotide
aacagttcctgagtgtaatcacca

SEQ ID NO: 263 - DNA, 20 bp, Artificial sequence
  Single strand DNA oligonucleotide
actaatgcgagtgcttgctg

SEQ ID NO: 264 - DNA, 20 bp, Artificial sequence
  Single strand DNA oligonucleotide
atgttccgattcgcaatgtt

SEQ ID NO: 265 - DNA, 20 bp, Artificial sequence
  Single strand DNA oligonucleotide
ccagcaatcgtttgtgtttg

SEQ ID NO: 266 - DNA, 20 bp, Artificial sequence
  Single strand DNA oligonucleotide
attcgcccgaacatacaaac

SEQ ID NO: 267 - DNA, 20 bp, Artificial sequence
  Single strand DNA oligonucleotide
gccaatatgactcacccaca

SEQ ID NO: 268 - DNA, 20 bp, Artificial sequence
  Single strand DNA oligonucleotide
aatatacccgctggtccaaa

SEQ ID NO: 269 - DNA, 20 bp, Artificial sequence
  Single strand DNA oligonucleotide
ccgggtatggaaatcacttg

SEQ ID NO: 270 - DNA, 20 bp, Artificial sequence
  Single strand DNA oligonucleotide
atgttccgattcgcaatgtt

SEQ ID NO: 271 - DNA, 20 bp, Artificial sequence
  Single strand DNA oligonucleotide
actaatgcgagtgcttgctg

SEQ ID NO: 272 - DNA, 20 bp, Artificial sequence
  Single strand DNA oligonucleotide
attcgcccgaacatacaaac

SEQ ID NO: 273 - DNA, 20 bp, Artificial sequence
  Single strand DNA oligonucleotide
actaatgcgagtgcttgctg

SEQ ID NO: 274 - DNA, 20 bp, Artificial sequence
  Single strand DNA oligonucleotide
gtactttcacctcccgacca

SEQ ID NO: 275 - DNA, 20 bp, Artificial sequence
  Single strand DNA oligonucleotide
caatgacatggacgctggta

SEQ ID NO: 276 - DNA, 20 bp, Artificial sequence
  Single strand DNA oligonucleotide
gcaccctatccattcaccat

SEQ ID NO: 277 - DNA, 20 bp, Artificial sequence
  Single strand DNA oligonucleotide
gcatcgccagcgttattatt

SEQ ID NO: 278 - DNA, 20 bp, Artificial sequence
  Single strand DNA oligonucleotide
tgccagtcagtgagctatgg

SEQ ID NO: 279 - DNA, 20 bp, Artificial sequence
  Single strand DNA oligonucleotide
aaagccattaaggcgtaggg

SEQ ID NO: 280 - DNA, 20 bp, Artificial sequence
  Single strand DNA oligonucleotide
aataataacgctggcgatgc

SEQ ID NO: 281 - DNA, 20 bp, Artificial sequence
  Single strand DNA oligonucleotide
tgccatagctcactgactgg

SEQ ID NO: 282 - DNA, 20 bp, Artificial sequence
  Single strand DNA oligonucleotide
ggtagcaccagtcaggctgt

SEQ ID NO: 283 - DNA, 20 bp, Artificial sequence
  Single strand DNA oligonucleotide
cggaattgaaaacctctgct

SEQ ID NO: 284 - DNA, 20 bp, Artificial sequence
  Single strand DNA oligonucleotide
tgaaagttcgttcagcttgc

SEQ ID NO: 285 - DNA, 20 bp, Artificial sequence
  Single strand DNA oligonucleotide
gcatcgccagcgttattatt

SEQ ID NO: 286 - DNA, 20 bp, Artificial sequence
  Single strand DNA oligonucleotide
ggtagcaccagtcaggctgt

SEQ ID NO: 287 - DNA, 21 bp, Artificial sequence
  Single strand DNA oligonucleotide
gcccagtatgatgtccagaaa

SEQ ID NO: 288 - DNA, 21 bp, Artificial sequence
  Single strand DNA oligonucleotide
catcctggaacagactgggta

SEQ ID NO: 289 - DNA, 20 bp, Artificial sequence
  Single strand DNA oligonucleotide
ataaaaccaatcggcccaac

SEQ ID NO: 290 - DNA, 20 bp, Artificial sequence
  Single strand DNA oligonucleotide
catggcaacttacggcatta

SEQ ID NO: 291 - DNA, 20 bp, Artificial sequence
  Single strand DNA oligonucleotide
cgtaacagggaggaaagacg

SEQ ID NO: 292 - DNA, 20 bp, Artificial sequence
  Single strand DNA oligonucleotide
acgcatgggagaagaaagag

SEQ ID NO: 293 - DNA, 20 bp, Artificial sequence
  Single strand DNA oligonucleotide
cgataatgctgcaagcaaac

SEQ ID NO: 294 - DNA, 20 bp, Artificial sequence
  Single strand DNA oligonucleotide
acagcaactgcgtctttcct

SEQ ID NO: 295 - DNA, 20 bp, Artificial sequence
  Single strand DNA oligonucleotide
ataaaaccaatcggcccaac

SEQ ID NO: 296 - DNA, 20 bp, Artificial sequence
  Single strand DNA oligonucleotide
ccaataggcatcgtcacctc

SEQ ID NO: 297 - DNA, 21 bp, Artificial sequence
  Single strand DNA oligonucleotide
gccagatgttctatccccagt

SEQ ID NO: 298 - DNA, 20 bp, Artificial sequence
  Single strand DNA oligonucleotide
aatggttgcggtaaatcgac

SEQ ID NO: 299 - DNA, 20 bp, Artificial sequence
  Single strand DNA oligonucleotide
tggcatttgcattaggttga

SEQ ID NO: 300 - DNA, 20 bp, Artificial sequence
  Single strand DNA oligonucleotide
attctgggaccaaatggttg

SEQ ID NO: 301 - DNA, 20 bp, Artificial sequence
  Single strand DNA oligonucleotide
tggcatttgcattaggttga

SEQ ID NO: 302 - DNA, 20 bp, Artificial sequence
  Single strand DNA oligonucleotide
tacctgaactgcaacgagga

SEQ ID NO: 303 - DNA, 20 bp, Artificial sequence
  Single strand DNA oligonucleotide
aatgtcgatttaccgcaacc

SEQ ID NO: 304 - DNA, 20 bp, Artificial sequence
  Single strand DNA oligonucleotide
cgctatcgcaggagtttgtt

SEQ ID NO: 305 - DNA, 20 bp, Artificial sequence
  Single strand DNA oligonucleotide
cgcgtagggaaaccagaata

SEQ ID NO: 306 - DNA, 20 bp, Artificial sequence
  Single strand DNA oligonucleotide
cgctatcgcaggagtttgtt

SEQ ID NO: 307 - DNA, 18 bp, Artificial sequence
  Single strand DNA oligonucleotide
atctttgacgcgcgtagg

SEQ ID NO: 308 - DNA, 20 bp, Artificial sequence
  Single strand DNA oligonucleotide
cgctatcgcaggagtttgtt

SEQ ID NO: 309 - DNA, 18 bp, Artificial sequence
  Single strand DNA oligonucleotide
atgtttcccgcccatctt

SEQ ID NO: 310 - DNA, 23 bp, Artificial sequence
  Single strand DNA oligonucleotide
ccatcagttccatgttatggatt

SEQ ID NO: 311 - DNA, 20 bp, Artificial sequence
  Single strand DNA oligonucleotide
ctctgatgtatccggtgtgc

SEQ ID NO: 312 - DNA, 20 bp, Artificial sequence
  Single strand DNA oligonucleotide
tacaatgaatgccctgttgg

SEQ ID NO: 313 - DNA, 20 bp, Artificial sequence
  Single strand DNA oligonucleotide
atggccagtgagcgtaaaaa

SEQ ID NO: 314 - DNA, 23 bp, Artificial sequence
  Single strand DNA oligonucleotide
ccatcagttccatgttatggatt

SEQ ID NO: 315 - DNA, 19 bp, Artificial sequence
  Single strand DNA oligonucleotide
ggtgcattgattccacgtc

SEQ ID NO: 316 - DNA, 20 bp, Artificial sequence
  Single strand DNA oligonucleotide
ccgtggacgaatagagcatt

SEQ ID NO: 317 - DNA, 20 bp, Artificial sequence
  Single strand DNA oligonucleotide
tgagaagttccgggagagaa

SEQ ID NO: 318 - DNA, 20 bp, Artificial sequence
  Single strand DNA oligonucleotide
agttgctgctgacgaccttc

SEQ ID NO: 319 - DNA, 20 bp, Artificial sequence
  Single strand DNA oligonucleotide
tttttggtgcattgattcca

SEQ ID NO: 320 - DNA, 20 bp, Artificial sequence
  Single strand DNA oligonucleotide
ctgtggttgaggtttgagca

SEQ ID NO: 321 - DNA, 20 bp, Artificial sequence
  Single strand DNA oligonucleotide
atggccagtgagcgtaaaaa

SEQ ID NO: 322 - DNA, 20 bp, Artificial sequence
  Single strand DNA oligonucleotide
cctctagttgctgctgacga

SEQ ID NO: 323 - DNA, 20 bp, Artificial sequence
  Single strand DNA oligonucleotide
tttttggtgcattgattcca

SEQ ID NO: 324 - DNA, 20 bp, Artificial sequence
  Single strand DNA oligonucleotide
cggtttcgatacgctctttt

SEQ ID NO: 325 - DNA, 20 bp, Artificial sequence
  Single strand DNA oligonucleotide
atgggttttggatctgaacg

SEQ ID NO: 326 - DNA, 20 bp, Artificial sequence
  Single strand DNA oligonucleotide
ggtctcggaagatcgagaaa

SEQ ID NO: 327 - DNA, 20 bp, Artificial sequence
  Single strand DNA oligonucleotide
ccgggcaaaagtatcaaaaa

SEQ ID NO: 328 - DNA, 20 bp, Artificial sequence
  Single strand DNA oligonucleotide
tggaatcaatgcaccaaaaa

SEQ ID NO: 329 - DNA, 20 bp, Artificial sequence
  Single strand DNA oligonucleotide
ccgggcaaaagtatcaaaaa

SEQ ID NO: 330 - DNA, 20 bp, Artificial sequence
  Single strand DNA oligonucleotide
ttctctcccggaacttctca

SEQ ID NO: 331 - DNA, 20 bp, Artificial sequence
  Single strand DNA oligonucleotide
aaatacggggaattgtgtgg

SEQ ID NO: 332 - DNA, 20 bp, Artificial sequence
  Single strand DNA oligonucleotide
gtggaatcaatgcaccaaaa

SEQ ID NO: 333 - DNA, 20 bp, Artificial sequence
  Single strand DNA oligonucleotide
ccgggcaaaagtatcaaaaa

SEQ ID NO: 334 - DNA, 20 bp, Artificial sequence
  Single strand DNA oligonucleotide
ccgtaacggtcaaaaatcgt

SEQ ID NO: 335 - DNA, 20 bp, Artificial sequence
  Single strand DNA oligonucleotide
ttttgttcaccacgacctca

SEQ ID NO: 336 - DNA, 20 bp, Artificial sequence
  Single strand DNA oligonucleotide
aaaaatcgtggcgttgacac

SEQ ID NO: 337 - DNA, 20 bp, Artificial sequence
  Single strand DNA oligonucleotide
ttttgttcaccacgacctca

SEQ ID NO: 338 - DNA, 20 bp, Artificial sequence
  Single strand DNA oligonucleotide
gtatcgactggtggcatctg

SEQ ID NO: 339 - DNA, 20 bp, Artificial sequence
  Single strand DNA oligonucleotide
tgactgcctttctcccactt

SEQ ID NO: 340 - DNA, 20 bp, Artificial sequence
  Single strand DNA oligonucleotide
ggcatctggagatacgcttt

SEQ ID NO: 341 - DNA, 20 bp, Artificial sequence
  Single strand DNA oligonucleotide
tgactgcctttctcccactt

SEQ ID NO: 342 - DNA, 21 bp, Artificial sequence
  Single strand DNA oligonucleotide
tgctttttgatgatggaaaca

SEQ ID NO: 343 - DNA, 20 bp, Artificial sequence
  Single strand DNA oligonucleotide
caaacggggaatgtagcaat

SEQ ID NO: 344 - DNA, 21 bp, Artificial sequence
  Single strand DNA oligonucleotide
tgcattgcggttttaatcttt

SEQ ID NO: 345 - DNA, 21 bp, Artificial sequence
  Single strand DNA oligonucleotide
agccaagccaatttatttcaa

SEQ ID NO: 346 - DNA, 20 bp, Artificial sequence
  Single strand DNA oligonucleotide
gtatcgactggtggcatctg

SEQ ID NO: 347 - DNA, 20 bp, Artificial sequence
  Single strand DNA oligonucleotide
cagcatgactgcctttctcc

SEQ ID NO: 348 - DNA, 22 bp, Artificial sequence
  Single strand DNA oligonucleotide
aatgcattgcggttttaatctt

SEQ ID NO: 349 - DNA, 20 bp, Artificial sequence
  Single strand DNA oligonucleotide
ttgaatagccaagccaattt

SEQ ID NO: 350 - DNA, 20 bp, Artificial sequence
  Single strand DNA oligonucleotide
ctgcagcattgacgatttgt

SEQ ID NO: 351 - DNA, 20 bp, Artificial sequence
  Single strand DNA oligonucleotide
caaacggggaatgtagcaat

SEQ ID NO: 352 - DNA, 20 bp, Artificial sequence
  Single strand DNA oligonucleotide
tgcagcattgacgatttgtt

SEQ ID NO: 353 - DNA, 19 bp, Artificial sequence
  Single strand DNA oligonucleotide
tatcacccaaacggggaat

SEQ ID NO: 354 - DNA, 25 bp, Artificial sequence
  Single strand DNA oligonucleotide
cggacataaatatctcaaaatgaca

SEQ ID NO: 355 - DNA, 20 bp, Artificial sequence
  Single strand DNA oligonucleotide
caaacggggaatgtagcaat

SEQ ID NO: 356 - DNA, 20 bp, Artificial sequence
  Single strand DNA oligonucleotide
gagcgtctgctcaaacaggt

SEQ ID NO: 357 - DNA, 19 bp, Artificial sequence
  Single strand DNA oligonucleotide
gtagtgggtgaggggctgt

SEQ ID NO: 358 - DNA, 21 bp, Artificial sequence
  Single strand DNA oligonucleotide
tcaaacaggttatccgtcagg

SEQ ID NO: 359 - DNA, 19 bp, Artificial sequence
  Single strand DNA oligonucleotide
gtagtgggtgaggggctgt

SEQ ID NO: 360 - DNA, 20 bp, Artificial sequence
  Single strand DNA oligonucleotide
tccgtcaggaagaggaaaaa

SEQ ID NO: 361 - DNA, 20 bp, Artificial sequence
  Single strand DNA oligonucleotide
cagagcgtcaagcaaccttt

SEQ ID NO: 362 - DNA, 20 bp, Artificial sequence
  Single strand DNA oligonucleotide
tccgtcaggaagaggaaaaa

SEQ ID NO: 363 - DNA, 20 bp, Artificial sequence
  Single strand DNA oligonucleotide
ccttacctatttccgctggt

SEQ ID NO: 364 - DNA, 20 bp, Artificial sequence
  Single strand DNA oligonucleotide
ttgaaaaccagagccttgct

SEQ ID NO: 365 - DNA, 22 bp, Artificial sequence
  Single strand DNA oligonucleotide
cgttgaccataagaaaacctca

SEQ ID NO: 366 - DNA, 21 bp, Artificial sequence
  Single strand DNA oligonucleotide
aatgaaaaaggaaacgccata

SEQ ID NO: 367 - DNA, 22 bp, Artificial sequence
  Single strand DNA oligonucleotide
cgttgaccataagaaaacctca

SEQ ID NO: 368 - DNA, 20 bp, Artificial sequence
  Single strand DNA oligonucleotide
ttttctgctgcaagcacttc

SEQ ID NO: 369 - DNA, 20 bp, Artificial sequence
  Single strand DNA oligonucleotide
ggcccgtttatcagaaaggt

SEQ ID NO: 370 - DNA, 21 bp, Artificial sequence
  Single strand DNA oligonucleotide
aaaagggaaggccttatgatg

SEQ ID NO: 371 - DNA, 21 bp, Artificial sequence
  Single strand DNA oligonucleotide
tccaaaatgggaaagaagagg

SEQ ID NO: 372 - DNA, 22 bp, Artificial sequence
  Single strand DNA oligonucleotide
tttcataggaagtggaggtggt

SEQ ID NO: 373 - DNA, 20 bp, Artificial sequence
  Single strand DNA oligonucleotide
aaaatggcccgtttatcaga

SEQ ID NO: 374 - DNA, 20 bp, Artificial sequence
  Single strand DNA oligonucleotide
ttttctgctgcaagcacttc

SEQ ID NO: 375 - DNA, 20 bp, Artificial sequence
  Single strand DNA oligonucleotide
ccaaaatgggaaagaagagg

SEQ ID NO: 376 - DNA, 21 bp, Artificial sequence
  Single strand DNA oligonucleotide
ccttgcttagacctgtgtcca

SEQ ID NO: 377 - DNA, 20 bp, Artificial sequence
  Single strand DNA oligonucleotide
ggcccgtttatcagaaaggt

SEQ ID NO: 378 - DNA, 20 bp, Artificial sequence
  Single strand DNA oligonucleotide
acgtgggttggatcattgtt

SEQ ID NO: 379 - DNA, 20 bp, Artificial sequence
  Single strand DNA oligonucleotide
aaaatggcccgtttatcaga

SEQ ID NO: 380 - DNA, 21 bp, Artificial sequence
  Single strand DNA oligonucleotide
gggtctcgatttgatgattga

SEQ ID NO: 381 - DNA, 22 bp, Artificial sequence
  Single strand DNA oligonucleotide
ggctttaccgtggtagtactgg

SEQ ID NO: 382 - DNA, 20 bp, Artificial sequence
  Single strand DNA oligonucleotide
tcttggtagggacgtggttt

SEQ ID NO: 383 - DNA, 22 bp, Artificial sequence
  Single strand DNA oligonucleotide
ggctttaccgtggtagtactgg

SEQ ID NO: 384 - DNA, 20 bp, Artificial sequence
  Single strand DNA oligonucleotide
cttcattttggcctctttgc

SEQ ID NO: 385 - DNA, 20 bp, Artificial sequence
  Single strand DNA oligonucleotide
ttgggtagccacatccctta

SEQ ID NO: 386 - DNA, 20 bp, Artificial sequence
  Single strand DNA oligonucleotide
gatgtggctacccaagcaat

SEQ ID NO: 387 - DNA, 20 bp, Artificial sequence
  Single strand DNA oligonucleotide
cacccaaggactgaaggaag

SEQ ID NO: 388 - DNA, 20 bp, Artificial sequence
  Single strand DNA oligonucleotide
gggcatgggcatacttatca

SEQ ID NO: 389 - DNA, 20 bp, Artificial sequence
  Single strand DNA oligonucleotide
atttcccctcaattccttcg

SEQ ID NO: 390 - DNA, 20 bp, Artificial sequence
  Single strand DNA oligonucleotide
tgtgcaccttagtcgctttg

SEQ ID NO: 391 - DNA, 20 bp, Artificial sequence
  Single strand DNA oligonucleotide
atttcccctcaattccttcg

SEQ ID NO: 392 - DNA, 20 bp, Artificial sequence
  Single strand DNA oligonucleotide
cttcattttggcctctttgc

SEQ ID NO: 393 - DNA, 21 bp, Artificial sequence
  Single strand DNA oligonucleotide
tctgagcgatttttcttgagc

SEQ ID NO: 394 - DNA, 21 bp, Artificial sequence
  Single strand DNA oligonucleotide
atggcggtagctcataccttt

SEQ ID NO: 395 - DNA, 20 bp, Artificial sequence
  Single strand DNA oligonucleotide
atttcccctcaattccttcg

SEQ ID NO: 396 - DNA, 22 bp, Artificial sequence
  Single strand DNA oligonucleotide
cgccccatagtatattggacat

SEQ ID NO: 397 - DNA, 20 bp, Artificial sequence
  Single strand DNA oligonucleotide
acgatatcagcgaaggtgct

SEQ ID NO: 398 - DNA, 19 bp, Artificial sequence
  Single strand DNA oligonucleotide
tccaggtcgccccatagta

SEQ ID NO: 399 - DNA, 20 bp, Artificial sequence
  Single strand DNA oligonucleotide
gctgcactggtatccgattt

SEQ ID NO: 400 - DNA, 20 bp, Artificial sequence
  Single strand DNA oligonucleotide
ggggctcctgattcataacc

SEQ ID NO: 401 - DNA, 20 bp, Artificial sequence
  Single strand DNA oligonucleotide
acgatatcagcgaaggtgct

SEQ ID NO: 402 - DNA, 20 bp, Artificial sequence
  Single strand DNA oligonucleotide
caagccaaatgctgacaaaa

SEQ ID NO: 403 - DNA, 20 bp, Artificial sequence
  Single strand DNA oligonucleotide
attaagaaccgacgccagtg

SEQ ID NO: 404 - DNA, 20 bp, Artificial sequence
  Single strand DNA oligonucleotide
cggttctggctcaggtagtt

SEQ ID NO: 405 - DNA, 20 bp, Artificial sequence
  Single strand DNA oligonucleotide
tgaatcccacagcgtcatta

SEQ ID NO: 406 - DNA, 20 bp, Artificial sequence
  Single strand DNA oligonucleotide
ttatttagagccgcgctgac

SEQ ID NO: 407 - DNA, 20 bp, Artificial sequence
  Single strand DNA oligonucleotide
gcgaagatcctctggtaacg

SEQ ID NO: 408 - DNA, 20 bp, Artificial sequence
  Single strand DNA oligonucleotide
agcatcccccttgttattga

SEQ ID NO: 409 - DNA, 20 bp, Artificial sequence
  Single strand DNA oligonucleotide
gcaccagcagtacatcgaga

SEQ ID NO: 410 - DNA, 20 bp, Artificial sequence
  Single strand DNA oligonucleotide
atacccgctttctcaagtgc

SEQ ID NO: 411 - DNA, 20 bp, Artificial sequence
  Single strand DNA oligonucleotide
gctacgtgctggggtatctc

SEQ ID NO: 412 - DNA, 20 bp, Artificial sequence
  Single strand DNA oligonucleotide
cgggccatacatccagtaat

SEQ ID NO: 413 - DNA, 20 bp, Artificial sequence
  Single strand DNA oligonucleotide
gtgttcggcttgcagctatc

SEQ ID NO: 414 - DNA, 20 bp, Artificial sequence
  Single strand DNA oligonucleotide
ttctttagcttcgggccata

SEQ ID NO: 415 - DNA, 19 bp, Artificial sequence
  Single strand DNA oligonucleotide
ctatcgggggcgtagagaa

SEQ ID NO: 416 - DNA, 20 bp, Artificial sequence
  Single strand DNA oligonucleotide
taatgacgctgtgggattca

SEQ ID NO: 417 - DNA, 20 bp, Artificial sequence
  Single strand DNA oligonucleotide
gatgtatggcccgaagctaa

SEQ ID NO: 418 - DNA, 20 bp, Artificial sequence
  Single strand DNA oligonucleotide
gtcaagccaaatgctgacaa

SEQ ID NO: 419 - DNA, 20 bp, Artificial sequence
  Single strand DNA oligonucleotide
gcaccagcagtacatcgaga

SEQ ID NO: 420 - DNA, 20 bp, Artificial sequence
  Single strand DNA oligonucleotide
cgggcaaagactacacacag

SEQ ID NO: 421 - DNA, 24 bp, Artificial sequence
  Single strand DNA oligonucleotide
cagatcataggtgtaatgatcgaa

SEQ ID NO: 422 - DNA, 20 bp, Artificial sequence
  Single strand DNA oligonucleotide
gtatgcaggaaagcaccaca

SEQ ID NO: 423 - DNA, 20 bp, Artificial sequence
  Single strand DNA oligonucleotide
aaaccgctcaaaggtgaatg

SEQ ID NO: 424 - DNA, 20 bp, Artificial sequence
  Single strand DNA oligonucleotide
atacgttttcacgccgtttc

SEQ ID NO: 425 - DNA, 20 bp, Artificial sequence
  Single strand DNA oligonucleotide
cggcaatacgggtcagtaag

SEQ ID NO: 426 - DNA, 20 bp, Artificial sequence
  Single strand DNA oligonucleotide
atcgttttggctttggtgtc

SEQ ID NO: 427 - DNA, 20 bp, Artificial sequence
  Single strand DNA oligonucleotide
cgtaaatatcgggaggcgta

SEQ ID NO: 428 - DNA, 20 bp, Artificial sequence
  Single strand DNA oligonucleotide
tggtattgtgcgtacgtggt

SEQ ID NO: 429 - DNA, 20 bp, Artificial sequence
  Single strand DNA oligonucleotide
cgtaaatatcgggaggcgta

SEQ ID NO: 430 - DNA, 20 bp, Artificial sequence
  Single strand DNA oligonucleotide
ccctggtgcaggacatagat

SEQ ID NO: 431 - DNA, 20 bp, Artificial sequence
  Single strand DNA oligonucleotide
agctccccatggtttgctat

SEQ ID NO: 432 - DNA, 20 bp, Artificial sequence
  Single strand DNA oligonucleotide
gacaacaaactcccctggtg

SEQ ID NO: 433 - DNA, 20 bp, Artificial sequence
  Single strand DNA oligonucleotide
ttgctatcggacatgggtta

SEQ ID NO: 434 - DNA, 20 bp, Artificial sequence
  Single strand DNA oligonucleotide
ctggttcgtccactttcgat

SEQ ID NO: 435 - DNA, 20 bp, Artificial sequence
  Single strand DNA oligonucleotide
gcacgtcgtttggaattagg

SEQ ID NO: 436 - DNA, 20 bp, Artificial sequence
  Single strand DNA oligonucleotide
agctctcctgcctgaacgta

SEQ ID NO: 437 - DNA, 20 bp, Artificial sequence
  Single strand DNA oligonucleotide
agcccagactggctactgaa

SEQ ID NO: 438 - DNA, 20 bp, Artificial sequence
  Single strand DNA oligonucleotide
cgacccgagtaaaagaacga

SEQ ID NO: 439 - DNA, 20 bp, Artificial sequence
  Single strand DNA oligonucleotide
gccgagcaataacaccactt

SEQ ID NO: 440 - DNA, 20 bp, Artificial sequence
  Single strand DNA oligonucleotide
agccagatccagaagattgc

SEQ ID NO: 441 - DNA, 20 bp, Artificial sequence
  Single strand DNA oligonucleotide
gccgagcaataacaccactt

SEQ ID NO: 442 - DNA, 20 bp, Artificial sequence
  Single strand DNA oligonucleotide
agctctcctgcctgaacgta

SEQ ID NO: 443 - DNA, 20 bp, Artificial sequence
  Single strand DNA oligonucleotide
ggtcgctaaggtcattgctt

SEQ ID NO: 444 - DNA, 20 bp, Artificial sequence
  Single strand DNA oligonucleotide
tcgatacgtaatgcgaagca

SEQ ID NO: 445 - DNA, 20 bp, Artificial sequence
  Single strand DNA oligonucleotide
ttttactcgggtcgctaagg

SEQ ID NO: 446 - DNA, 20 bp, Artificial sequence
  Single strand DNA oligonucleotide
ggcgtggtacatggatatga

SEQ ID NO: 447 - DNA, 20 bp, Artificial sequence
  Single strand DNA oligonucleotide
gggcgacgaatgtattcaaa

SEQ ID NO: 448 - DNA, 20 bp, Artificial sequence
  Single strand DNA oligonucleotide
ccaggcgtggtacatggata

SEQ ID NO: 449 - DNA, 20 bp, Artificial sequence
  Single strand DNA oligonucleotide
gggcgacgaatgtattcaaa

SEQ ID NO: 450 - DNA, 20 bp, Artificial sequence
  Single strand DNA oligonucleotide
gcggtggctacactatggtt

SEQ ID NO: 451 - DNA, 20 bp, Artificial sequence
  Single strand DNA oligonucleotide
attggtacccagttcggtga

SEQ ID NO: 452 - DNA, 20 bp, Artificial sequence
  Single strand DNA oligonucleotide
atggcttcacggttaaatgc

SEQ ID NO: 453 - DNA, 20 bp, Artificial sequence
  Single strand DNA oligonucleotide
tggtacccagttcggtgatt

SEQ ID NO: 454 - DNA, 20 bp, Artificial sequence
  Single strand DNA oligonucleotide
ggcggatctgtatacgcaat

SEQ ID NO: 455 - DNA, 20 bp, Artificial sequence
  Single strand DNA oligonucleotide
acgcagaaaaggcagctaac

SEQ ID NO: 456 - DNA, 20 bp, Artificial sequence
  Single strand DNA oligonucleotide
tgtatacgcaatcggctttg

SEQ ID NO: 457 - DNA, 20 bp, Artificial sequence
  Single strand DNA oligonucleotide
tggtacccagttcggtgatt

SEQ ID NO: 458 - DNA, 20 bp, Artificial sequence
  Single strand DNA oligonucleotide
tcatcggatctttcccttgt

SEQ ID NO: 459 - DNA, 20 bp, Artificial sequence
  Single strand DNA oligonucleotide
agcgggtttgaaaggatgta

SEQ ID NO: 460 - DNA, 20 bp, Artificial sequence
  Single strand DNA oligonucleotide
agttgccaaccttgatcctg

SEQ ID NO: 461 - DNA, 20 bp, Artificial sequence
  Single strand DNA oligonucleotide
aagcgggtttgaaaggatgt

SEQ ID NO: 462 - DNA, 20 bp, Artificial sequence
  Single strand DNA oligonucleotide
tcatcggatctttcccttgt

SEQ ID NO: 463 - DNA, 20 bp, Artificial sequence
  Single strand DNA oligonucleotide
gaatcaggaacggctttttg

SEQ ID NO: 464 - DNA, 20 bp, Artificial sequence
  Single strand DNA oligonucleotide
cagttgccaaccttgatcct

SEQ ID NO: 465 - DNA, 20 bp, Artificial sequence
  Single strand DNA oligonucleotide
cctgctgaaacatggcaata

SEQ ID NO: 466 - DNA, 20 bp, Artificial sequence
  Single strand DNA oligonucleotide
accgaaaaagagcaagagca

SEQ ID NO: 467 - DNA, 20 bp, Artificial sequence
  Single strand DNA oligonucleotide
caatgcaaaatcccatccat

SEQ ID NO: 468 - DNA, 20 bp, Artificial sequence
  Single strand DNA oligonucleotide
attcggatggtatcgacgaa

SEQ ID NO: 469 - DNA, 20 bp, Artificial sequence
  Single strand DNA oligonucleotide
aggatcaaggttggcaactg

SEQ ID NO: 470 - DNA, 20 bp, Artificial sequence
  Single strand DNA oligonucleotide
tgccggtagtgttatggaca

SEQ ID NO: 471 - DNA, 20 bp, Artificial sequence
  Single strand DNA oligonucleotide
ccctgaaaagactcctgctg

SEQ ID NO: 472 - DNA, 20 bp, Artificial sequence
  Single strand DNA oligonucleotide
aaggtaaactctgcgctcca

SEQ ID NO: 473 - DNA, 20 bp, Artificial sequence
  Single strand DNA oligonucleotide
aggatcaaggttggcaactg

SEQ ID NO: 474 - DNA, 20 bp, Artificial sequence
  Single strand DNA oligonucleotide
tgccggtagtgttatggaca

SEQ ID NO: 475 - DNA, 20 bp, Artificial sequence
  Single strand DNA oligonucleotide
tccctgaaaagactcctgct

SEQ ID NO: 476 - DNA, 20 bp, Artificial sequence
  Single strand DNA oligonucleotide
gctgccggtagtgttatgga

SEQ ID NO: 477 - DNA, 20 bp, Artificial sequence
  Single strand DNA oligonucleotide
ccctgaaaagactcctgctg

SEQ ID NO: 478 - DNA, 20 bp, Artificial sequence
  Single strand DNA oligonucleotide
aaacaaagcgtcgcaaaaag

SEQ ID NO: 479 - DNA, 20 bp, Artificial sequence
  Single strand DNA oligonucleotide
caatgcaaaatcccatccat

SEQ ID NO: 480 - DNA, 20 bp, Artificial sequence
  Single strand DNA oligonucleotide
tacggtgaaagagtggcatt

SEQ ID NO: 481 - DNA, 20 bp, Artificial sequence
  Single strand DNA oligonucleotide
ctgcaacaatggctttttgt

SEQ ID NO: 482 - DNA, 22 bp, Artificial sequence
  Single strand DNA oligonucleotide
tgtctgcataattggctttacc

SEQ ID NO: 483 - DNA, 19 bp, Artificial sequence
  Single strand DNA oligonucleotide
ctcgttccctgcaacaatg

SEQ ID NO: 484 - DNA, 20 bp, Artificial sequence
  Single strand DNA oligonucleotide
acggtgaaagagtggcattg

SEQ ID NO: 485 - DNA, 21 bp, Artificial sequence
  Single strand DNA oligonucleotide
tgggctttctgtaggttttga

SEQ ID NO: 486 - DNA, 20 bp, Artificial sequence
  Single strand DNA oligonucleotide
ggctacttctccccaccatt

SEQ ID NO: 487 - DNA, 20 bp, Artificial sequence
  Single strand DNA oligonucleotide
gggacgttacgcaaatttct

SEQ ID NO: 488 - DNA, 20 bp, Artificial sequence
  Single strand DNA oligonucleotide
ttctcgtcaggcatttttcc

SEQ ID NO: 489 - DNA, 21 bp, Artificial sequence
  Single strand DNA oligonucleotide
tgggctttctgtaggttttga

SEQ ID NO: 490 - DNA, 20 bp, Artificial sequence
  Single strand DNA oligonucleotide
tgatcaaaccagccatcaac

SEQ ID NO: 491 - DNA, 20 bp, Artificial sequence
  Single strand DNA oligonucleotide
ctgcaacaatggctttttgt

SEQ ID NO: 492 - DNA, 20 bp, Artificial sequence
  Single strand DNA oligonucleotide
tcgtcaggcatttttccttt

SEQ ID NO: 493 - DNA, 21 bp, Artificial sequence
  Single strand DNA oligonucleotide
tgggctttctgtaggttttga

SEQ ID NO: 494 - DNA, 20 bp, Artificial sequence
  Single strand DNA oligonucleotide
ggctacttctccccaccatt

SEQ ID NO: 495 - DNA, 20 bp, Artificial sequence
  Single strand DNA oligonucleotide
gggcaacagccataggtaaa

SEQ ID NO: 496 - DNA, 20 bp, Artificial sequence
  Single strand DNA oligonucleotide
tctcgtcaggcatttttcct

SEQ ID NO: 497 - DNA, 20 bp, Artificial sequence
  Single strand DNA oligonucleotide
tcagttgatggctggtttga

SEQ ID NO: 498 - DNA, 26 bp, Artificial sequence
  Single strand DNA oligonucleotide
tgaaaaataacttaaataagggatgg

SEQ ID NO: 499 - DNA, 26 bp, Artificial sequence
  Single strand DNA oligonucleotide
gcgaattatttagtacaaaaagcgta

SEQ ID NO: 500 - DNA, 25 bp, Artificial sequence
  Single strand DNA oligonucleotide
tttcattatgctatttaagatgtga

SEQ ID NO: 501 - DNA, 27 bp, Artificial sequence
  Single strand DNA oligonucleotide
aatataaatatatcccatcccttattt

SEQ ID NO: 502 - DNA, 22 bp, Artificial sequence
  Single strand DNA oligonucleotide
cagagtaaaaatgtaacgctga

SEQ ID NO: 503 - DNA, 26 bp, Artificial sequence
  Single strand DNA oligonucleotide
cccatcccttatttaagttatttttc

SEQ ID NO: 504 - DNA, 20 bp, Artificial sequence
  Single strand DNA oligonucleotide
gcaacatcaaaatggctgag

SEQ ID NO: 505 - DNA, 21 bp, Artificial sequence
  Single strand DNA oligonucleotide
tgattttcgtcagactgcaag

SEQ ID NO: 506 - DNA, 20 bp, Artificial sequence
  Single strand DNA oligonucleotide
gcaacatcaaaatggctgag

SEQ ID NO: 507 - DNA, 20 bp, Artificial sequence
  Single strand DNA oligonucleotide
aacctttcaaccggagttca

SEQ ID NO: 508 - DNA, 20 bp, Artificial sequence
  Single strand DNA oligonucleotide
tggccttgtaccaattcctt

SEQ ID NO: 509 - DNA, 21 bp, Artificial sequence
  Single strand DNA oligonucleotide
tgattttcgtcagactgcaag

SEQ ID NO: 510 - DNA, 20 bp, Artificial sequence
  Single strand DNA oligonucleotide
tccgatgaaacatcaccatc

SEQ ID NO: 511 - DNA, 20 bp, Artificial sequence
  Single strand DNA oligonucleotide
caattgaaaacatggccaga

SEQ ID NO: 512 - DNA, 21 bp, Artificial sequence
  Single strand DNA oligonucleotide
gatgtccgatgaaacatcacc

SEQ ID NO: 513 - DNA, 20 bp, Artificial sequence
  Single strand DNA oligonucleotide
caattgaaaacatggccaga

SEQ ID NO: 514 - DNA, 20 bp, Artificial sequence
  Single strand DNA oligonucleotide
tggccttgtaccaattcctt

SEQ ID NO: 515 - DNA, 25 bp, Artificial sequence
  Single strand DNA oligonucleotide
tttttcagtaagctcagacaaatca

SEQ ID NO: 516 - DNA, 20 bp, Artificial sequence
  Single strand DNA oligonucleotide
tggccttgtaccaattcctt

SEQ ID NO: 517 - DNA, 21 bp, Artificial sequence
  Single strand DNA oligonucleotide
aaacagatgtcccgaaaatca

SEQ ID NO: 518 - DNA, 20 bp, Artificial sequence
  Single strand DNA oligonucleotide
cctgctgatagtgccatgaa

SEQ ID NO: 519 - DNA, 20 bp, Artificial sequence
  Single strand DNA oligonucleotide
taatcaggaacagccggaag

SEQ ID NO: 520 - DNA, 20 bp, Artificial sequence
  Single strand DNA oligonucleotide
cctgctgatagtgccatgaa

SEQ ID NO: 521 - DNA, 20 bp, Artificial sequence
  Single strand DNA oligonucleotide
cggtaagacaccagccttga

SEQ ID NO: 522 - DNA, 20 bp, Artificial sequence
  Single strand DNA oligonucleotide
tccttgcctcatgtgttctg

SEQ ID NO: 523 - DNA, 20 bp, Artificial sequence
  Single strand DNA oligonucleotide
gagatcaagcgtttcccaag

SEQ ID NO: 524 - DNA, 20 bp, Artificial sequence
  Single strand DNA oligonucleotide
ggaattgcgagtgaggtctt

SEQ ID NO: 525 - DNA, 20 bp, Artificial sequence
  Single strand DNA oligonucleotide
agcgaacgaacagctcagat

SEQ ID NO: 526 - DNA, 20 bp, Artificial sequence
  Single strand DNA oligonucleotide
aggtcttaccattggctgga

SEQ ID NO: 527 - DNA, 20 bp, Artificial sequence
  Single strand DNA oligonucleotide
tgtcgagaataagcgaacga

SEQ ID NO: 528 - DNA, 21 bp, Artificial sequence
  Single strand DNA oligonucleotide
cactttgcatgaaagggctta

SEQ ID NO: 529 - DNA, 20 bp, Artificial sequence
  Single strand DNA oligonucleotide
agcgaacgaacagctcagat

SEQ ID NO: 530 - DNA, 20 bp, Artificial sequence
  Single strand DNA oligonucleotide
aggatgctggatcaaaatgc

SEQ ID NO: 531 - DNA, 20 bp, Artificial sequence
  Single strand DNA oligonucleotide
ttgatagcttagcgcccaat

SEQ ID NO: 532 - DNA, 21 bp, Artificial sequence
  Single strand DNA oligonucleotide
tctcaccttcactttgcatga

SEQ ID NO: 533 - DNA, 20 bp, Artificial sequence
  Single strand DNA oligonucleotide
agcgaacgaacagctcagat

SEQ ID NO: 534 - DNA, 22 bp, Artificial sequence
  Single strand DNA oligonucleotide
tcacatgtgttgcacctttaca

SEQ ID NO: 535 - DNA, 24 bp, Artificial sequence
  Single strand DNA oligonucleotide
gcaaccttttgctttaatgttttt

SEQ ID NO: 536 - DNA, 20 bp, Artificial sequence
  Single strand DNA oligonucleotide
tgcacaactggcctatgttt

SEQ ID NO: 537 - DNA, 24 bp, Artificial sequence
  Single strand DNA oligonucleotide
gcaaccttttgctttaatgttttt

SEQ ID NO: 538 - DNA, 20 bp, Artificial sequence
  Single strand DNA oligonucleotide
ggagagtgcggggtatttta

SEQ ID NO: 539 - DNA, 20 bp, Artificial sequence
  Single strand DNA oligonucleotide
agttcattcgcaaccgtttt

SEQ ID NO: 540 - DNA, 20 bp, Artificial sequence
  Single strand DNA oligonucleotide
tgctattggtgaatggcaaa

SEQ ID NO: 541 - DNA, 20 bp, Artificial sequence
  Single strand DNA oligonucleotide
cccgcttcatctgatggtat

SEQ ID NO: 542 - DNA, 20 bp, Artificial sequence
  Single strand DNA oligonucleotide
gttggcaggttgctcaattc

SEQ ID NO: 543 - DNA, 20 bp, Artificial sequence
  Single strand DNA oligonucleotide
ctccaccataattccgcttg

SEQ ID NO: 544 - DNA, 22 bp, Artificial sequence
  Single strand DNA oligonucleotide
tgagcaagagcataggttttga

SEQ ID NO: 545 - DNA, 20 bp, Artificial sequence
  Single strand DNA oligonucleotide
cgcaaccgttttgttttctt

SEQ ID NO: 546 - DNA, 20 bp, Artificial sequence
  Single strand DNA oligonucleotide
gttggcaggttgctcaattc

SEQ ID NO: 547 - DNA, 20 bp, Artificial sequence
  Single strand DNA oligonucleotide
tgccattcaccaatagcaaa

SEQ ID NO: 548 - DNA, 20 bp, Artificial sequence
  Single strand DNA oligonucleotide
gcggaattatggtggagaaa

SEQ ID NO: 549 - DNA, 20 bp, Artificial sequence
  Single strand DNA oligonucleotide
atagacgtcgccagatttcc

SEQ ID NO: 550 - DNA, 20 bp, Artificial sequence
  Single strand DNA oligonucleotide
tacaaaggctgttggcaggt

SEQ ID NO: 551 - DNA, 20 bp, Artificial sequence
  Single strand DNA oligonucleotide
tgccattcaccaatagcaaa

SEQ ID NO: 552 - DNA, 21 bp, Artificial sequence
  Single strand DNA oligonucleotide
ggatgatgaatcaatgccaaa

SEQ ID NO: 553 - DNA, 20 bp, Artificial sequence
  Single strand DNA oligonucleotide
cacttccgaattgagcaacc

SEQ ID NO: 554 - DNA, 27 bp, Artificial sequence
  Single strand DNA oligonucleotide
cgagcactgttatagtaatttcagaag

SEQ ID NO: 555 - DNA, 20 bp, Artificial sequence
  Single strand DNA oligonucleotide
gagtggtcggggtattgtgt

SEQ ID NO: 556 - DNA, 20 bp, Artificial sequence
  Single strand DNA oligonucleotide
tgaagtttgtgccacggtaa

SEQ ID NO: 557 - DNA, 20 bp, Artificial sequence
  Single strand DNA oligonucleotide
gatgagtggtcggggtattg

SEQ ID NO: 558 - DNA, 27 bp, Artificial sequence
  Single strand DNA oligonucleotide
cgagcactgttatagtaatttcagaag

SEQ ID NO: 559 - DNA, 20 bp, Artificial sequence
  Single strand DNA oligonucleotide
cgacctgaaaagcccaaata

SEQ ID NO: 560 - DNA, 20 bp, Artificial sequence
  Single strand DNA oligonucleotide
acagtcgagccagcttcaat

SEQ ID NO: 561 - DNA, 20 bp, Artificial sequence
  Single strand DNA oligonucleotide
cgacctgaaaagcccaaata

SEQ ID NO: 562 - DNA, 20 bp, Artificial sequence
  Single strand DNA oligonucleotide
acagtcgagccagcttcaat

SEQ ID NO: 563 - DNA, 20 bp, Artificial sequence
  Single strand DNA oligonucleotide
cccaaatacttcgggagcta

SEQ ID NO: 564 - DNA, 20 bp, Artificial sequence
  Single strand DNA oligonucleotide
ccaccatcaccctcaagttc

SEQ ID NO: 565 - DNA, 20 bp, Artificial sequence
  Single strand DNA oligonucleotide
tgacatggttgacaacagca

SEQ ID NO: 566 - DNA, 20 bp, Artificial sequence
  Single strand DNA oligonucleotide
cccgaagtatttgggctttt

SEQ ID NO: 567 - DNA, 20 bp, Artificial sequence
  Single strand DNA oligonucleotide
gagggtgatggtggtgatgt

SEQ ID NO: 568 - DNA, 21 bp, Artificial sequence
  Single strand DNA oligonucleotide
ttgaatcaaaaatgcacgaca

SEQ ID NO: 569 - DNA, 20 bp, Artificial sequence
  Single strand DNA oligonucleotide
tgaacttgagggtgatggtg

SEQ ID NO: 570 - DNA, 20 bp, Artificial sequence
  Single strand DNA oligonucleotide
cccgaagtatttgggctttt

SEQ ID NO: 571 - DNA, 20 bp, Artificial sequence
  Single strand DNA oligonucleotide
caaatagtccccgcccttta

SEQ ID NO: 572 - DNA, 20 bp, Artificial sequence
  Single strand DNA oligonucleotide
ccgaaaagaggagttgaacg

SEQ ID NO: 573 - DNA, 20 bp, Artificial sequence
  Single strand DNA oligonucleotide
tgacatggttgacaacagca

SEQ ID NO: 574 - DNA, 25 bp, Artificial sequence
  Exemplary probe that may be used to identify M17
ttgtccatcttcctgatattggtat

SEQ ID NO: 575 - DNA, 20 bp, Artificial sequence
  Exemplary probe that may be used to identify M17
tcttcccggaaaatgagatg

SEQ ID NO: 576 - DNA, 25 bp, Artificial sequence
  Exemplary probe that may be used to identify M17
tgtatcaagctttcaacgttactga

SEQ ID NO: 577 - DNA, 20 bp, Artificial sequence
  Exemplary probe that may be used to identify M17
gtgcggtgaaaaaggtcatt

SEQ ID NO: 578 - DNA, 25 bp, Artificial sequence
  Exemplary probe that may be used to identify M17
ggttactttttgttcaagtcagcat

SEQ ID NO: 579 - DNA, 20 bp, Artificial sequence
  Exemplary probe that may be used to identify M17
gagggcgataatgaaatcga

SEQ ID NO: 580 - DNA, 25 bp, Artificial sequence
  Exemplary probe that may be used to identify M17
gtaggtaaaggtctggatggtagtg

SEQ ID NO: 581 - DNA, 20 bp, Artificial sequence
  Exemplary probe that may be used to identify M17
tgtgtggaatggtgctgttt

SEQ ID NO: 582 - DNA, 25 bp, Artificial sequence
  Exemplary probe that may be used to identify M17
cagatacaccggatatttaggaatg

SEQ ID NO: 583 - DNA, 20 bp, Artificial sequence
  Exemplary probe that may be used to identify M17
cgtcgggtcaaggataggta

SEQ ID NO: 584 - DNA, 25 bp, Artificial sequence
  Exemplary probe that may be used to identify M17
ccgctagataaaaactgtattgcat

SEQ ID NO: 585 - DNA, 20 bp, Artificial sequence
  Exemplary probe that may be used to identify M17
gttgtggagcagcttgaaca

SEQ ID NO: 586 - DNA, 25 bp, Artificial sequence
  Exemplary probe that may be used to identify M17
aaagtgttttcaattcaacaggaag

SEQ ID NO: 587 - DNA, 20 bp, Artificial sequence
  Exemplary probe that may be used to identify M17
ggtgctagactctgggcttg

SEQ ID NO: 588 - DNA, 25 bp, Artificial sequence
  Exemplary probe that may be used to identify M17
gtagtcgtcaagccttcattcttta

SEQ ID NO: 589 - DNA, 20 bp, Artificial sequence
  Exemplary probe that may be used to identify M17
actaagcagaagccgccata

SEQ ID NO: 590 - DNA, 25 bp, Artificial sequence
  Exemplary probe that may be used to identify M17
gttccgttcctctggtaaattagtt

SEQ ID NO: 591 - DNA, 20 bp, Artificial sequence
  Exemplary probe that may be used to identify M17
gagctttggcttaagggctt

SEQ ID NO: 592 - DNA, 25 bp, Artificial sequence
  Exemplary probe that may be used to identify M17
gcttaagtacggtgacattgttctt

SEQ ID NO: 593 - DNA, 20 bp, Artificial sequence
  Exemplary probe that may be used to identify M17
ctacccgtggcacagtaggt

SEQ ID NO: 594 - DNA, 25 bp, Artificial sequence
  Exemplary probe that may be used to identify M17
ggagaacaaagatttttacccaatt

SEQ ID NO: 595 - DNA, 20 bp, Artificial sequence
  Exemplary probe that may be used to identify M17
ggaaacaaaccgactggaaa

SEQ ID NO: 596 - DNA, 25 bp, Artificial sequence
  Exemplary probe that may be used to identify M17
aatatattactggggctaaagtccg

SEQ ID NO: 597 - DNA, 20 bp, Artificial sequence
  Exemplary probe that may be used to identify M17
tttccagtggcgatctagct

SEQ ID NO: 598 - DNA, 25 bp, Artificial sequence
  Exemplary probe that may be used to identify M17
atctaatcatgtaccgacatcaggt

SEQ ID NO: 599 - DNA, 20 bp, Artificial sequence
  Exemplary probe that may be used to identify M17
ggcaagcagattgtatcggt

SEQ ID NO: 600 - DNA, 25 bp, Artificial sequence
  Exemplary probe that may be used to identify M17
cttggtattgggaaaaagatatcct

SEQ ID NO: 601 - DNA, 20 bp, Artificial sequence
  Exemplary probe that may be used to identify M17
gaaattatgggagcaaggca

SEQ ID NO: 602 - DNA, 25 bp, Artificial sequence
  Exemplary probe that may be used to identify M17
gacggataaacagatccacaattac

SEQ ID NO: 603 - DNA, 20 bp, Artificial sequence
  Exemplary probe that may be used to identify M17
gggcagactatcaggcagag

SEQ ID NO: 604 - DNA, 25 bp, Artificial sequence
  Exemplary probe that may be used to identify M17
gtcagacaggcaaatccatagatag

SEQ ID NO: 605 - DNA, 20 bp, Artificial sequence
  Exemplary probe that may be used to identify M17
gaggcataaacccatgctgt

SEQ ID NO: 606 - DNA, 25 bp, Artificial sequence
  Exemplary probe that may be used to identify M17
ggacattaatatctgtgggtgagtc

SEQ ID NO: 607 - DNA, 20 bp, Artificial sequence
  Exemplary probe that may be used to identify M17
ttgaatttattcgcccgaac

SEQ ID NO: 608 - DNA, 25 bp, Artificial sequence
  Exemplary probe that may be used to identify M17
gagaatgtgacgtttatgtgttcag

SEQ ID NO: 609 - DNA, 20 bp, Artificial sequence
  Exemplary probe that may be used to identify M17
ccagtcagtgagctatggca

SEQ ID NO: 610 - DNA, 25 bp, Artificial sequence
  Exemplary probe that may be used to identify M17
agagcgttaagttttggtatcaatg

SEQ ID NO: 611 - DNA, 20 bp, Artificial sequence
  Exemplary probe that may be used to identify M17
acagcaactgcgtctttcct

SEQ ID NO: 612 - DNA, 25 bp, Artificial sequence
  Exemplary probe that may be used to identify M17
tctttcactgcataaattaaatgca

SEQ ID NO: 613 - DNA, 20 bp, Artificial sequence
  Exemplary probe that may be used to identify M17
tcaacctaatgcaaatgcca

SEQ ID NO: 614 - DNA, 25 bp, Artificial sequence
  Exemplary probe that may be used to identify M17
ttctcttgagcgaagtgttttagtt

SEQ ID NO: 615 - DNA, 20 bp, Artificial sequence
  Exemplary probe that may be used to identify M17
acgccagagaatctggctaa

SEQ ID NO: 616 - DNA, 25 bp, Artificial sequence
  Exemplary probe that may be used to identify M17
ttgttatcactgaatacttggggtt

SEQ ID NO: 617 - DNA, 20 bp, Artificial sequence
  Exemplary probe that may be used to identify M17
cccgtttgggtgataatgtc

SEQ ID NO: 618 - DNA, 25 bp, Artificial sequence
  Exemplary probe that may be used to identify M17
tatcactgttaggttgggaatgaat

SEQ ID NO: 619 - DNA, 20 bp, Artificial sequence
  Exemplary probe that may be used to identify M17
gaaaaggttgcttgacgctc

SEQ ID NO: 620 - DNA, 25 bp, Artificial sequence
  Exemplary probe that may be used to identify M17
gagataatgagtcctcttctttccc

SEQ ID NO: 621 - DNA, 20 bp, Artificial sequence
  Exemplary probe that may be used to identify M17
gggttggatcattgttccac

SEQ ID NO: 622 - DNA, 25 bp, Artificial sequence
  Exemplary probe that may be used to identify M17
cattaggacttttgtgcaccttagt

SEQ ID NO: 623 - DNA, 20 bp, Artificial sequence
  Exemplary probe that may be used to identify M17
gtcgctttgctgcatattga

SEQ ID NO: 624 - DNA, 25 bp, Artificial sequence
  Exemplary probe that may be used to identify M17
cagtaatcgttttactgtccgaact

SEQ ID NO: 625 - DNA, 20 bp, Artificial sequence
  Exemplary probe that may be used to identify M17
tctcgatgtactgctggtgc

SEQ ID NO: 626 - DNA, 25 bp, Artificial sequence
  Exemplary probe that may be used to identify M17
cagcttcgacttgtatcagtagaca

SEQ ID NO: 627 - DNA, 20 bp, Artificial sequence
  Exemplary probe that may be used to identify M17
atacgttttcacgccgtttc

SEQ ID NO: 628 - DNA, 25 bp, Artificial sequence
  Exemplary probe that may be used to identify M17
aacgacgtaaagaactcaaaatgac

SEQ ID NO: 629 - DNA, 20 bp, Artificial sequence
  Exemplary probe that may be used to identify M17
cgaccctaattggctgttgt

SEQ ID NO: 630 - DNA, 25 bp, Artificial sequence
  Exemplary probe that may be used to identify M17
agttatcgactatcaacggtgaaag

SEQ ID NO: 631 - DNA, 20 bp, Artificial sequence
  Exemplary probe that may be used to identify M17
gcggtggctacactatggtt

SEQ ID NO: 632 - DNA, 25 bp, Artificial sequence
  Exemplary probe that may be used to identify M17
cacctgaacttcttgagagagtttc

SEQ ID NO: 633 - DNA, 20 bp, Artificial sequence
  Exemplary probe that may be used to identify M17
atcgcggtaacacttggttc

SEQ ID NO: 634 - DNA, 25 bp, Artificial sequence
  Exemplary probe that may be used to identify M17
agcaagtctctcaaaacctacagaa

SEQ ID NO: 635 - DNA, 20 bp, Artificial sequence
  Exemplary probe that may be used to identify M17
tttacctatggctgttgccc

SEQ ID NO: 636 - DNA, 25 bp, Artificial sequence
  Exemplary probe that may be used to identify M17
tttttgttaaatgatgcgcattata

SEQ ID NO: 637 - DNA, 20 bp, Artificial sequence
  Exemplary probe that may be used to identify M17
tttttgttaaatgatgcgca

SEQ ID NO: 638 - DNA, 25 bp, Artificial sequence
  Exemplary probe that may be used to identify M17
aaaaatagatgataacggaaaaggg

SEQ ID NO: 639 - DNA, 20 bp, Artificial sequence
  Exemplary probe that may be used to identify M17
tggtgatatttcgtccccat

SEQ ID NO: 640 - DNA, 25 bp, Artificial sequence
  Exemplary probe that may be used to identify M17
gaaaatggtaagaaagaagcattga

SEQ ID NO: 641 - DNA, 20 bp, Artificial sequence
  Exemplary probe that may be used to identify M17
cgctgtggaaagtgacagaa

SEQ ID NO: 642 - DNA, 25 bp, Artificial sequence
  Exemplary probe that may be used to identify M17
taccgctgtattaaattagtgtgca

SEQ ID NO: 643 - DNA, 20 bp, Artificial sequence
  Exemplary probe that may be used to identify M17
tgcgaatgaactcacaggag

SEQ ID NO: 644 - DNA, 25 bp, Artificial sequence
  Exemplary probe that may be used to identify M17
ggatacgagcaaataatacatcacc

SEQ ID NO: 645 - DNA, 20 bp, Artificial sequence
  Exemplary probe that may be used to identify M17
acagtcgagccagcttcaat

SEQ ID NO: 646 - DNA, 393 bp, Escherichia coli strain M17
ctttacatttcactatgtgattattttttaattgtatacacattcttggattttgaagttgagattatccttcataaagaaggatgaagagttcccttttaaaaatgactctatgctatttatgtgcatatcataatcatcaatgagcaccttatcttgggatgaaccaataaaaataacattgaccttatctgcactaaggttaatgcaatgatgatttatattaaagtctaacaagtgagcaagagcataggttttgactggaaggttttcttcactgaaataactattttgccaagataaaatgaatataaaaattaaaaaagaaaacaaaacggttgcgaatgaactcacaggagggagatcgaaagcttcatctttaaataaaaaaca

SEQ ID NO: 647 - DNA, 958 bp, Escherichia coli strain M17
gtgtataaggaaaggatgatgaatcaatgccaaatacactatttaaaagtgctgatgattttgcggtgcaaaaaataatcaatcctgatatggctatagaggtaatacattttgtaattattgagttccatagcaccttaaaaatcaatgttctttcaataaatacacctaaccataatgtagaaaaaattattgataatagtatgcatgtaaattgccattgagagaaccaatcatctgagtctgtctttgctatacctaaagccaaacatatgagagctaaaatgcctatcacaattaaagcatatgatttctctgattcatcctgacgttttttaagctcatcatagttttgtaacttttttacaaaggctgttggcaggttgctcaattcggaagtgtcattcttcatatgttctccaagataacaacgatatgctggaagtataccgctgtattaaattagtgtgcaagcggaattatggtggagaaacattcataatatcaattttattgtatgtgtctatttgacgtatctctgtattgatgataactaaatataacattcaataaccaaaggagataaaaaacgaatgcacaactggcctatgtttttactattgatactcgtattcatcagaacagtaatgaaaagattactgaaattttcattgcgaaatataaagaaggaataacgtatcattttggccctgatatggaacgatttgagtcctgctttgaagcagcgctttttgctattggtgaatggcaaataaaagttgatgcgatgacacgataccatgaggaagatgaagtgatggagttctttagctctataccatcagatgaagcgggaaatctggcgacgtctattttgctgttcagtgatgttctcgccatgaaaggcgtggatgaagtatttgcctattttttgggtaaggatgatgttgtatgagtg